(19) United States
(12) Patent Application Publication
(10) Pub. No.: US 2012/0185964 A1
(43) Pub. Date: Jul. 19, 2012

(54) METHODS AND COMPOSITIONS FOR STRESS TOLERANCE IN PLANTS

(76) Inventors: Julieta Virginia Cabello, Santa Fe (AR); Agustin Lucas Arce, Santa Fe (AR); Raquel Lia Chan, Santa Fe (AR)

(21) Appl. No.: 13/375,430

(22) PCT Filed: Jun. 1, 2010
(86) PCT No.: PCT/GB2010/050920

§ 371 Date: Mar. 27, 2012

(30) Foreign Application Priority Data
Jun. 1, 2009 (GB) 0909318.8

(51) Int. Cl. A01H 5/00; C12N 5/10; C07K 14/415; C40B 30/04; C12N 15/82; C12N 15/29

(52) U.S. Cl. 800/279; 800/298; 800/301; 800/278; 800/289; 536/23.6; 530/370; 506/9; 435/320.1; 435/419

(57) ABSTRACT

We characterise a plant transcription factor and disclose its use in modifying plant responses to stress conditions, including freezing, drought, salinity and pathogen invasion. Microarray analyses were performed indicating that such tolerance occurs via the increase of antifreeze proteins localized in the cellular apoplast which inhibit the growth of large extracellular ice crystals. We also disclose the use of such proteins.

### FIELD OF THE INVENTION

The invention relates to transgenic plants with enhanced tolerance to biotic and abiotic stress and to methods for making such plants. Also within the scope of the invention are isolated nucleic acid and peptide sequences.

### BACKGROUND OF THE INVENTION

Adverse climate conditions and human activity as well as biological agents are stress effectors for plants and seriously affect their productivity and survival. Losses in productivity due to this kind of stress reach sometimes more than 50%. Plant breeders have been and are devoted to developing strategies in order to avoid or diminish the negative impact of these situations.

Among abiotic stress-causing factors, drought, salinity of the soil and extreme temperatures are some of the most harmful. Regarding extreme temperatures, stresses are classified into three types: freezing (caused by temperatures below 0° C.), chilling (caused by low temperatures over 0° C.) and heat stress (caused by high temperatures). Chilling temperatures cause damage to photosynthetic tissues, inhibit the whole photosynthetic process and carbohydrate transport as well as protein biosynthesis and respiration rates. Simultaneously, protein degradation is accelerated. All of these effects occur rather slowly and involve partial or total loss of membrane functionality. In contrast, freezing temperatures cause quick damage, killing the plants. It has been observed, however, that plants subjected to chilling during several days tolerate freezing temperatures better than plants subjected to freezing without first having been exposed to a period of chilling; this process is termed “acclimation”.

Species such as winter cereals are adapted to cold or moderate-cold weather and can tolerate temperatures ranging from 0° C. to 15° C., as well as freezing temperatures, rather well if they have previously been acclimated to reduced temperatures (Levitt, 1980, Thomashow, 1999). By contrast, tropical and subtropical species, including important crops, such as maize, rice or tomato, are sensitive to low temperatures and appear to lack efficient acclimation mechanisms.

Chilling and freezing tolerance occurs via different mechanisms. The response to chilling involves the activation of unsaturases which are able to change the lipid composition of the membranes generating increased membrane fluidity at low temperatures. On the other hand, freezing tolerance requires a previous acclimation period. During this acclimation period, certain specific proteins are synthesized and accumulated.

“Antifreeze” proteins are found in a wide range of overwintering plants; they inhibit the growth and re-crystallization of ice produced in intercellular spaces at freezing temperatures. These proteins exhibit a high level of homology with pathogenesis-related proteins (PRs) and, in some cases, also protect against psychrophilic pathogens (Griffith & Yaish, 2004; Chinnusamy et al., 2007). Other species present freezing tolerance via a mechanism implying the increase in sucrose (Guy, 1992) or free proline concentrations (Nanjo et al., 1999).

One of the strategies to reduce losses in plant productivity is to increase natural stress tolerance, by strengthening endogenous systems. Transcription factors (TFs) play a crucial role in the plant response to environmental factors as well as in the morphogenetic program. They are proteins acting in trans, able to recognize and bind specific DNA sequences (cis-acting elements) localized in the regulatory regions of their target genes. When these proteins bind their targets, they activate or repress whole transduction signals pathways.

About 1500 TFs have been identified in plants using bioinformatics, and TFs comprise numerous gene families. However, while they might be involved in the response, they may not necessarily confer a tolerance. This is illustrated, for example, by Arabidopsis TFs ATHB7 and ATHB12 (Lee and Chun, 1998). These exhibit a high homology with sunflower HAHB4, especially in the HD-Zip domain. Both genes are up-regulated by drought and ABA. However, it was also shown that transgenic plants overexpressing these genes are not more tolerant to drought stress than WT ones. HAHB4 is described in WO 2004/099365. Another example is DREB2, a gene that is induced by cold temperatures, but does not confer cold tolerance in its wild type form. Therefore, it continues to be necessary to undertake a series of functional genomic experiments in order to test and demonstrate the effects of TFs on stress tolerance as such effects cannot be predicted (Arce et al., 2008).

HD-Zip proteins characterized by the presence of a homeodomain associated with a leucine zipper constitute one family of plant transcription factors. The association of the DNA binding domain (HD) with an adjacent dimerization motif (leucine zipper abbreviated ZipLZ or LZ) is a combination found only in the plant kingdom, although the domains are found independently of each other in a large number of eukaryotic transcription factors (Schena & Davis, 1992). This large family of plant TFs has been divided into four subfamilies (I to IV) according to sequence similarity in and outside the conserved domains and by the intron/exon patterns of the corresponding genes (Schena & Davis, 1994, Sessa et al., 1994, Chan et al., 1998; Ariel et al., 2007). Members of subfamily I interact with the pseudopalindromic sequence CAAT(A/T)ATTG; subfamily II proteins recognize a motif CAAT(C/G)ATTG (Sessa et al., 1993; Palena et al., 1999). In all cases, the formation of protein homo- or hetero-dimers is a prerequisite for DNA binding (Sessa et al., 1993; Gonzalez et al., 1997).

Several authors have reported that expression of members of the HD-Zip family of transcription factors is regulated by various external factors such as illumination, ABA, salt or water stresses (Schena & Davis, 1992; Carabelli et al., 1993; Schena et al., 1993; Soderman et al., 1994; Soderman et al., 1996, Chan et al., 1998; Lee & Chun, 1998; Soderman et al., 1999a and 1999b; Gago et al., 2002; Henriksson et al., 2005). Studies in which HD-Zip I and II genes were overexpressed in transgenic plants further support the proposed role of this protein family as developmental regulators that are responsive to environmental conditions (Schena et al., 1993; Manavella et al., 2006; Manavella et al., 2008; Ariel et al., 2007; Cabello et al., 2007, Dezar et al., 2005a). There remains the need to identify and characterize such proteins in a way that beneficial traits can be conferred on plants utilizing specific members of this class of molecules.

HAHB1 cDNA was isolated in 1992 from a sunflower stem cDNA library and its sequence was deposited in the Genebank (accession number L22847, see SEQ. ID. NO:2: herein for the nucleic acid sequence and SEQ ID. NO:5: herein for the translated protein sequence) and the cloning of the cDNA was described (Chan R L, Gonzalez D H, 1994). The protein encoded by this gene has been referenced in the literature as a gene homologous to HD-Zip proteins from other species, but this conclusion is based only on comparison of the sequence in phylogenetic trees (Gonzalez et al 1997, Chan et al, 1998 and Ariel et al, 2007).

The present invention surprisingly demonstrates the utility of HAHB1 (Helianthus Annuus Homeobox 1), in the production of transgenic plants with enhanced tolerance to stress conditions.

### SUMMARY OF THE INVENTION

In the present patent disclosure, we describe the use of HAHB1, a transcription factor that is a member of the sunflower subfamily I of HD-Zip proteins and variants thereof, such as ATHB13, to modify plant responses to stress conditions, including freezing, drought, salinity and biotic stress. Thus, the various aspects of the invention all relate to plants and method which confer or increase such stress tolerance. The gene was isolated from a genomic library and its expression pattern characterized. We demonstrate that by making transgenic plants bearing the sunflower HAHB1 cDNA under the control of either the constitutive 35S promoter or the native HAHB1 promoter, transgenic plants are produced which exhibit clear increases in tolerance to low temperature conditions in the vegetative and reproductive stages. In addition, the transgenic plants exhibit better tolerance than non-transformed plants in response to drought or salinity conditions. Similar effects are observed when using the HAHB1 homologue ATHB13. Microarray analyses were performed to assess expression patterns in transgenic Arabidopsis plants. The data indicates that the observed tolerance occurs via the increase of antifreeze proteins localized in the cellular apoplast which inhibit the growth of large extracellular ice crystals. Moreover, the inventors have also shown that the transient overexpression of the HAHB1 gene in sunflower induces the expression of several genes related to stress tolerance.

HAHB1 is a member of the HD-Zip subfamily I (HD-Zip I). All HD-Zip I family members show high sequence similarity in the N-terminal homeodomain (HD) and leucine zipper domain (LZ), but are notably much more diverse in the C-terminal region. For example, HAHB1 and HAHB4 are both members of HD-Zip I, but they share very little sequence similarity in their C-terminal regions. Although HD-Zip I are grouped into a single family, different HD-Zip I family members exhibit differential expression patterns and are involved in different developmental and physiological processes as detailed below. The inventors have characterised HAHB1 and compared its structure to homologous sequences. Using chimeric constructs, the inventors have also found that it is the C-terminus of the HAHB1 protein that is important in conferring HAHB1 function. Genes homologous to HAHB1 that show high homology not only in the HD and LZ domains, but also in the C-terminal domains, are predicted to have a similar effect as HAHB1 when expressed in transgenic plants, as shown herein for ATHB13.

From the information provided herein, those skilled in the art will appreciate that HAHB1, a part or a homolog thereof, confers enhanced tolerance, for example, freezing tolerance to transgenic plants if it is expressed, either under the control of its own promoter or under the control of a constitutive promoter such as the CaMV 35S promoter, or under the control of another type of promoter, such as for example a cold inducible promoter or an abiotic stress inducible promoter.

This freezing tolerance is accompanied by enhanced tolerance to drought and high salt conditions. We also show that not only can HAHB1 be expressed under the control of the HAHB1 promoter to achieve stress-responsive expression, but any gene sequence may be operatively associated with the HAHB1 promoter in order to achieve expression under cold or freezing conditions, high salt or low water or pathogen invasion.

As stated above, without wishing to be bound by any particular theory, the tolerance to freezing conditions conferred by expression of HAHB1 is apparently due to the synthesis and action of antifreeze proteins in the cellular apoplast inhibiting ice crystal formation with its subsequent dehydration effects. It also is possible that other proteins outside the apoplast (inside the cell) are also up translated.

In light of the general information provided herein, those skilled in the art will appreciate that this invention describes and enables those skilled in the art to obtain and isolate a gene sequence from sunflower which can be used to confer enhanced tolerance to stress conditions including enhanced chilling and freezing tolerance, enhanced tolerance to drought, enhanced tolerance to conditions of high salinity and/or biotic stress. In addition, using methods known in the art, the isolated gene sequence from sunflower disclosed herein or parts thereof or can be used to isolate related sequences from other plants which can be used to confer enhanced tolerance to abiotic and biotic stress conditions as described herein.

Accordingly, it is an object of this invention to provide an isolated protein or gene sequence from sunflower, HAHB1, a part or variant thereof, which can be used to confer enhanced tolerance to abiotic and biotic stress conditions as described herein, even in plant species other than sunflower.

It is a further object of this invention to provide an isolated gene sequence from sunflower, HAHB1, a part or variant thereof, which can be used to isolate related sequences from other plants, including plant species not related to sunflower, which sequences can be used to confer enhanced tolerance to abiotic and biotic stress conditions as described herein.

It is a further object of this invention to provide an isolated promoter gene sequence which can be used to regulate expression of sequences operatively associated with the promoter sequence to confer on the thus associated sequences the property of expression in response to abiotic and biotic stress conditions as described herein.

It is a further object of this invention to provide expression constructs or vectors comprising nucleic acid sequences described herein which confer on plants into which such expression constructs or vectors are introduced enhanced tolerance to abiotic and biotic stress conditions as described herein.

It is a further object of this invention to provide transgenic plants which have enhanced or increased tolerance to abiotic and biotic stress conditions as described herein.

It is a further object of this invention to provide compositions and methods to induce production of antifreeze proteins (AFPs) in a plant to thereby avoid or minimize damage otherwise caused in plants on exposure to low temperature conditions as a result of ice crystal formation.

It is a further object of this invention to provide novel methods for identifying and using novel compositions disclosed herein to confer enhanced tolerance to abiotic and biotic stress conditions as described herein.

Thus, based on the information provided herein, those skilled in the art will be able to prepare expression constructs or vectors comprising nucleic acid sequences which confer on plants into which such expression constructs or vectors are introduced enhanced tolerance to abiotic and biotic stress conditions as described herein. This will enable those skilled in the art to produce plants which have enhanced tolerance to abiotic and biotic stress conditions as described herein.

Without wishing to be bound by theory, those skilled in the art will appreciate that the compositions and methods provided herein will permit induction of production of antifreeze proteins (AFPs) in a plant to thereby avoid or minimize damage otherwise caused in plants on exposure to low temperature conditions as a result of ice crystal formation.

Other objects, advantages and benefits of this invention will be apparent to those skilled in the art from a review of the complete disclosure provided herein and the appended claims.

### BRIEF DESCRIPTION OF THE DRAWINGS

FIG. 1. HAHB1 expression pattern in seedlings and mature plants

a: Transcript levels of HAHB1 in different tissues and organs of sunflower 7-day-old seedlings (from left to right: root, hypocotyl, cotelydon, apical meristem); b: Transcript levels of HAHB1 quantified in different tissues and organs of sunflower 21-day-old plants (from left to right: root, hypocotyl, cotelydon, stem, leaf, petiole); transcript levels were determined by quantitative RT-PCR and standard deviations calculated from three independent samples in which actin transcripts (ACTIN2 plus ACTING) were used as internal controls; all the values are normalized with respect to the value measured in roots, arbitrarily assigned a value of one. The y axis indicates the (x fold) change observed.

FIG. 2. Sunflower HAHB1 is up-regulated by cytokines in seedlings and by ABA in mature plants

a: Transcript levels of HAHB1 in 7-day-old seedlings two hours after treatments with different hormones (from left to right: control, ABA, BAP, GA, IAA, JA, SA); b: Transcript levels of HAHB1 in 21-day-old leaves disks two hours after treatments with different hormones (from left to right: control, IAA, ACC, SA, JA, ABA, GA, BAP); transcript levels were determined by quantitative RT-PCR and standard deviations calculated from three independent samples in which actin transcripts (ACTIN2 plus ACTIN8) were used as internal controls; all the values are normalized with respect to the value measured in roots, arbitrarily assigned a value of one. The y axis indicates the (x fold) change observed.

FIG. 3. Sunflower HAHB1 is up-regulated by abiotic stress factors

a: Transcript levels of HAHB1 in 7-day-old seedlings two hours after treatment with abiotic stress factors as stated in the figure and detailed in the Experimental Procedures section (from left to right: control, drought, NaCl, dark, sucrose, H₂O₂) b: Kinetics of induction of HAHB1 in 7-day-old seedlings placed at 4° C. (from left to right: control, 3, 4, 5, 6, 7, 8hs); c: Transcript levels of HAHB1 in 21-day-old leaves disks two hours after treatments with abiotic stress factors as stated in the figure (from left to right: control, 4° C., UV-B, NaCl, dark, drought); transcript levels were determined by quantitative RT-PCR and standard deviations calculated from three independent samples in which actin transcripts (ACTIN2 plus ACTIN8) were used as internal controls; all the values are normalized with respect to the value measured in roots, arbitrarily assigned a value of one. The y axis indicates the (x fold) change observed.

FIG. 4. Morphological and developmental characteristics of transgenic plants bearing the construct 35S:HAHB1

a: 21-old-day leaves of transgenic and WT plants. Transgenic plants exhibit serrated leaves while WT plants do not. The arrows designate the serrated borders of the transgenic plants. b: Stem height during the life cycle of transgenic and WT plants; c: 30-day-old plants cultured in normal growth conditions. FIG. 4a shows the difference in the leaf morphology between transgenic and wild type plants. Transformed plants show serrated borders and a differentiated shape in comparison with their non-transformed counterparts (FIGS. 4a and 4c). In 4c, transgenic (35S:HAHB1; three independent lines A, B and C) and WT plants show similar developmental characteristics. The plants are healthy in normal growth conditions.

FIG. 5. Transgenic plants ectopically and constitutively expressing 35S:HAHB1 are more tolerant to freezing conditions

a: Membrane stability of transgenic and non-transformed plants measured as the conductivity of the supernatant solution (see Experimental Procedures, inverse to stability) after freezing treatments; b: Photograph taken 6 days after placing the plants to recover in normal growth conditions after a treatment of 8 hours in freezing conditions: WT plants are dead after a freezing treatment while transgenic plants (35S:HAHB1; three independent lines TG (transgenic) A, TG-B and TG-C) show a lesser extent of damage. Approximately, 25% of the leaves (TG genotypes) are senescent while the others are green and healthy. The x axis shows the period of time at −8° C. and the y axis shows the leaching ratio (L). Percentage of survivors of all genotypes (transgenic and non-transformed) subjected to freezing conditions as in b:

| Genotype | Survivors % |
|---|---|
| Wt | 22+-3 |
| TG-A | 85+-2 |
| TG-B | 74+-4 |
| TG-C | 70+-3 |
| TG-D | 65+-2 |
| TG-E | 60+-5 |

FIG. 6. Chlorophyll content in plants subjected to chilling conditions

Chlorophyll content was measured as described in Experimental Procedures in transgenic plants expressing HAHB1 and wild type plants subjected to chilling temperatures for the periods indicated. The values on the y axis are expressed as μg of chlorophyll per g of fresh leaves. The x axis shows the days at 4° C.

FIG. 7. Transgenic plants expressing HAHB1 are more tolerant to salt stress

a: Membrane stability of transgenic (TG-A and TG-B) plants expressing HAHB1 and non transformed plants (wt) measured as the conductivity of the supernatant solution (see Experimental Procedures, inverse to stability) after salt stress treatments; concentrations of NaCl are expressed in mM on the x axis; b: Illustrative photograph taken 2 days after watering with 200 mM NaCl. WT plants show approximately 50% of dead leaves (lighter coloured leaves) while transgenic plants exhibit more than 80% and 100% healthy green leaves for line A and B respectively. Some (but not all) dead leaves are marked with an arrow.

FIG. 8. Transgenic plants expressing HAHB1 are more tolerant to drought

Illustrative photograph taken 2 days after watering of transgenic and wild type plants subjected to drought as described in the Experimental Procedures section. Approximately 75% of the WT plants are dead as can be appreciated by the senescent leaves (lighter colour); while 25% are damaged, but present few green leaves. Transgenic plants present a different number of dead leaves (20% for line A, 50% for lines B and C) and younger leaves are green and healthy.

FIG. 9. Expression pattern of the GUS reporter gene directed by the HAHB1 promoter region.

Histochemical detection of GUS enzymatic activity in Arabidopsis transgenic plants transformed with prHAHB1:GUS. a, b, c and d: 14-day-old seedlings; e, 30 day-old plants and f, 45 day-old plant; view of meristems (a and b); cotyledons (c), hypocotyls (d), apical meristem (e) and siliques (f). GUS activity due to the expression of the gene directed by HAHB1 promoter (darker areas) is clearly visualized in the vascular system of hypocotyls, cotyledons and leaves (A-D) as well as in the meristematic region (E) and in the siliques base (F).

FIG. 10. Expression pattern of the GUS reporter gene directed by the HAHB1 promoter region in plants subjected to chilling conditions.

Histochemical detection of GUS enzymatic activity in Arabidopsis transgenic plants transformed with prHAHB1:GUS after chilling conditions; a and b, apical meristems; c and d, roots; the plants used are 30-days-old. GUS activity due to the expression of the gene directed by HAHB1 promoter (darker areas) is clearly visualized in the meristematic region (A and B) and the vascular system of roots (C and D).

FIG. 11. Morphological and developmental characteristics of transgenic plants bearing the construct promHAHB1:HAHB1

a: 25-day-old plants (wt and transgenic plants expressing HAHB1) cultured in normal growth conditions. Plants (WT and three independent transgenic genotypes) exhibit indistinguishable phenotypes in normal growth conditions. b: Stem height (y axis) during the life cycle of transgenic and WT plants.

FIG. 12. Transgenic plants expressing HAHB1 under the control of its own promoter are more tolerant to freezing conditions

a: Photograph taken 6 days after placing plants (wt and transgenic plants expressing HAHB1, previously grown during 8 hours at −8° C.) to recover in normal growth conditions; TG-A, TG-B and TG-C represent plants from three independent transgenic genotypes (construct promHAHB1:HAHB1). WT plants are senescent and almost dead (some, but not all, dead leaves are marked with an arrow) while transgenic plants exhibit certain damage in old leaves, but a healthy aspect in younger ones. b: HAHB1 transcript levels in transgenic plants bearing the construct promHAHB1:HAHB1 as a function of the incubation time at 4° C. with the y axis representing the x fold change; transcript levels were determined by quantitative RT-PCR and standard deviations calculated from three independent samples in which actin transcripts (ACTIN2 plus ACTIN8) were used as internal controls; all the values are normalized with the value measured in untreated plants (time 0), arbitrarily assigned a value of one. Percentage of survivors from the four genotypes after the freezing/recovery treatment:

| Genotype | Survivors % |
|---|---|
| Wt | 38+-5 |
| TG-A | 90+-8 |
| TG-B | 88+-6 |
| TG-C | 75+-10 |

FIG. 13. Apoplastic proteins of transgenic (35S:HAHB1) and wild type plants

SDS-PAGE showing the proteins present in the cellular apoplast, obtained from non-acclimated plants (A), plants acclimated during 16 hours at 4° C. (B), plants acclimated during 10 days at 4° C. (C) and plants placed during 3 hours at −8° C. (D, freezing treatment). All the samples were isolated from 25-day-old WT or transgenic plants (three independent lines of 35S:HAHB1 (A, B and C) or a mix of them (D). Samples loaded in A, B and C were obtained from 7 g leaf tissue and an equal volume extract was loaded in the gel. Samples loaded in D were obtained from 3 g leaf tissue and equal volumes loaded in the gel. The arrow indicates the band differentially expressed.

FIG. 14. Chromatogram of apoplastic proteins isolated from acclimated transgenic and wild type plants

Sephadex G-200 column elution chromatogram (see Experimental Procedures) of apoplastic proteins purified from both genotypes (transgenic, two independent lines expressing HAHB1: TG-A and TG-B, or wild type); the second peak in the transgenic apoplastic proteins increased to 0.8-0.9 OD compared with the WT extract in which this peak reaches 0.5-0.6 OD.

FIG. 15. Expression levels of antifreeze proteins in transgenic and wild type plants subjected to different stress treatments.

PR2 (15c and f), PR3 (15b and e) and PR4 (15a and d) expression levels in plants (control and two independent transgenic lines expressing HAHB1: TG-A and TG-B) subjected to drought (EH), 4° C. or treated with ACC (ethylene precursor), salicylic acid (SA) or abscisic acid (ABA).

FIG. 16. HAHB1 prevents the formation of large ice crystals

Apoplastic proteins were extracted and mixed with 26% sucrose and the mixture was frozen at −80° C. over a period of a few minutes; after that, the samples were gradually warmed to 0° C. and then placed for one hour at −8° C.; finally, the samples were observed and photographed with a optical microscope (×4); A, B, C, crystals formed in the presence of apoplastic transgenic proteins (three independent lines from the genotype 35S:HAHB1); D, crystals formed in the presence of apoplastic WT proteins; E, crystals formed in the presence of 26% sucrose without proteins.

FIG. 17. Transgenic plants bearing the construct 35S:GLUC are more tolerant to freezing conditions.

a: morphological characteristics of transgenic plants expressing 35S:GLUC and WT plants in normal growth conditions; non-significant differences were detected in the morphological characteristics of five independent F2 lines of transgenic plants (bearing the GLUC gene, AT4g16260) compared with control plants; b: stem height of transgenic and WT plants in later developmental stages; c: plants subjected to freezing conditions (7 hrs at −8° C.) and left in normal conditions (22-24° C.) over six days for recovery before taking the photograph; three independent lines were used in the experiment, four plants per pot of 100 g soil (7×8 cm). In 17a, WT and three independent transgenic genotypes exhibit indistinguishable phenotypes in normal growth conditions while when they were subjected to stress, all WT plants shown are wilted (17c, dead leaves are of a lighter colour) and the transgenic plants show much improved survival rates. Percentage of survivors of each genotype after the freezing treatment:

| Genotype | Survivors % |
|---|---|
| 101.3 | 8 |
| 35S: GLUC | 50 |

FIG. 18. Transgenic plants bearing the construct 35S:PR2 exhibit differential behaviour in freezing conditions as compared to their WT counterparts

a: morphological characteristics of transgenic and WT plants in normal growth conditions; non-significant differences were detected in the morphological characteristics of five independent F2 lines of transgenic plants (bearing the PR2/glucanase gene/BGL2, AT3g57260) compared with control plants; b: stem height of transgenic and WT plants in later developmental stages; c: plants subjected to freezing conditions (7 hours at −8° C.) and in normal conditions (22-24° C.) over six days of recovery before taking the photograph; three independent lines were used in the experiment, four plants per pot of 100 g soil (7×8 cm). In 18a, WT and three independent transgenic genotypes exhibit undistinguishable phenotypes in normal growth conditions while when they were subjected to stress WT plants have wilted (18c) and transgenic plants are also rather damaged as shown in c, but a few ones are still green and healthy. Percentage of survivors of each genotype after the freezing treatment:

| Genotype | Survivors % |
|---|---|
| 101.3 | 8 |
| 35S: PR2 | 17 |

FIG. 19. Sunflower apoplastic proteins are differentially expressed during cold acclimation

SDS-PAGE showing the expression pattern of apoplastic proteins obtained from plants acclimated during at 4° C. in 2 week-old sunflower plants; NA: non-acclimated plants; A12, A25 and A42 represent samples taken 12, 25 and 42 days after placing the plants at 4° C.

FIG. 20. Sunflower leaf discs transformed with 35S:HAHB1 over-express genes putatively related to the cold response

Sunflower leaves were transformed with an empty vector (121) or 35S:HAHB1 (HAHB1); transcript levels of different response genes were measured by qRT-PCR; ACTIN genes (ACTIN2 plus ACTIN8) were used as internal controls and standard deviations were calculated from at least three independent experiments; the measured genes are chitinase (A), SAG21 (B), ZAT10 (D) and DREBs (C) the function of each is described in the Results section.

FIG. 21. Comparison of members of the HD-Zip class of proteins

The sequence of HAHB1 carboxy-terminus was compared with the carboxy-termini of the most homologous proteins from other plant species found using the blast algorithm. a) to d) show the C-terminus sequence from the N- to the C-terminal end of the C-terminus. a) shows the CI motif, d) the CII motif. The sequences were aligned using the clustal algorithm (Waterhouse et al., 2009). For proteins which are not anticipated to have similar functions to HAHb1 or ATHB13, notwithstanding the high homology in the HD-Zip regions, there is significant divergence in the C-terminus. However, this comparison of each HD-Zip member also shows some degree of conservation in the carboxy-terminal motifs CI and CII. Most of the homologous proteins are not functionally characterized, but the high homology in the HD-Zip domain allows to classify them as HD-Zip proteins or genes encoding HD-Zip proteins. On the basis of the alignment, it was possible to deduce a consensus sequence as shown herein.

FIG. 22. Schematic of chimeric proteins fusing different HD-Zip domains

Chimerical proteins fusing the HD-Zip domain of one protein with the carboxy-terminal domain of another were used to transform Arabidopsis plants, obtain homozygous lines and analyze phenotypes, especially regarding known characteristics conferred by wt proteins HAHB1, as compared, for example, to the known characteristics and effects of HAHB4. Left side: names of the constructs. CI and CII represent the two carboxy-terminus conserved motifs of HAHB1. C-ter indicates the carboxy-terminus of HAHB4 which is different from HAHB1 or its homologues.

FIG. 23. Alignment of different HAHB1 regions vs non-redundant protein DB (Blastp algorithm)

The bars in the graph represent the % of identity (ID) or similarity (SIM) between certain regions of HAHB1 and the most similar protein in that region (white), ATHB13 (patterned) and ATHB23 (black) as it has been calculated by the Blatp algorithm. The different segments compared are: complete HAHB1 sequence, the HD-Zip domains, the whole COO terminal (CICII), the first motif isolated (CI) and the second motif isolated (CII). The Blastp algorithm calculates these percentages taking the region with highest homology. This is shown in the table below.

Alignment Summary

| | HAHB1 REGION | | | | | |
|---|---|---|---|---|---|---|
| | Complete | | | | | |
| | 313 | | HDZip | | CICII | |
| | | Similar- | 100 | | 122 | |
| | Identity | ity | Identity | Similarity | Identity | Similarity |
| Most | 224/314 | 47/314 | 92/100 | 97/100 | 79/121 | 84/121 |
| similar | (71%) | (78%) | (92%) | (97%) | (65%) | (69%) |
| ATHB13 | 192/322 | 222/322 | 86/100 | 91/100 | 56/124 | 68/124 |
| | (59%) | (68%) | (86%) | (91%) | (45%) | (54%) |
| ATHB23 | 162/316 | 199/316 | 76/100 | 91/100 | 50/121 | 59/121 |
| | (51%) | (62%) | (76%) | (91%) | (41%) | (48%) |

FIG. 24. Apoplastic proteins of transgenic (35S:PR2) and wild type plants

SDS-PAGE showing the apoplastic proteins obtained from non-acclimated WT and transgenic (35S:PR2) plants. The samples were isolated from 25-day-old plants grown in normal conditions. The arrow indicates the band differentially expressed. Apoplastic proteins from the WT and transgenic plants grown in normal conditions were isolated and analyzed by SDS-PAGE. As it can be observed, although the plants were not subjected to cold stress, the PR2 expressed is present in the cellular apoplast in the transgenic genotype. The molecular weight of this band is coincident with the expected for PR2, but sequence determination was not carried out yet.

FIG. 25. Putative PR2, PR4 and glucanase are upregulated in transiently transformed soybean and Nicotiana tabacum leaf disks with the constructs 35S:HAHB1 and 35S:ATHB13.

Transcript levels of different response genes were measured by qRT-PCR; ACTIN genes (ACTIN2 plus ACTIN8) were used as internal controls and standard deviations were calculated from at least three independent experiments; the measured genes for sunflower are chitinase (tc18434), HASAG21 (tc19654), HAZAT10 (tc16546) and HADREB (tc23839) while for soybean, they were GMPR2 (AK285952.1), GMPR4 (AK246040.1) and GM-glucanase (AY461847.1) and for tobacco, NTPR2 (EU867448.1) and NTPR4 (S72452.1)

FIG. 26. Homozygous plants expressing the transcription factor HAHB1 are more tolerant to Pseudomonas infection.

26a: Plants infected with Pseudomonas are dying (wilted area is shown with an arrow) whilst transgenic plants are healthy and green. 26b is a photographs of the plants two days after the infection. In this photograph it can be seen that transgenic plants do not present necrosis, visualized by brighter coloured areas, while WT plants do. 26c is a photograph taken after staining with Evans blue (which colours the necrosis tissue). In this figure also it can be appreciated a difference between genotypes: WT plants are stained (speckled areas as marked by arrow) while transgenic are not.

FIG. 27. Homozygous Arabidopsis plants expressing 35S-β glucanase and PR2.

35S-β glucanase (At4g16260) is one of the target genes of HAHB1 identified in the microarray experiments and plants expressing 35S-PR2 are more resistant to freezing conditions. 121 designates wild type (14% survivors in freezing conditions), 35S-β glucanase (62%), 35S-PR2 (42%). Control plants exhibit severe damage visualized as a marked wilting of the whole plant after a freezing treatment while transgenics present a greater percentage of healthy tissue, being higher in 35S:glucanase than in the 35S:PR2 genotype.

FIG. 28. PR4 (three of five independent lines shown) confers freezing tolerance when it is over and ectopically expressed in transgenic Arabidopsis plants.

Plants were grown in control conditions during 25 days and then treated 2 hours at −8° C. in vermiculite/perlite. The plants were then placed in normal conditions for 6 days for recovery before being photographed. Control plants exhibit severe damage visualized as a marked wilting of the whole plant after a freezing treatment while transgenics (35S:PR4) present a greater percentage of healthy tissue. Transgenic plants were F2 (heterozygous). Percentage of survivors of each genotype after treatment:

| Genotype | Survivors % |
|---|---|
| WT | 31 |
| PR4-17 | 75 |
| PR4-19 | 75 |
| PR4-6 | 92 |
| PR4-10 | 75 |
| PR4-23 | 50 |

FIG. 29. Membrane stability in wt and transgenic plants expressing PR2 (line 8, 16 and 18) measured as conductivity of released saline.

The lower relative conductance indicate greater membrane stability in 35S:PR2 plants compared with WT plants after a freezing treatment. The x axis shows a treatment of “1 hour at −8° C.”.

FIG. 30. Membrane stability in wt and transgenic plants expressing glucanase measured as conductivity of released saline.

The leaching technique was carried out essentially as described by Sukumaran and Waiser (1972). Plants were grown for 20 days in standard conditions and then irrigated with 50 mM NaCl (1 l in a 45×45 cm tray). One week after this, the plants were irrigated with 150 NaCl and one more week later with 200 mM NaCl. One day after each saline irrigation, six leaves from each plant were excised and exhaustively washed with distilled water. After that the leaves were placed in 15 ml of double-distilled deionized water with continuous agitation in a water bath at 25° C. for 3 hr. After decantation the aqueous extract conductance (C1) was measured. Then, the leaves were placed in a 65° C. water bath for 16 hours with continuous agitation and one additional hour at 25° C. prior to the measurement of the solution conductance (C2). The real conductance was calculated as the ratio between C2/C1 (L=C1/C) and used as index of injury. L values higher than 0.5 indicate a severe injury. The Y axis indicates “relative conductance”. The lower relative conductance in 35S:H1 plants indicates greater membrane stability than in PrH1:H1 and WT plants after a one hour freezing treatment. In control conditions or after two hours of freezing treatment, there is no difference between genotypes.

FIG. 31. Glucanase transgenic plants are more tolerant to drought stress

25 day old plants were subjected to drought stress by not watering the plants for 7 days. The photographs were taken five days after re-watering. 5b, 14a and 27a are lines carrying the glucanase transgene. WT plants (first column of plants left hand side) are more severely damaged than 35S:glucanase plants (second to third columns), as the wilting in their leaves demonstrates. Percentage of survivors of each genotype after treatment:

| Genotype | Survivors % |
|---|---|
| Wt | 20 |
| 5B | 46 |
| 14A | 62 |
| 27A | 69 |

FIG. 32. Homozygous plants overexpressing PR2 are more tolerant to drought stress

This assay was performed essentially as the one described in FIG. 31, but with the genotype 35S:PR2 vs WT plants. The wilting in the aerial portion of WT plants (first column of plants on the left hand side) shows that they are significantly more affected by drought than 35S:PR2 plants (second to third columns: lines 8C, 16B, 18B), which display healthier tissues.

Percentage of survivors of each genotype after treatment:

| Genotype | Survivors % |
|---|---|
| Wt | 23 |
| 8C | 62 |
| 16B | 44 |
| 18B | 50 |

FIG. 33. Seedlings treated with ACC, ethylene precursor, were evaluated for the development of the triple response.

The presence or absence of the apical hook was quantified and the resulting proportions used to compare the degree of response in each line. 35SCaMV:HAHB4 Arabidopsis lines were almost unresponsive to ethylene. H4WCT, H1WCT and H4H1 showed an intermediate response. Finally, HAHB1 and WT were highly responsive. X axis: lines, Y axis: plants without an apical hook (proportion).

FIG. 34. ATHB13 expression kinetics in plants subjected to −8° C.

Kinetics of induction of ATHB13 in 21-day-old seedlings placed at −8° C. during 3 hours. Samples were taken at 0, 30, 60, 120 and 180 minutes. Transcript levels of ATHB13 in 21-day-old leaves placed at −8° C. were determined by quantitative RT-PCR and standard deviations calculated from three independent samples in which actin transcripts (ACTIN2 plus ACTIN8) were used as internal controls; all the values are normalized with respect to the value measured at time 0, arbitrarily assigned a value of one. The Y axis shows fold induction.

### DETAILED DESCRIPTION OF THE PREFERRED EMBODIMENTS OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, which are within the skill of the art. Such techniques are explained fully in the literature.

The term stress/stress tolerance as used herein includes abiotic and biotic stress. Said stress/stress tolerance is preferably selected from freezing, low temperature, chilling, drought, high salinity and/or invasion of pathogens. As shown herein, the transgenic plants show increased/enhanced tolerance to these types of stresses. The tolerance can be measured as shown in the examples. Tolerance is increased compared to wild type (wt) plants. The increase can be at least two-fold up to 10 fold or more.

In a one aspect, the invention relates to an isolated nucleic acid sequence comprising a nucleic acid sequence of SEQ ID. No. 1, a functional fragment, part or a functional variant thereof. In one embodiment, the isolated nucleic acid sequence comprises or consists of a nucleic acid sequence of SEQ ID. No. 1.

The term “functional part or functional variant of HAHB1” as used herein refers to a variant gene sequence or part of the gene sequence which retains the biological function of the full non-variant sequence, i.e. confers stress tolerance when expressed in a transgenic plant. Specifically, the variant may be a chimeric sequence that encodes for the C-terminus of HAHB1 as described herein (or to CI and/or CII of HAHB1 or the consensus sequence), for example coupled to the N-terminus of another HD-Zip I family member. The term “variant” may also refer to a sequence that encodes a peptide/protein sequence that is homologous to HAHB1 and which shows homology in the HD and LZ domains and also in the C-terminal domains (in particular to CI and CII as explained below). A functional variant also comprises a variant of HAHB1 which is substantially identical, i.e. has only slight sequence variations, for example in non conserved residues, to the HAHB1 and HAHB1 sequences as shown herein and confers stress tolerance. A functional part may be the sequence encoding for the CI and/or CII motif.

The inventors have also shown that the HAHB1 promoter as defined in SEQ ID. No. 1 is effective in sensing stressful conditions and can be used to confer stress-induced gene expression on a transgene, for example under chilling, freezing, low salinity or drought conditions. Thus, the invention also relates to a stress inducible promoter comprising or consisting of a nucleic acid sequence of SEQ ID. No. 1, a functional fragment or a functional variant thereof. The promoter is useful for controlling transgene expression of transgenes in transgenic plants transformed with a gene under the control of said promoter. Accordingly, the invention also relates to a vector comprising a gene construct comprising a nucleic acid sequence of SEQ ID. No. 1, a functional fragment or a functional variant thereof and to the use of a sequence as defined in SEQ ID No. 1, a functional fragment or a functional variant thereof as a stress inducible promoter. Furthermore, the invention relates to a method for conferring stress induced gene expression in a plant wherein said method comprises transforming a plant with an expression cassette comprising a nucleic acid sequence of SEQ ID No. 1, a functional fragment or a functional variant thereof, operably linked to a gene sequence of interest for expression.

Any gene sequence of interest may be operatively associated in this manner to achieve such induced expression on exposure of plants bearing the construct to appropriate stress conditions selected from freezing, low-temperature, chilling drought and/or conditions of high salinity.

In a further aspect, the invention relates to an isolated nucleic acid sequence comprising or consisting of a nucleic acid sequence of SEQ ID. No. 7. In another aspect, the invention relates to an isolated nucleic acid sequence comprising or consisting of a nucleic acid sequence of SEQ ID. No. 2. The invention also relates to an isolated polypeptide sequence comprising or consisting of a sequence of SEQ ID. No. 5.

The invention further relates to vectors which comprise gene constructs encoding for a protein that confers stress tolerance in plants. Specifically, the invention relates to a vector comprising a gene construct encoding for the HAHB1 protein comprising or consisting of SEQ ID No. 5, a functional part or variant thereof. In one embodiment, the vector comprises a gene construct encoding for the HAHB1 protein comprising or consisting of SEQ ID No. 5.

In a further aspect, the invention relates to a vector comprising a gene construct comprising a nucleic acid sequence of SEQ ID. No 2, 6 or 7, a functional part or functional variant thereof. In particular, the sequence comprises or consists of nucleic acid SEQ ID. No. 6 or 7. In one embodiment, a nucleic acid sequence of SEQ ID. No 2, 6 or 7, a functional part or functional variant thereof is operably linked to a promoter sequence. For example, the vector comprises an expression cassette wherein a nucleic acid sequence of SEQ ID. 6 is operably linked to a promoter. In another embodiment, a nucleic acid sequence of SEQ ID. 7 is operably linked to a promoter.

The promoter used in the gene constructs of the vectors described above may be the endogenous HAHB1 promoter comprising SEQ ID No. 1, a functional part or functional variant thereof. For example, the vector comprises an expression cassette wherein a nucleic acid sequence of SEQ ID. 6 or 7 is operably linked to the HAHB1 promoter comprising a nucleic acid sequence of SEQ ID No. 1. In another embodiment, the promoter may be a promoter that drives constitutive overexpression of a gene. Overexpression according to the invention means that the transgene is expressed at a level that is higher than expression driven by its endogenous promoter. For example, overexpression may be carried out using a strong promoter, such as the cauliflower mosaic virus promoter (CaMV35S), the rice actin promoter or the maize ubiquitin promoter or any promoter that gives enhanced expression. A stress-inducible promoter, such as the RubisCO small subunit promoter may also be used. This list is not considered limiting as the skilled person will be able to select a suitable promoter.

For example, the vector comprises an expression cassette wherein a nucleic acid sequence of SEQ ID. 6 or 7 is operably linked to the CaMV35S promoter. Alternatively, enhanced or increased expression can be achieved by using transcription or translation enhancers or activators and may incorporate enhancers into the gene to further increase expression. Furthermore, an inducible expression system may be used, such as a steroid or ethanol inducible expression system. Also envisaged is ectopic expression, i.e. gene expression in a tissue in which it is normally not expressed.

In another aspect, the invention relates to a host cell transformed with a vector or a gene sequence as described herein. Specifically, the invention relates to a host cell expressing a protein of SEQ ID NO. 5, a functional part or functional variant thereof. In a preferred embodiment, the cell is a plant cell. The plant cell may be a cell of a monocot or dicot plant as further defined herein.

We demonstrate herein that HAHB1 confers stress tolerance, for example freezing tolerance, to transgenic plants via the induction of antifreeze protein (AFP) biosynthesis, which inhibits ice crystal formation.

Therefore, in another aspect, the invention relates to a transgenic plant transformed with a vector as described herein or transformed with a gene sequence as described herein. Thus, the invention relates to a transgenic plant expressing or overexpressing a gene encoding for the sunflower HAHB1 protein or a variant or functional part thereof. Preferably, the HAHB1 protein comprises or substantially consists of a sequence as defined in SEQ ID NO. 5, a functional part or functional variant thereof. Thus, in one embodiment, the transgenic plant expresses or overexpresses a gene encoding for the HAHB1 protein as defined in SEQ ID NO. 5. For example, the plant may be transformed with a gene construct comprising a nucleic acid sequence of SEQ ID No. 6 or 7, a functional part or functional variant thereof. The nucleic acid comprising a sequence of SEQ ID No. 6 or 7 may be under the control of a promoter as defined above. In one embodiment, the nucleic acid comprising a sequence of SEQ ID No. 6 or 7 is under the control of the HAHB1 promoter comprising a nucleic acid sequence of SEQ ID No. 1, a functional part or functional variant thereof. In another embodiment, the nucleic acid comprising a sequence of SEQ ID No. 6 or 7 is under the control of the CaMV35S promoter. As explained below, the gene sequence may also be a gene sequence that encodes a homolog of HAHB1 (with high homology in the HD, LZ and CICII domains), such as ATHB13, or a chimeric construct that comprises the C-terminal domain of HAHB1. The plant is characterised in that it is more tolerant to stress conditions compared to its wild type counterpart, specifically to a stress selected from freezing, low temperature, chilling, drought, high salinity and/or invasion of pathogens. In one embodiment, the stress is freezing.

Thus, in one embodiment, the invention relates to a method for producing a stress tolerant plant or enhancing stress tolerance of a plant comprising transforming a plant with a nucleic acid sequence encoding for ATHB13 comprising a sequence as shown in SEQ ID NO. 64 or a functional variant or part thereof. Thus, the uses and methods as claimed in respect of SEQ ID NO. 2, 6 and 7 also apply to SEQ ID NO. 64. Another example that can be used is ATHB23, a gene that shares high homology with HAHB1.

The transgenic plant thus obtained is characterised in that it shows enhanced stress tolerance compared to a control plant. The control plant is preferably a wild type plant.

In another aspect, the invention relates to a method for producing a stress tolerant plant comprising transforming a plant with a nucleic acid sequence as described herein or a vector as described herein. The invention also relates to a method for increasing stress tolerance in a plant comprising transforming a plant with a nucleic acid sequence as described herein or a vector as described herein.

In one embodiment of these methods, the transgene directs the expression of the HAHB1 protein comprising a sequence of SEQ ID No. 5. For example, transformation may be using a nucleic acid comprising a sequence of SEQ ID No. 6 or 7. This sequence may be under the control of the HAHB1 gene promoter comprising a nucleic acid sequence of SEQ ID No. 1, a functional part or functional variant thereof or under the control of the CaMV35S promoter. The transgene may also direct expression of a part of the HAHB1 protein (for example the C-terminus fused to the N-terminus of another HD-Zip protein) or of a homolog of HAHB1, for example ATHB13.

In connection with the foregoing aspects of the invention, stress tolerance is selected from tolerance to abiotic or biotic stress. Abiotic stress is selected from freezing, low-temperature, chilling, drought and/or conditions of high salinity. Biotic stress is stress caused by pathogenic organisms, such as bacterial or fungal pathogens (see example 16 and FIG. 26).

In another aspect, the invention relates to a plant obtainable or obtained by a method as described herein.

In another aspect, the invention relates to a use of a nucleic acid sequence or use of a vector as defined herein in conferring increasing stress tolerance in a plant. In one embodiment, the sequence used is a nucleic acid comprising a sequence of SEQ ID No. 6 or 7. This sequence may be under the control of the HAHB1 promoter comprising a nucleic acid sequence of SEQ ID No. 1, a functional part or functional variant thereof or under the control of the CaMV35S promoter. Said stress tolerance is selected from tolerance to abiotic or biotic stress. Abiotic stress is selected from freezing, low-temperature, chilling tolerance, drought and/or conditions of high salinity. Biotic stress is invasion by a pathogen, for example Pseudomonas.

As shown herein, the plant into which a vector or sequence as defined herein is introduced may be Arabidopsis. The inventors have shown that the HAHB1 gene sequence from sunflower can direct the expression of the HAHB1 protein in Arabidopsis. Moreover, the inventors have shown that transgenic expression of 35S:HABH1 and expression of 35S:ATHB13 respectively in other plants or plant tissue results in the upregulation of the expected target genes, thus providing evidence that both HABH1 and ATHB13 are effective in exogenous plant hosts and that transgenic expression of HABH1 and ATHB13 has universal application in genetically manipulating plants. The skilled person thus would know that the invention is not limited to Arabidopsis, soybean or tobacco which are used as non-limiting examples in the experiments herein. The skilled person would know that any monocot or dicot plant can be used. A dicot plant may be selected from the families including, but not limited to Asteraceae, Brassicaceae (eg Brassica napus), Chenopodiaceae, Cucurbitaceae, Leguminosae (Caesalpiniaceae, Aesalpiniaceae Mimosaceae, Papilionaceae or Fabaceae), Malvaceae, Rosaceae or Solanaceae. For example, the plant may be selected from lettuce, sunflower, Arabidopsis, broccoli, spinach, water melon, squash, cabbage, tomato, potato, capsicum, tobacco, cotton, okra, apple, rose, strawberry, alfalfa, bean, soybean, field (fava) bean, pea, lentil, peanut, chickpea, apricots, pears, peach, grape vine or citrus species. In one embodiment, the plant is oilseed rape.

Also included are biofuel and bioenergy crops such as sugar cane, oilseed rape/canola, linseed, and willow, poplar, poplar hybrids, switchgrass, Miscanthus or gymnosperms, such as loblolly pine. Also included are crops for silage (eg forage maize), grazing or fodder (grasses, clover, sanfoin, alfalfa), fibres (e.g. cotton, flax), building materials (e.g. pine, oak), pulping (e.g. poplar), feeder stocks for the chemical industry (e.g. high erucic acid oil seed rape, linseed) and for amenity purposes (e.g. turf grasses for golf courses), ornamentals for public and private gardens (e.g. snapdragon, petunia, roses, geranium, Nicotiana sp.) and plants and cut flowers for the home (African violets, Begonias, chrysanthemums, geraniums, Coleus spider plants, Dracaena, rubber plant).

A monocot plant may, for example, be selected from the families Arecaceae, Amaryllidaceae or Poaceae. For example, the plant may be a cereal crop, such as wheat, rice, barley, maize, oat, sorghum, rye, onion, leek, millet, buckwheat, turf grass, Italian rye grass, switchgrass, Miscanthus, sugarcane or Festuca species.

Preferably, the plant into which a sequence or vector of the invention is introduced is a crop plant. By crop plant is meant any plant which is grown on a commercial scale for human or animal consumption or use or other non-food/feed use.

Preferred plants are maize, tobacco, wheat, rice, oilseed rape, sorghum, soybean, potato, tomato, barley, pea, bean, field bean, cotton, lettuce, broccoli or other vegetable brassicas or poplar.

A sequence or vector described herein encoding for the HAHB1 protein is introduced as a transgene into the plant. This can be carried out by various methods as known in the field of plant genetic engineering, for example using transformation with Agrobacterium or particle bombardment.

The gene may be an exogenous gene, such as sunflower HAHB1, expressed in a different plant species. Alternatively, the invention also relates to using an endogenous gene expressing a HAHB1 homologue, i.e. a gene encoding for a homologue of HAHB1 that is endogenous to the plant in which it is introduced and expressed or overexpressed. As explained elsewhere herein, the homologous gene shows high sequence similarity in the HD and LZ domains, but also in the C-terminal domains (CI and CII) as the C-terminal domains appear crucial in conferring HAHB1 function.

We describe measurement of several select parameters in plants transformed with a construct bearing the HAHB1 gene under the control of a constitutive promoter, such as the 35S promoter, operatively linked to the coding sequence of the HAHB1 protein, or a suitable variant, analogue, homologue or orthologue thereof, referred to generally herein as HAHB1, to produce a construct entitled 35S:HAHB1, or under the control of the promoter of the HAHB1 gene, a suitable variant, analogue, homologue or orthologue thereof, generally referred to herein as promHAHB1:HAHB1. The comparison, using those parameters, of these plants with plants transformed with a control construct, e.g. pBI 121, is described in further detail in the Experimental section below, and referred to herein as “WT”. The comparison confirms that the increase in freezing tolerance observed in the plants comprising the HAHB1 construct, under control of either promoter, is due to the action of the induced AFPs, and that this ultimately helps to conserve membrane stability and chlorophyll content as well as to inhibit ice crystal formation and damage in the recombinant plants.

It has been reported previously that it is common to observe developmental and morphological penalties in transformed plants bearing transcription factors controlled by constitutive promoters such the CaMV 35S as transgenes (Arce et al., 2008 and references therein). These penalties are probably caused by the metabolic costs generated by the activation of specific protein biosynthesis, unnecessary in normal growth conditions. In this sense, HAHB1 seems to delay very slightly the developmental rate in the earlier developmental stages of plants transformed with the 35S:HAHB1 construct, but this characteristic almost completely disappears later in the transformed plant's life cycle. No morphological or developmental negative characteristics were observed in transgenic plants transformed with the construct promHAHB1:HAHB1, indicating that in these plants the gene is expressed at a very low level in the absence of environmental stress. Apparently these transcript levels are not enough to produce the high metabolic costs but sufficient to achieve the desired freezing tolerance response.

It is important to distinguish the difference between chilling and freezing conditions because the consequences and the responses triggered by the plants are not the same in these two distinct stresses. Prolonged freezing causes tissue death and, ultimately, plant death while prolonged chilling results in plant acclimation and developmental arrest. In Arabidopsis plants, whether WT or transgenic according to this invention, incubated for several months at 4° C., the plants survive when placed in normal conditions. Subsequent exposure of these acclimated plants to freezing conditions demonstrates enhanced survival of the acclimated plants as compared to non-acclimated plants. In spite of not being able to detect a difference in survival in chilling conditions between the acclimated WT and acclimated HAHB1-transformed plants we could measure a significant difference, both in chlorophyll content and in membrane stability between genotypes, thus indicating a better tolerance achieved by the transgenic genotype.

On the other hand, freezing treatments detected a greater percentage of survival, both in vegetative and reproductive stages, for the HAHB1-transformed plants as compared to WT plants, reinforcing the results obtained during chilling. Together, the observations confirm that transformation with HAHB1 confers both freezing tolerance and chilling tolerance to plants.

As well as freezing, or chilling, drought or high salt concentrations, for example, also cause stress to plants. However, the tolerance of plants to different types of abiotic stresses is not necessarily conferred through related mechanisms and indeed occurs via different signal transduction pathways. Thus, it cannot be expected that a gene that confers, when expressed, one type of stress, could also confer a different type of stress. Notably and unexpectedly, we demonstrate herein that HAHB1 acts to enhance tolerance responses in plants to all three of these different stress factors—freezing, drought and high salt. HAHB1 transgenic plants are more tolerant to drought and high salinity than the non-transformed controls, indicating that the HD-Zip protein is somehow concomitantly acting in several abiotic stress responses, at least when it is constitutively expressed. Microarray data confirms this, since several genes previously described as involved in abiotic stress tolerance, such DREB, are strongly induced in transgenic plants ectopically and constitutively expressing HAHB1.

Moreover, as shown in the examples, HAHB1 transgenic plants are more tolerant to invasion by pathogens, for example Pseudomonas.

Tolerance responses are conserved among species. In winter rye, one of the more characterized species with respect to the freezing response, antifreeze proteins homologous to those detected in the Arabidopsis microarray, are ultimately responsible for this tolerance. On the other hand, although there is limited genomic data available for sunflower, we were able to identify some genes (homologous to those detected in the microarray) showing higher expression levels in transiently transformed sunflower leaves. Acclimation assays indicate a consistent result in that apoplast protein patterns change both in Arabidopsis and sunflower when the plants are placed at 4° C. In the Experimental Procedures section herein, we provide data from recrystallization experiments which show that transgenic plant apoplast extracts are more efficient at preventing ice crystal formation than apoplast extracts from control plants.

We also show that Arabidopsis PR proteins, thought to function in anti-pathogen responses, act in the antifreeze response. Plants overexpressing HAHB1 and separately, PR2, PR3 and glucanase were tested in freezing conditions and these genotypes were more tolerant to freezing than control (wt) plants. Without wishing to be bound by theory, it is possible, on the one hand, that a group of antifreeze proteins may be cooperatively acting to achieve an optimal tolerance and only if HAHB1 is present are all of them expressed. On the other hand, based on the data presented herein, some of these PR proteins may be able to confer freezing tolerance by themselves if they are highly expressed. As shown in example 10 and FIGS. 27 to 32, overexpression of PR2, PR4 and β-glucanase, confers tolerance to freezing conditions compared to the wild type. Moreover, tolerance to increased salinity was also observed. Thus, expressing PR2, PR4 and β-glucanase in a transgenic plant can lead to increased stress resistance. Expression may be from the endogenous promoter or any other promoter defined herein, for example a promoter suitable for overexpression.

Thus, in another aspect, the invention relates to a method for producing a stress tolerant plant or enhancing stress tolerance of a plant comprising transforming a plant with a nucleic acid sequence encoding for PR2. The sequence may comprise SEQ ID NO. 66, a homologue or variant thereof. Said tolerance is preferably freezing tolerance or drought tolerance. A plant obtained by this method is also within the scope of the invention. Also within the scope of the invention is the use of a nucleic acid sequence encoding for PR2 in increasing/conferring stress tolerance.

In a further aspect, the invention relates to a method for producing a stress tolerant plant or enhancing stress tolerance of a plant comprising transforming a plant with a nucleic acid sequence encoding for glucanase. The sequence may comprise SEQ ID NO. 65, a homologue or variant thereof. Said tolerance is preferably freezing tolerance or drought tolerance. A plant obtained by this method is also within the scope of the invention. Also within the scope of the invention is the use of a nucleic acid sequence encoding for glucanase in increasing/conferring stress tolerance.

In a further aspect, the invention relates to a method for producing a stress tolerant plant or enhancing stress tolerance of a plant comprising transforming a plant with a nucleic acid sequence encoding for PR4. The sequence may comprise SEQ ID NO. 67, a homologue or variant thereof. Said tolerance is preferably freezing tolerance or drought tolerance. A plant obtained by this method is also within the scope of the invention. Also within the scope of the invention is the use of a nucleic acid sequence encoding for PR4 in increasing/conferring stress tolerance.

In another aspect, the invention relates to a method for identifying a nucleic acid sequence which confers stress tolerance when introduced into a plant, which comprises using a nucleic acid sequence comprising a sequence of SEQ. ID. NO: 1, 2, 6, 7, or a part thereof or a sequence encoding for a sequence having homology to the sequence of SEQ. ID. NO. 11, 12 or 14, or a nucleic acid encoding all or a selected part of SEQ. ID. NO: 8 or 13 or a nucleic acid encoding all or a selected part of SEQ. ID. NO: 5, to probe a plant genome or plant genomic clones in a library. The sequences can also be used to probe an electronic library, thus identifying sequences with homology using bioinformatics.

The invention also relates to an isolated nucleic acid sequence obtained or obtainable by the method described above.

In another aspect, the invention relates to nucleic acid sequence homologous to the HAHB1 sequence as defined in SEQ ID NO. 6 or 7 wherein said gene sequence is capable of conferring stress tolerance when introduced and expressed in a plant.

Preferably, said homologous nucleic acid sequence shows at least 80%, preferably at least 90%, more preferably at least 95% homology to the HAHB1 sequence comprising SEQ ID NO. 6 or 7.

The homologous nucleic acid sequence is characterised in that it encodes a protein that has in its N terminal region a homeodomain with homology to the consensus as shown in SEQ ID No. 14.

The sequence also comprises a leucine zipper. The leucine zipper is already defined, i.e., in the CDD database, in which it is identified with this name: PSSM Id: 121415.

Moreover, the homologous nucleic acid sequence is characterised in that it encodes a protein that has a C-terminal sequence with homology to the C-terminus sequence of HAHB1 as shown in SEQ ID NO. 8. Furthermore, the homologous nucleic acid sequence is characterised in that it encodes a protein that has a C-terminal sequence comprising a sequence with homology to the consensus sequence as shown in SEQ ID NO. 11 and/or 12. Preferably, said homology is at least 80%, preferably at least 90%, more preferably at least 95% homology to the HAHB1 sequence comprising SEQ ID NO. 11.

From the information provided herein, a skilled person will appreciate the presence of conserved domains and will know that on the basis of the sequence information provided, primers can also be designed to aid the identification of homologous.

In addition to the protein sequence homology and the biological function of conferring stress tolerance when expressed in a plant by transgene technology, a homologous sequence can also be identified because it shows similarity to HAHB1 in respect of the expression pattern directed by its promoters and the behaviour in sucrose.

Due to the detailed sequence information available, it is also possible to identify homologous sequences from existing databases using bioinformatics. Once the homologue sequences has been thus identified, it is possible to design oligonucleotides to amplify the respective cDNAs using RNA isolated from the chosen plant in a condition in which the gene is expressed. After amplification, the DNA segment can be cloned in a suitable vector to be checked by sequence determination (multicopy) and subcloned in a vector suitable for plant transformation directed by a selected promoter. If the promoter of the homologue is wanted, it is necessary to amplify it by PCR or alternatively to isolate the whole gene from a genomic library. Isolation from a genomic library is obliged when the whole sequence is unknown and only a partial segment is known to construct a probe. In the same way the cDNA can be isolated from a cDNA library if such library and a suitable probe are available. The gene thus identified can then be used in the method described below.

In another aspect, the invention relates to a method for producing a stress tolerant plant or enhancing stress tolerance of a plant comprising transforming a plant with a homologous nucleic acid sequence as described above.

The classification of HD-Zip proteins into four subfamilies is supported by the following four distinguishing characteristics: conservation of the HD-Zip domain determining DNA binding specificities, gene structures, additional conserved motifs and functions.

Members of the HD-Zip family exhibit a LZ motif just downstream from the HD motif. The two motifs are present in transcription factors belonging to other eukaryotic kingdoms, but their association with each other in a single protein is unique to plants. The HD is responsible for the specific binding to DNA while the LZ acts as a dimerization motif. HD-Zip proteins bind to DNA as dimers, and the absence of the LZ absolutely abolishes their binding ability, indicating that the relative orientation of the monomers, driven by this motif, is crucial for an efficient recognition of DNA.

In Arabidopsis, subfamily I is composed of seventeen members (ATHB1/HAT5, 3/HAT7, 5, 6, 7, 12, 13, 16, 20, 21, 22, 23, 40, 51, 52, 53, 54). HD-Zip I subsets of genes (in Arabidopsis) share their intron/exon distribution in accordance with their phylogenetic relationships. The molecular weight of the encoded proteins is about 35 kDa and exhibit a highly conserved HD and a less conserved LZ. No other similarity or the presence of additional conserved motifs has, to our knowledge, been described.

A full in vitro description, consisting of PCR-assisted binding site selection and footprinting assays, determined that all the proteins encoded by HD-Zip I genes and tested, recognize, as dimmers, the pseudopalindromic sequence CAAT(A/T)ATTG (Ariel et al., 2007).

Aligning the 15 most homologous Arabidopsis HD-ZIP subfamily I proteins, it is possible to obtain a relatively good consensus for the homeodomain, but not for the leucine zipper. The definition of the latter depends on a conservation of leucines and certain other residues with special attention to relative position, so homology percentages tend to be very low (the conservation is mainly structural defined by the positions of the leucines).

In an alignment of 15 Arabidopsis thaliana HD-Zip class I proteins, the percentages are much higher for the homeodomain than for the leucine zipper domain.

Expression of the HD-Zip class I genes that have been characterized indicates that its expression is regulated by external factors like drought, extreme temperatures, osmotic stresses and illumination conditions, and is specific to different tissues and organs of the plant. Their role as transcription factors is related to developmental events in response to such environmental conditions, particularly those in which abiotic factors generate stress but not necessarily conferring tolerance to such stresses. Nor, for those factors which do confer tolerance, can it be predicted, a priori, to which stress they will confer resistance.

Different subsets of Arabidopsis HD-Zip I genes that bear a close phylogenetic resemblance exhibit common organ expression patterns and are responsive to the same environmental factors.

ATHB1, the first isolated member, acts as a mediator in the leaf cell fate determination, whereas ATHB16 is involved in blue-light perception signalling. Another group of genes was proposed to be involved in ABA-related and abiotic stress responses. Evidence was obtained from expression studies and transgenic plants, indicating that ATHB7, 12, 5 and 6 are up or down regulated by water deficit conditions and/or externally applied ABA. Under the effect of these stimuli, HD-Zip I genes behave as developmental and growth regulators. The sunflower HAHB4 gene, for its part, confers drought tolerance to transgenic Arabidopsis plants when it is expressed under the control of constitutive or drought-inducible promoters, while ATHB7 and ATHB12 do not, even though they are the more related genes in Arabidopsis, except for at the C-terminus. Since the sunflower genome sequence is not available, and considering that HAHB4 does not behave as do ATHB7 or 12, it is most likely that they are not orthologous genes even if they exhibit a high sequence homology and are regulated by the same external factors.

Overexpression of ATHB3, 13, 20 or 23 suggests that these genes are involved in the regulation of cotyledon and leaf development, even though ATHB13 and ATHB23 are the Arabidopsis genes presenting the highest homology with HAHB1.

HD-Zip I genes have evolved by a series of gene duplications to a considerable complexity, resulting in subsets of paralogous genes which share intron/exon distribution, amino acid sequences and expression patterns. Ectopic expression of each HD-Zip I subset of genes provokes different phenotypic effects.

The inventors investigated why HD-Zip I proteins, which all bind to the same DNA sequences (a feature determined by helix III of the HD domain) and have similar expression patterns, nevertheless participate in different signal transduction pathways and even exert diverse functions.

We have shown herein that a comparison of each HD-Zip member shows a high degree of conservation in the carboxy-terminus region of proteins belonging to different species (see alignment provided as FIG. 21 herein).

Subfamily I can be divided into several groups according to the conservation in the carboxy terminus domain. All the members share homology in the HD-Zip domain, but when the alignment with the whole encoded proteins is performed, several groups can be visualized (phylogenetic tree) that differ between them in the carboxy terminus.

Notwithstanding the high degree of conservation presented in this region, no similarity with any other known motif has been detected, despite a deep bioinformatic analysis. HAHB1 shares this similarity with proteins of varied and distant species. Without wishing to be bound by theory, the inventors believe that the C terminal region provides a disordered protein-protein interaction domain, and the interaction of this domain with other diverse proteins determines the function of the protein.

The inventors have shown herein that without the HD-Zip domain, the HAHB1 COO terminus does not function autonomously. They have also shown that the HD-Zip of HAHB4 (with its COO terminus deleted) fused to the COO terminus of HAHB1, appears to behave like HAHB1. All the HD-Zip I members characterized up to now bind the same DNA sequence. Therefore, it is believed that the HAHB1 COO terminus fused to the HD-Zip region of any subfamily I HD-Zip domain acts as a functional HAHB1.

From the experiments shown herein, it can be seen that it is the C-terminal region (CICII) that confers protein function. Diversity in the C terminus of the members of the HD-Zip I family thus seem to explain the different functions and developmental effects of these proteins. Chimeric constructs which comprise the HAHB1 COO terminus fused to the HD-Zip region of any subfamily I HD-Zip domain acts as a functional HAHB1 can therefore also be use din the methods for conferring increased abiotic and biotic stress tolerance as described herein.

In such a chimeric construct, the N-terminal region is characterised in that it comprises a homeodomain with homology to the consensus as shown in SEQ ID NO. 14 (which was obtained from subfamily I), associated in its C terminus to a leucine zipper. Moreover, this conserved homeodomain is able to bind the palindromic sequence CAAT(A/T)ATTG which is characteristic for this subfamily and differs from the sequences bound by members of other subfamilies.

Therefore, in another aspect, the invention relates to an isolated nucleic acid sequence comprising or consisting of a sequence as defined in SEQ ID No. 8, 9, 10, 11, 12 or 13. Also within the scope of the invention are vectors comprising such sequences.

In another aspect, the invention relates to a chimeric gene construct comprising a nucleic acid sequence encoding for the N-terminal sequence of an HD Zip protein, a part thereof or a sequence comprising a N-terminal consensus motif of subfamily I, operatively associated with a nucleic acid sequence encoding for a sequence comprising the C-terminus of HAHB1, part thereof or a sequence comprising the HAHB1C-terminal consensus motif.

The invention also relates to a polypeptide encoded by this gene construct. The polypeptide is capable of conferring stress tolerance in a plant into which a gene expressing such polypeptide is introduced.

In one embodiment of this aspect of the invention, the C-terminal sequence of said polypeptide comprises or consists of SEQ ID NO. 8, 9 and/or 10. In another embodiment, the N-terminal sequence of said polypeptide comprises or consists of SEQ ID NO. 14 or a sequence with homology to the consensus sequence of SEQ ID NO. 14. Said homology is at least 80%, preferably at least 90%, more preferably at least 95% or 98%. In another embodiment, the polypeptide has the N-terminal sequence of an HD-Zip protein of subfamily I or the N-terminal consensus motif operatively associated with a sequence comprising or consisting of a sequence as defined in SEQ ID NO. 11 and/or SEQ ID NO. 12 or a sequence with homology to the C-terminal consensus motif as defined in SEQ ID NO. 11 and/or the C-terminal consensus motif as defined in SEQ ID NO. 12. Said homology is at least 80%, preferably at least 90%, more preferably at least 95% or 98%.

In another embodiment, the N-terminus of said polypeptide is the N-terminus of HAHB4.

The invention also relates to a method for conferring stress tolerance as defined herein in a plant which comprises introducing and expressing in a plant a chimeric gene construct above which encodes for a polypeptide or protein as defined above.

### EXAMPLES

Having generally described the invention disclosed herein, including methods by which those skilled in the art could make and use the invention, the following examples are provided to further extend this description, to enable those skilled in the art to practice this invention, including its best mode. However, the specifics of the examples which follow are not limiting. Rather, for an appreciation of the scope of the invention contemplated herein, reference should be had to the appended claims and the equivalents thereof.

### Experimental Procedures

Unless expressly stated otherwise, the following materials and methods were utilized in the non-limiting examples which follow:

### A. Constructs

35SCaMV:HAHB1: The cDNA was isolated from a library constructed in lambda gt10 as previously described (Chan & Gonzalez, 1992, and the sequence was provided to GenBank, see accession no. L22847, and SEQ ID. NO:2: herein for the nucleic acid sequence and SEQ ID. NO:5: herein for the translated protein sequence). This fragment was cloned in the EcoRI site of the pMTL22 vector and restricted with BamHI/SacI in order to clone it into the pBI 121 vector previously treated with the same enzymes. In this way, the cDNA expression is controlled by the 35S CaMV promoter.

HAHB1:GUS: The promoter region (SEQ ID. NO:1: herein) was isolated from a BAC genomic library using as probe the first intron (SEQ ID. NO:3: herein) of the gene previously isolated by PCR using two specific oligonucleotides. The approximately 125 kbp BAC insert was restricted with several enzymes, electrophoresed and hybridized in a Southern blot with the same probe. A positive 2000 bp HindIII fragment was subcloned in the pUC119 vector and sequenced. The insert presented a partial coding sequence plus the promoter region. The promoter region was amplified by PCR with the oligonucleotides PromHAHB1 (table a) and PR (table a) and cloned in the TOPO vector (Invitrogen). This plasmid was then restricted with XbaI and BamHI and finally cloned in the pBI101.3 vector. In this way the promoter of HAHB1 directs the expression of the GUS reporter gene. This construct was named HAHB1:GUS.

promHAHB1:HAHB1: HAHB1:GUS was restricted with BamHI/SacI and the HAHB1 cDNA was cloned into the vector, replacing the GUS gene. In this way the expression of HAHB1 is controlled by its own promoter.

These three constructs as well as pBI121 and pBI101.3 (used as controls) were used to transform DH5α Escherichia coli cells and then Agrobacterium tumefaciens.

Both, Arabidopsis glucanase and PR2 were cloned as follows: Arabidopsis (Col 0) genomic DNA was amplified with oligonucletotides glucanase CDS-R/glucanase CDS-F and PR2 CDS-R/PR2 CDS-F. The sequence of the oligonucleotides used for glucanase is shown in the sequence listing herein as SEQ ID NO. 23 and 24. The sequence of the oligonucleotides used for PR2 is shown in the sequence listing herein as SEQ ID NO. 21 and 22.

Once the DNA segments were obtained, they were cloned in the pBI121 vector previously restricted with BamH1/SacI replacing the GUS encoding gene. As with the other constructs, the cloning was performed in E. Coli cells and then Agrobacteria cells were transformed. Arabidopsis plants were then transformed following the floral dip method.

### B. Plant Material and Growth Conditions

Arabidopsis thaliana Heyhn. ecotype Columbia (Col-0) was purchased from Lehle Seeds (Tucson, Ariz.). Plants were grown directly in soil in a growth chamber at 22-24° C. under long-day photoperiods (16 h of illumination with a mixture of cool-white and GroLux fluorescent lamps) at an intensity of approximately 150 μE m⁻² s⁻² in 8 cm diameter×7 cm height pots during the periods indicated in the figures.

Helianthus annuus L. (sunflower cv. contiflor 15, from Zeneca) seeds were grown in soil in a culture room at 28° C. for variable periods of time depending on the purpose of the experiment as detailed in the figure legends.

### C. Abiotic Stress and Hormone Treatments

Sunflower seedlings (7-day-old) grown on wet paper were placed in liquid media containing the different hormones (in the concentrations indicated in the figure legends) and incubated for two hours. The plants were then frozen in liquid nitrogen; total RNA was extracted from each sample and analyzed by real time PCR (RT-PCR) as described below.

A similar procedure was followed with 21-day-old plants, but in this case, instead of whole seedlings, 1 cm diameter leaf disks (three disks for each treatment) were placed in the media containing the hormones. For seedling drought assays, the seedlings were placed for 30 minutes on a dry paper.

### D. Arabidopsis Transformation

Transformed Agrobacterium tumefaciens strain LBA4404 was used to obtain transgenic Arabidopsis plants by the floral dip procedure (Clough & Bent, 1998). Transformed plants were selected on the basis of kanamycin resistance and positive PCR which was carried out on genomic DNA with specific oligonucleotides. To assess HAHB1 expression, RT-PCR was performed on T2 transformants, as described below. Five positive independent lines for each construct (arising from at least two different transformation experiments) were used to select homozygous T3 and T4 in order to analyze phenotypes and the expression levels of HAHB1. Plants transformed with pBI101.3 were used as negative controls. For the other constructs, selection was similarly carried out and three to five independent lines chosen for the analysis.

### E. Transient Transformation of Sunflower Leaves

Sunflower leaves (in the R1 developmental stage; Schneiter & Miller, 1981) were infiltrated with 5 ml of Agrobacterium tumefaciens strain LBA4404 and then transformed with 35S:HAHB1 or 35S:GUS, used as control. After infiltration, plants were placed in a growth chamber for an additional 48 hr; 1 cm diameter disks (50 mg each) were excised from the infiltrated leaves and RNA was then extracted with Trizol (see below). For each gene transcript measurement, two disks coming from different plants were analyzed and the experiment was repeated at least twice. In order to test the efficiency of infiltration in these experiments, GUS reporter gene expression was measured by histochemical assays as previously described (Dezar et al., 2005b).

### F. Water Stress Treatments

Early water stress treatment in soil was carried out as follows: sixteen 8×7 cm pots, each with 120 g soil and 4 seeds, water-saturated, were placed in a 35 cm plastic square tray and cultured as described above except that further water was not added until severe damage was observed.

Water stress treatment was done on mature 4-week-old plants grown in the same culture conditions. At this age, no water was added again until stress became evident (approximately an additional 17 days). In both cases, photographs were taken two days after watering.

### G. Chilling Assays

Arabidopsis plants were grown in the same temperature and photoperiod as for water stress treatments during 14 days (for vegetative stage) or 25 days (reproductive stage). Then, they were placed in a special culture chamber with the same photoperiod and illumination conditions at 4° C. and maintained during the periods indicated in the corresponding figures.

### H. Freezing Assays

Two different assay types were performed. The first used incubations of 6-8 hours at −8° C. of non-acclimated plants grown in normal conditions. After the freezing treatment the plants were placed again in normal conditions and all the parameters measured after 6 days recuperation.

The alternative treatment used incubation of 14-day-old plants over 7 or 14 days at 4° C. and only after this acclimating treatment, were they subjected to −8° C. over 6-8 hrs. After that, the plants were placed at 4° C. for 24 hrs and then in normal growth conditions for 6 days.

### I. Salt Stress Treatments

To 14-day-old plants grown in normal conditions 1.5 L of 50 mM NaCl was added to the whole tray. Seven days after that, an additional litre of 150 mM NaCl was used to water the plants and finally seven days later another litre of 200 mM NaCl was added. In this way the salt stress occurs during the reproductive stage. Salt stress during the vegetative stage was generated by adding increasing concentrations of NaCl from 50 to 200 mM for watering of 10-day-old plants every 4 days.

### J. Chlorophyll Quantification

Extracts from 100 mg of leaves were prepared after freezing with liquid nitrogen. To each sample, 1.5 ml of 80% acetone was added and the tubes placed in darkness for 30 min. During this incubation the sample solids were decanted and the absorbance at 645 and 663 nm measured in the supernatants with a spectrophotometer. Chlorophyll concentration was quantified according to Whatley et al., (1963).

### K. Membrane Stability

Membrane stability was determined by the ion leakage technique. Leaves of each genotype were washed with distilled water before treating them with salt stress or low temperatures according to the experiment. After the corresponding treatment, they were placed in 15 ml distilled water and agitated for 1 hr at 25° C. After that, the conductivity (C₁, proportionally inverse to the stability) was measured in the liquid surrounding media. The final conductance (C₂) was determined in the supernatants after 4 hrs at 65° C. and one additional hour at 25° C. with continuous agitation. The damage index (L) was calculated as the ratio C₁/C₂. The basis for this technique is that the damaged tissues lose electrolytes and these electrolytes spread into the surrounding media and increase the conductivity. Values greater than 0.5 indicate severe damage (Sukumaran & Weiser, 1972).

### L. Hormone and Stress Treatments to Transgenic Plants

Transgenic plants (21-days-old) from the promHAHB1:HAHB1genotype were placed in 200 μM SA, 200 μM ABA or 20 μM ACC over 2 hrs. After the incubations, they were frozen and total RNA was extracted from each sample. Cold and drought treatments were carried out over 8 and 2 hrs respectively by placing the plants in a cultured chamber at 4° C. or on dry paper. Plants transformed with the pBI101.3 plasmid were used as controls.

### M. RT-PCR Measurements

RNA for real-time RT-PCR was prepared with Trizol® reagent (Invitrogen™) according to the manufacturer's instructions. RNA (1 μg) was used for the RT-PCR reactions using M-MLV reverse transcriptase (Promega). Quantitative PCRs were carried out using a MJ-Cromos 4 apparatus in a 25 μl final volume containing 1 μl SyBr green (10×), 10 μmol of each primer, 3 mM MgCl₂, 5 μl of the RT reaction and 0.20 μl Platinum Taq (Invitrogen Inc.). Fluorescence was measured at 80-84° C. during 40 cycles. Sunflower RNA was also prepared with the Trizol (Invitrogen Inc.) technique. Specific oligonucleotides for each gene were designed using publicly available sequences (www.arabidopsis.org). The designed sequences are as specified in Table a.

### N. Apoplast Protein Extraction and Chromatographic Purification

Apoplastic protein extraction was carried out essentially as described by Mauch and Staehelin (1989). Plant leaves (7 g) grown over 25 days in normal conditions and then alternatively acclimated over 10 days at 4° C. or placed for 3 h at 8° C. as indicated in the figures legend and treated as follows. After the acclimation period, they were infiltrated under vacuum for 20 min. in a solution containing 5 mM EDTA, 10 mM ascorbic acid, 10 mM β-mercaptoethanol, 1 mM PMSF, 2 mM caproic acid and 2 mM benzamidine. After infiltration, the leaves were dried over paper and placed in a 20 ml syringe. The syringe with the dried leaves inside was placed in a 50 ml tube and centrifuged for 20 min at 830 g. After centrifugation, the apoplastic extracts are recovered from the bottom of the tubes when they are not contaminated with chlorophyll. In such cases (chlorophyll contamination) the extracts were discarded. Protein concentration was determined by the Bradford technique. After that, the extracts were concentrated through Millipore Centricon Ultracel YM-10 columns equilibrated with 50 mM NH₄HCO₃. Aliquots of each sample (1 mg protein each) were purified through Sephadex G-200 columns previously equilibrated with 50 mM NH₄HCO₃ and the elution was performed with the same buffer and followed spectrophotometrically at 280 nm.

The proteins were analyzed in 12% SDS-PAGE carried out according to the Laemmli technique and visualized with Coomassie Brilliant Blue R-250 (Sigma). An Amersham Biosciences LMW calibration kit was used as a molecular weight marker. This kit allows the visualization of the following bands: 97, 66, 45, 30, 20 and 14.4 kDa.

### O. Recrystallization of Ice

Recrystallization assays were carried out essentially as described by Griffith (2005). To the apoplastic extracts (10 μl) obtained as described above from previously acclimated plants, 10 μl 26% (w/w) sucrose was added. Each mix, corresponding to the different genotypes, was treated as follows: 3 minutes at −80° C., 10 minutes at −20° C., 15 minutes at −8° C., 30 seconds at 4° C. and finally 1 hour at −8° C. A control was performed using buffer (10 μl) instead of apoplast extract. The samples were observed and photographed with a NIKON optical microscope.

### Example 1

### HARB1 Expression Pattern

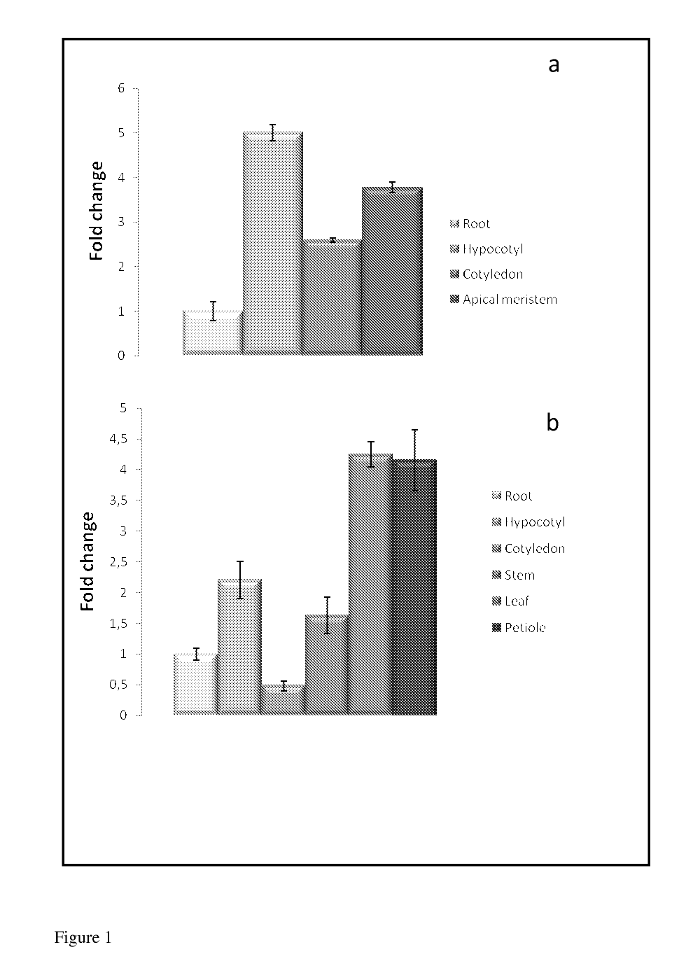

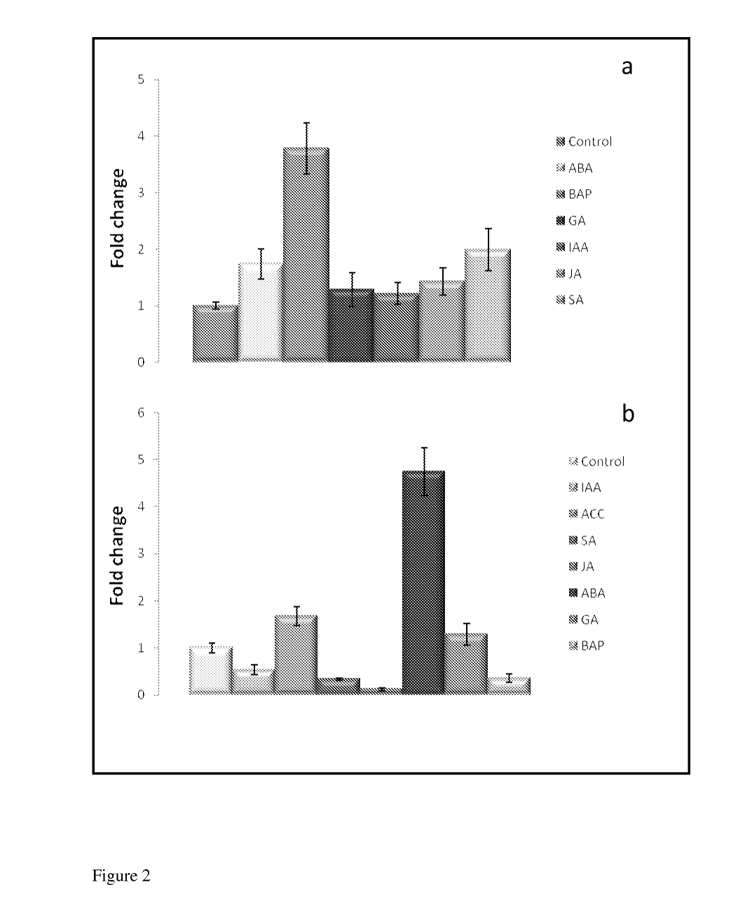

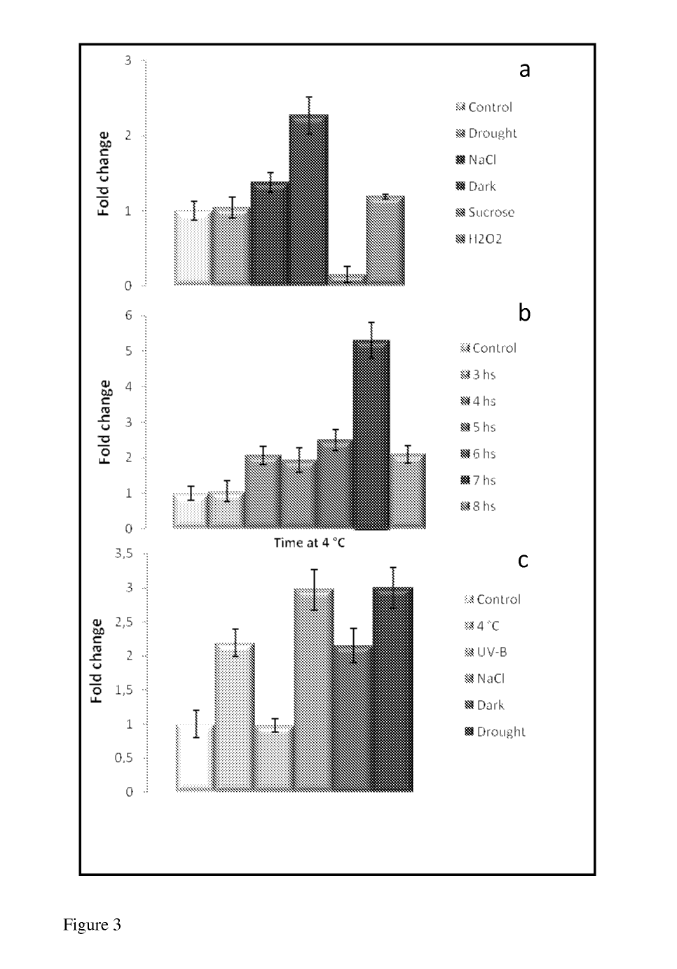

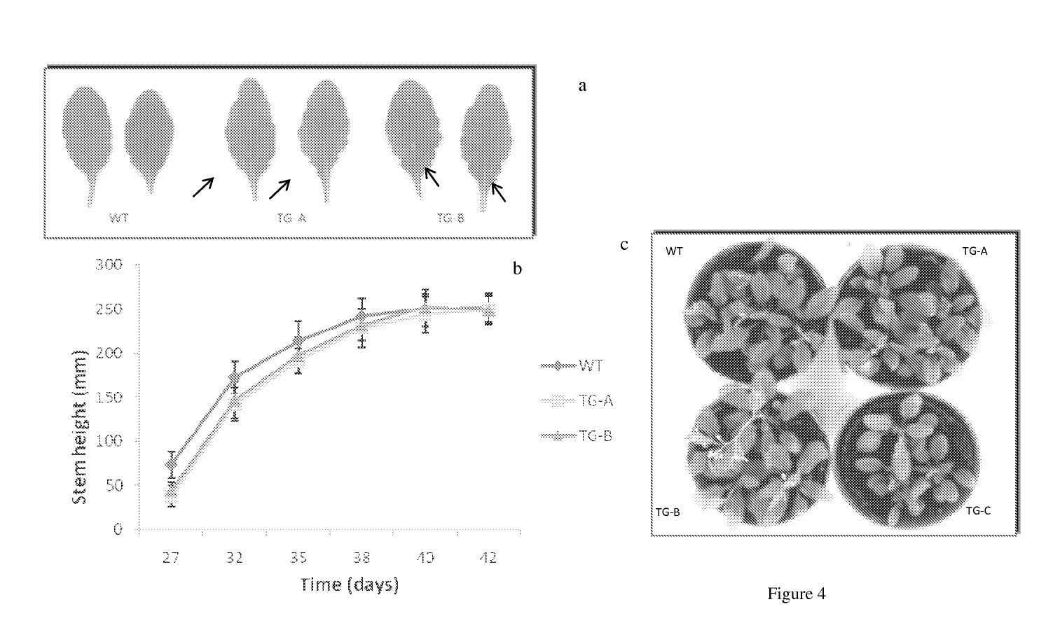

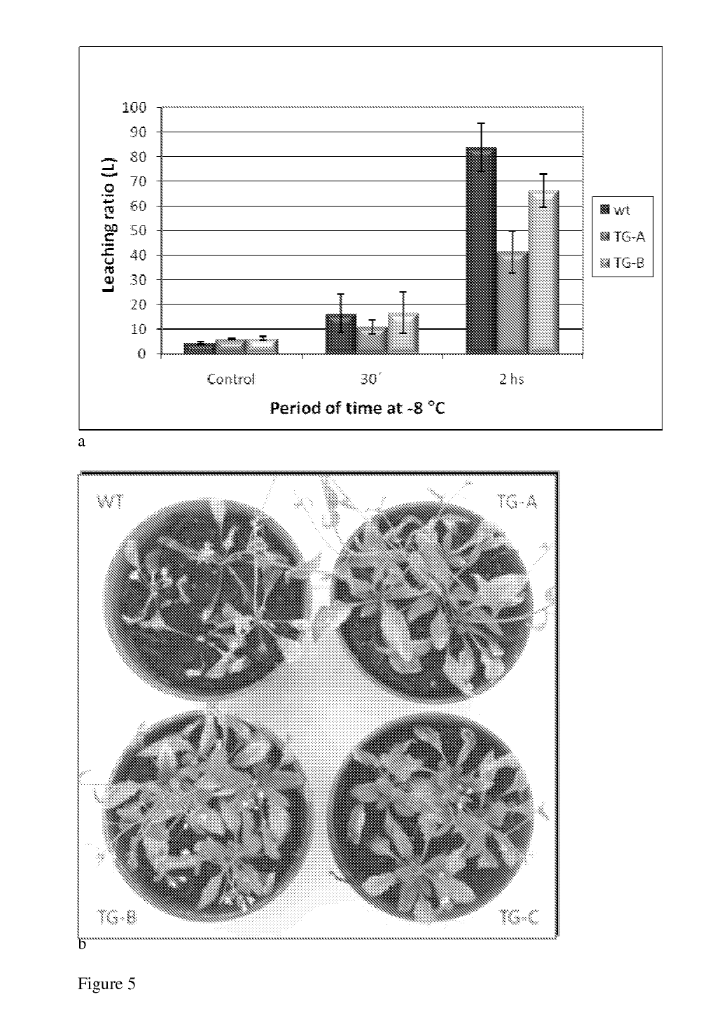

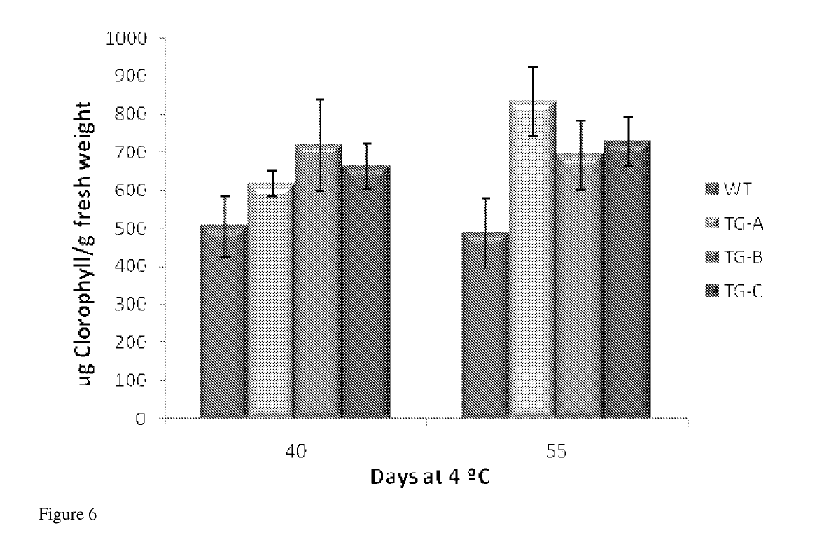

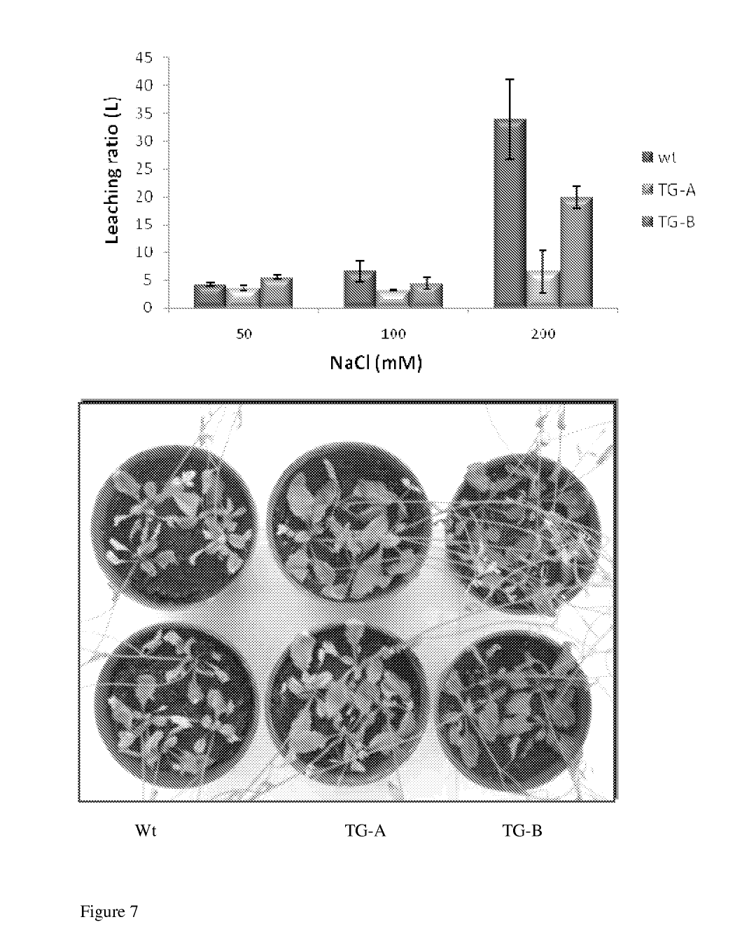

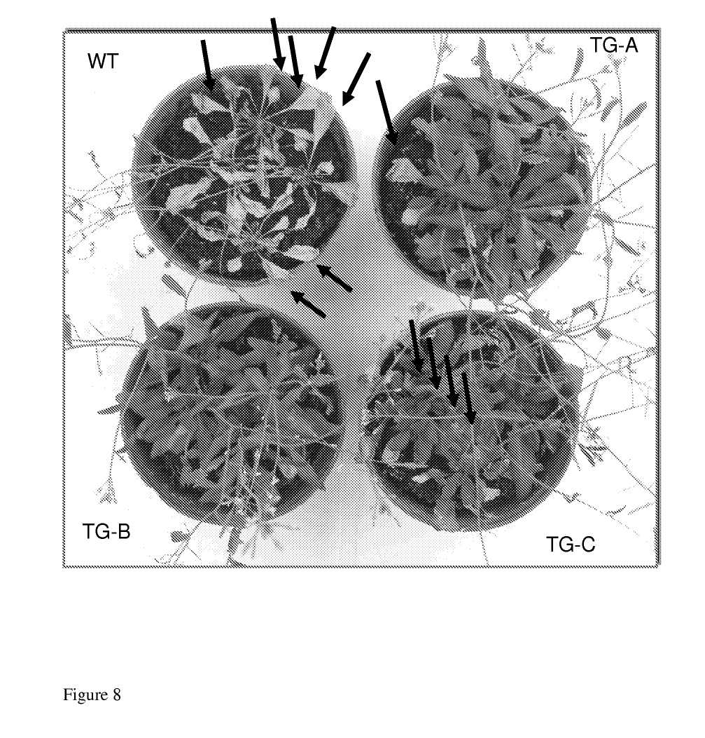

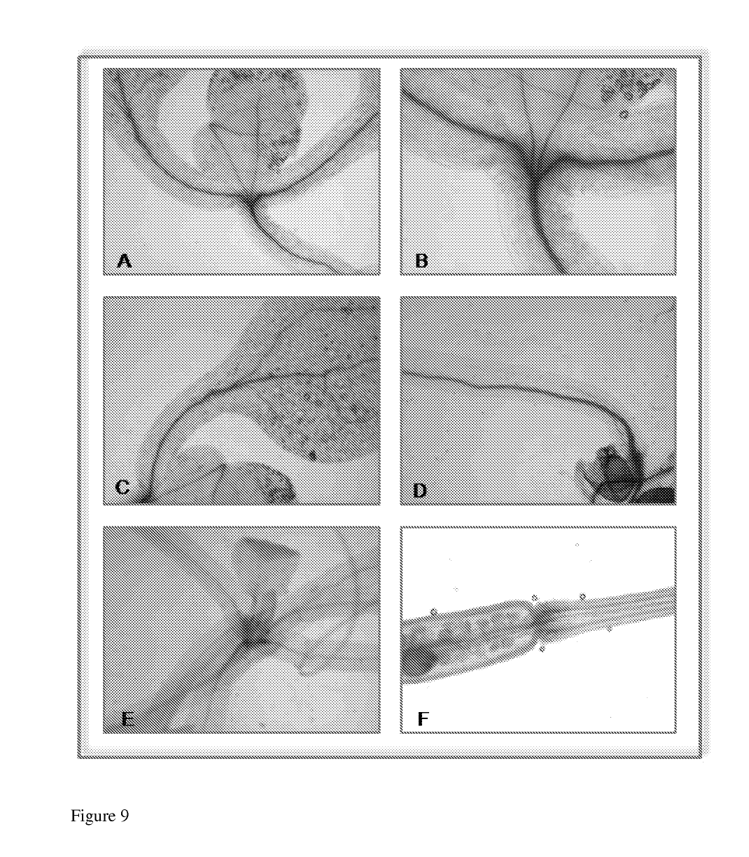

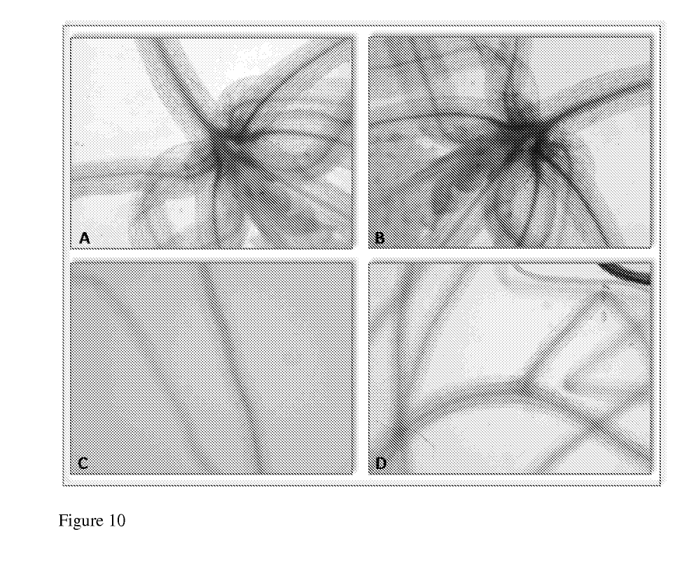

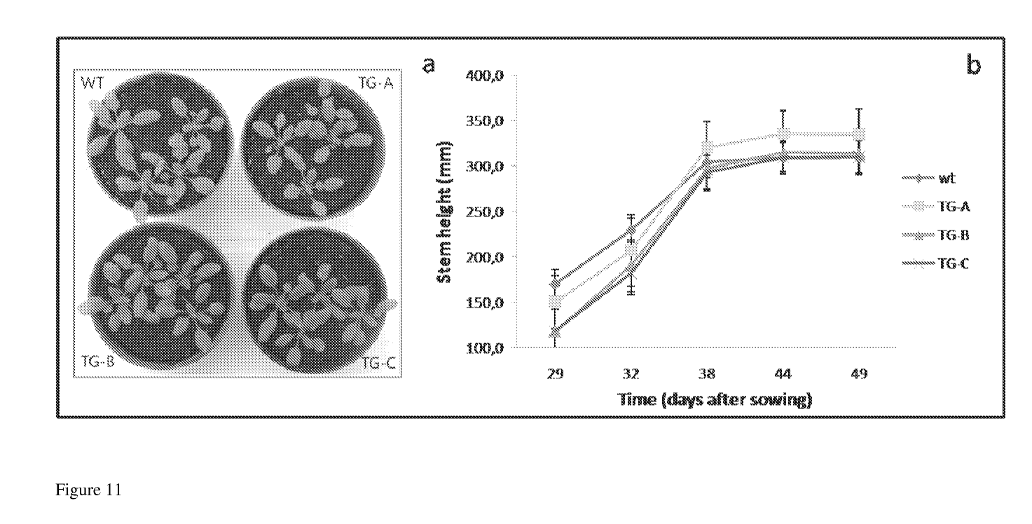

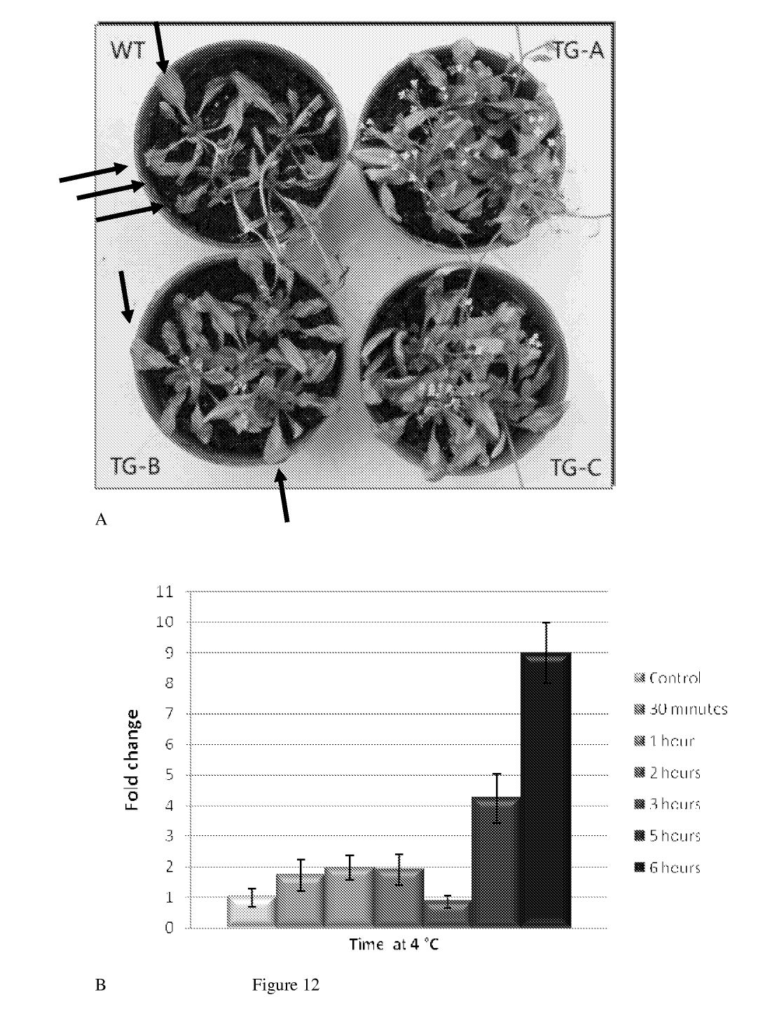

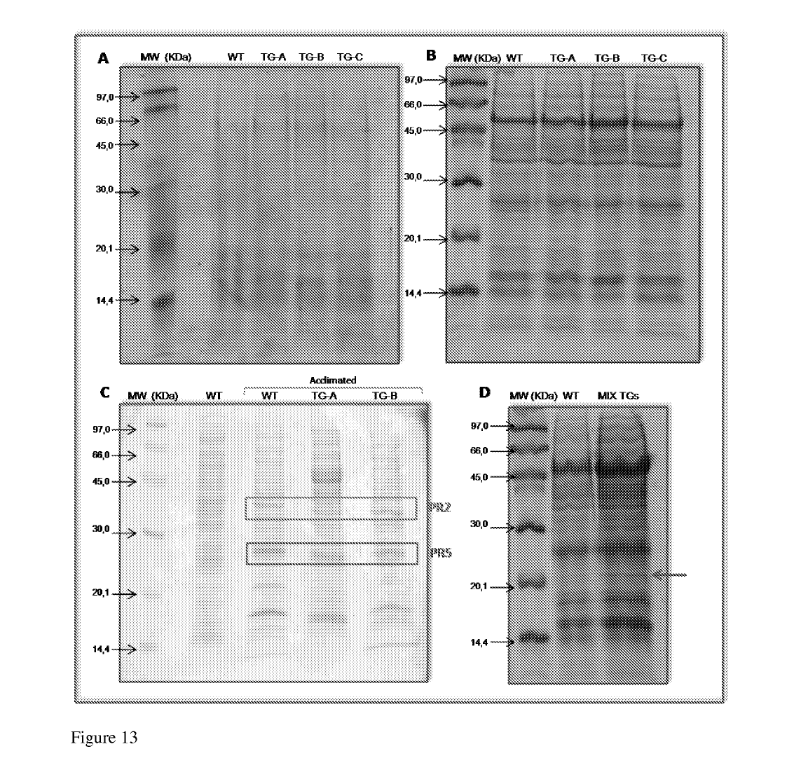

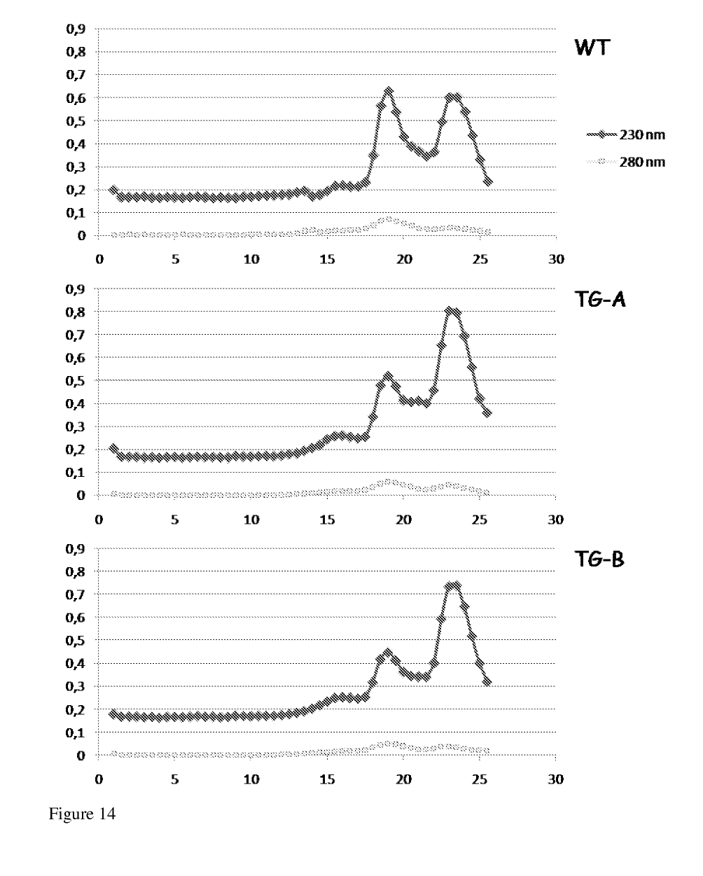

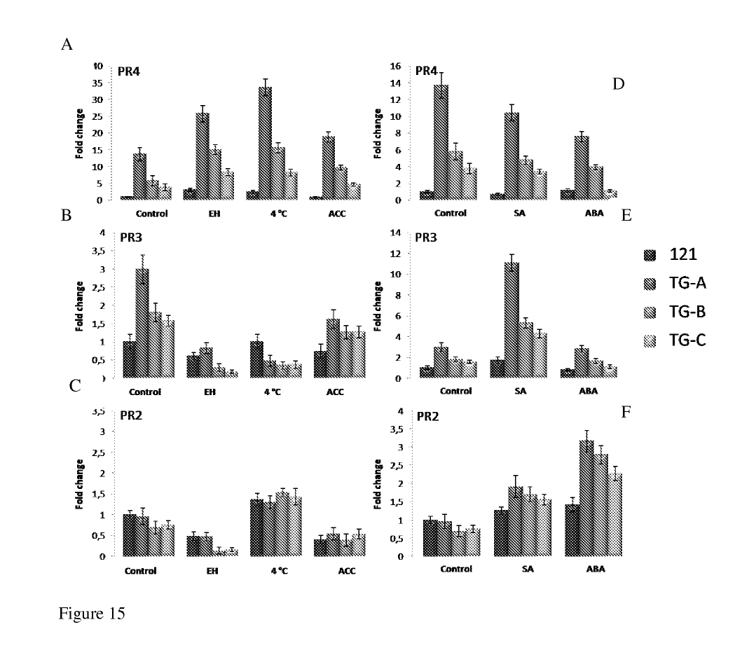

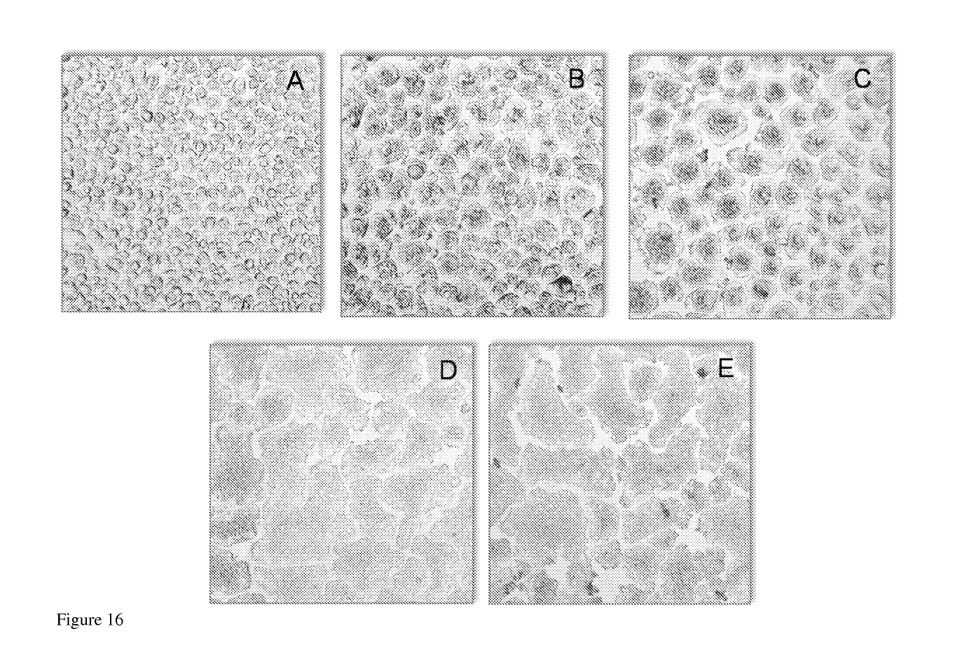

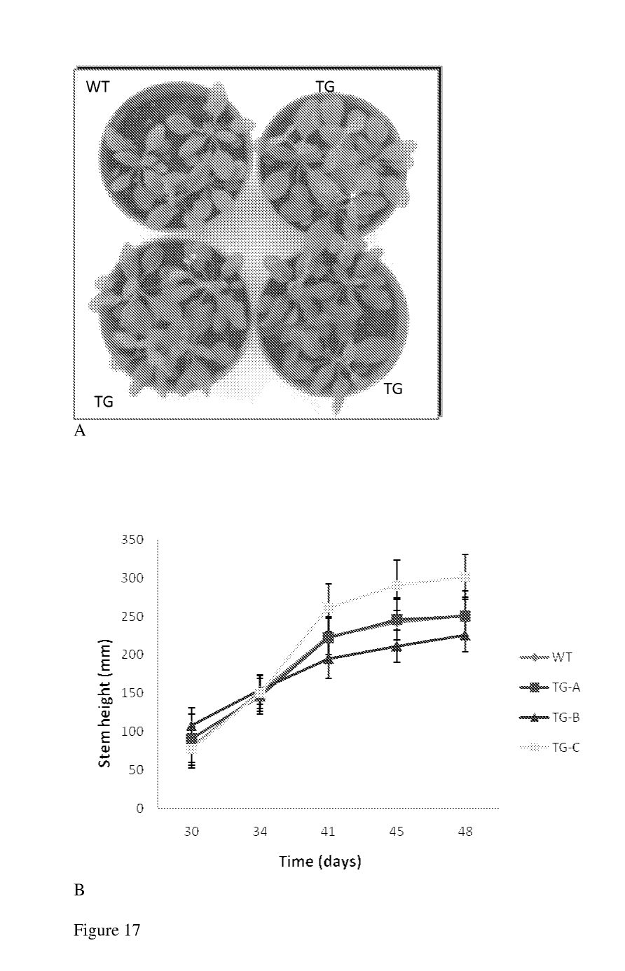

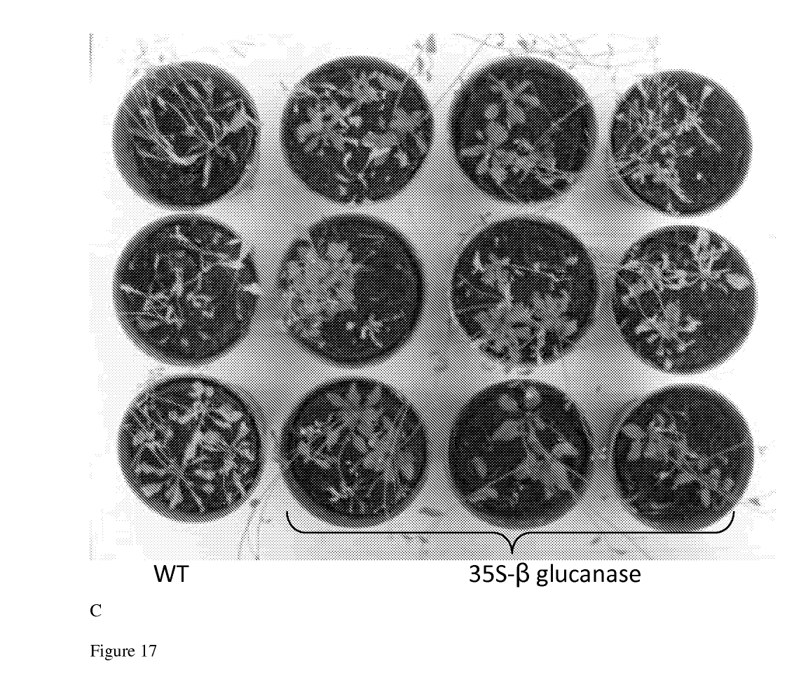

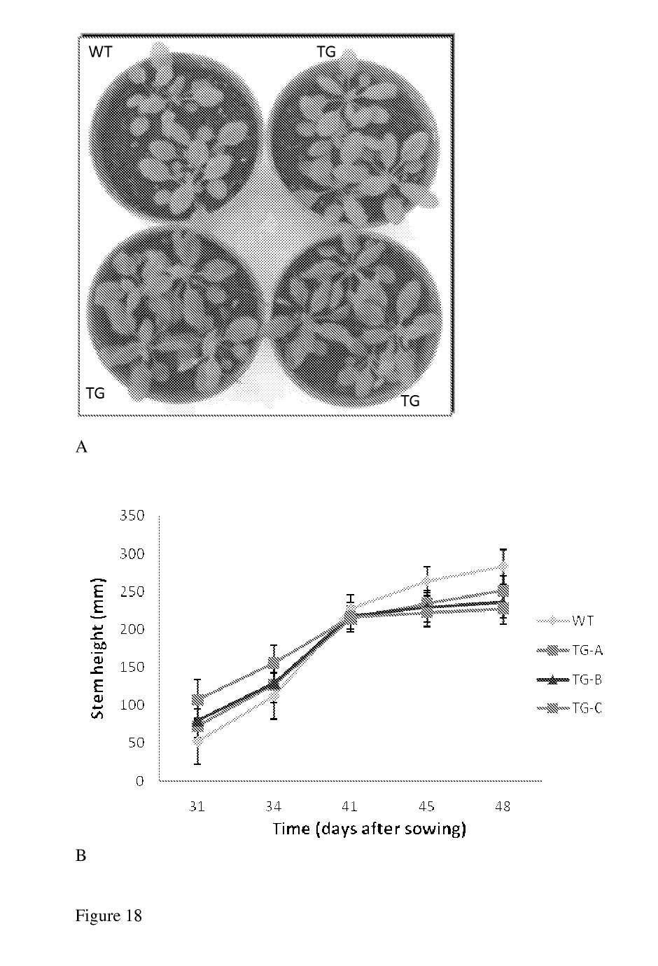

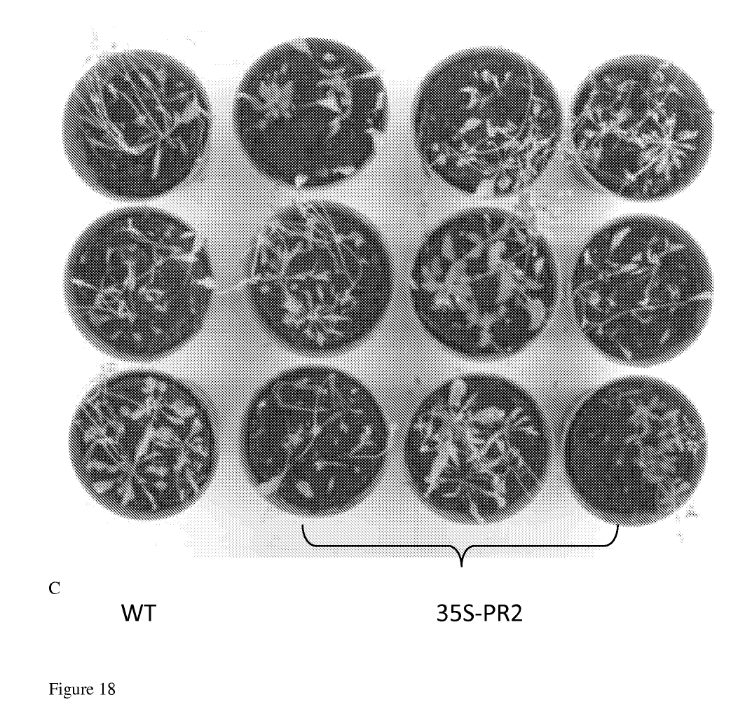

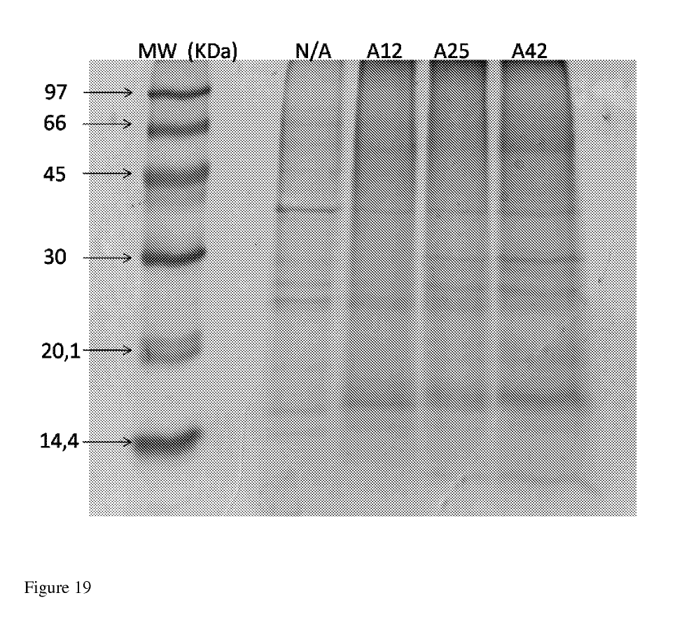

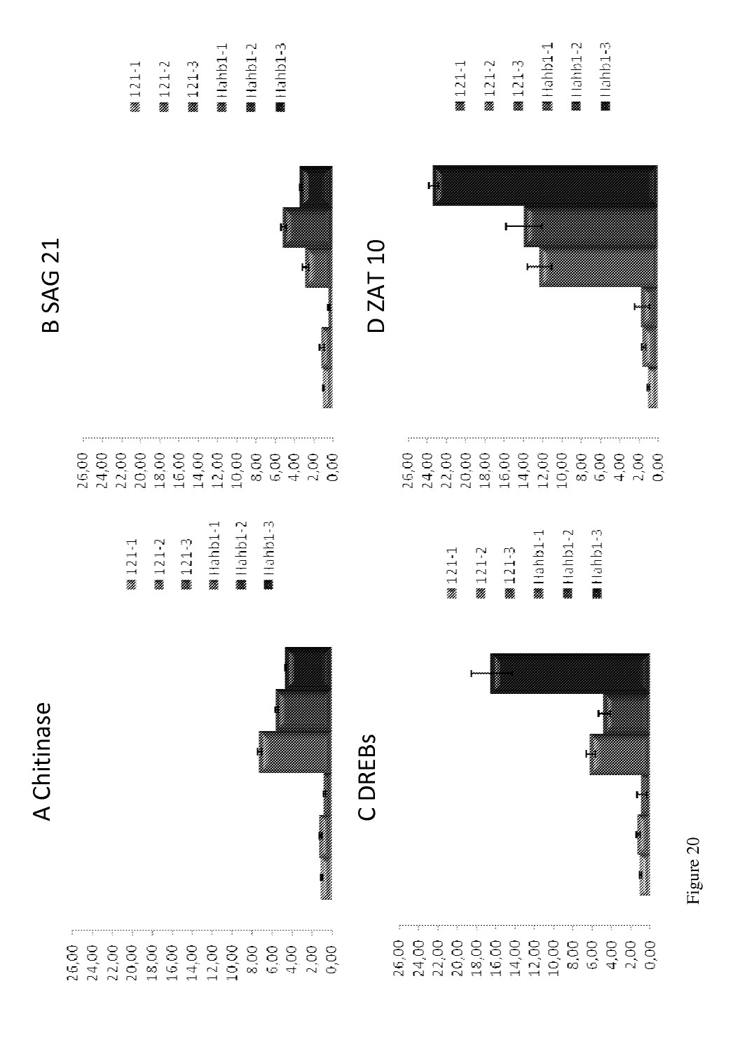

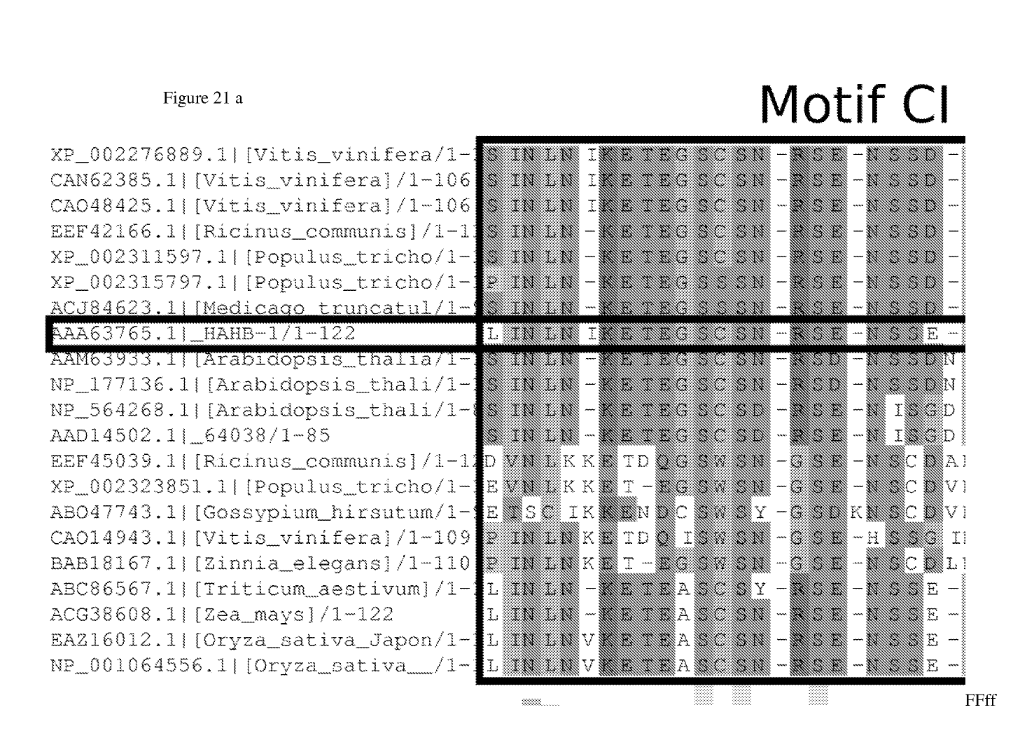

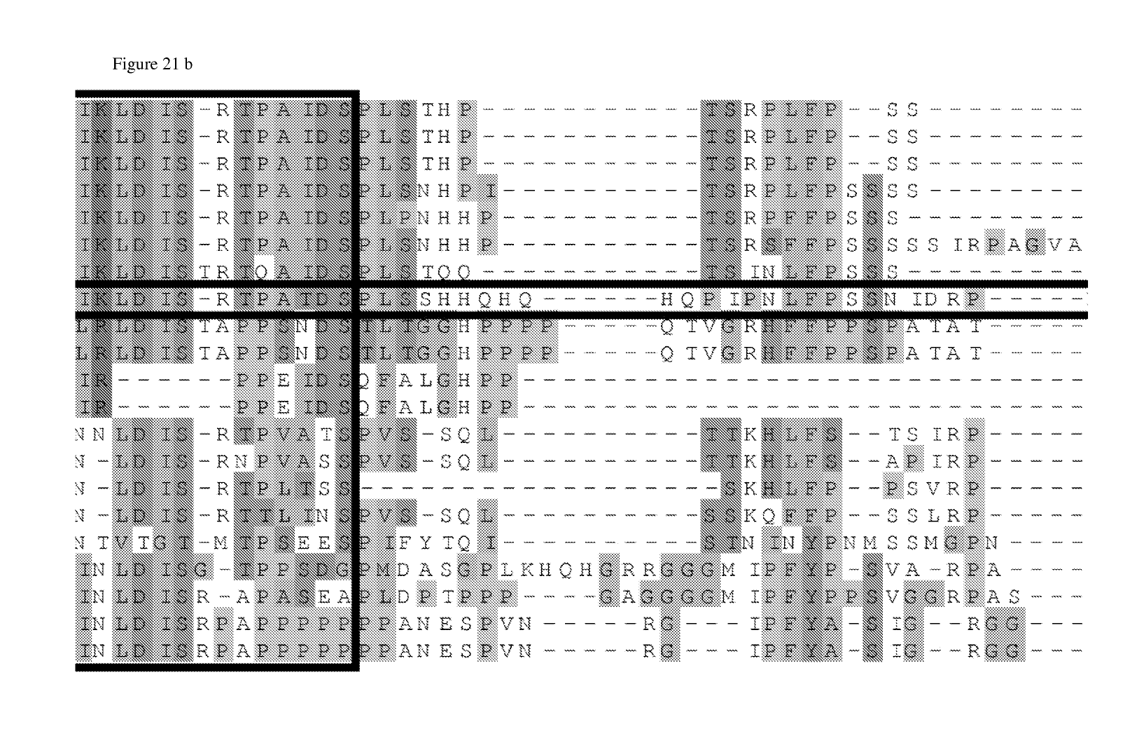

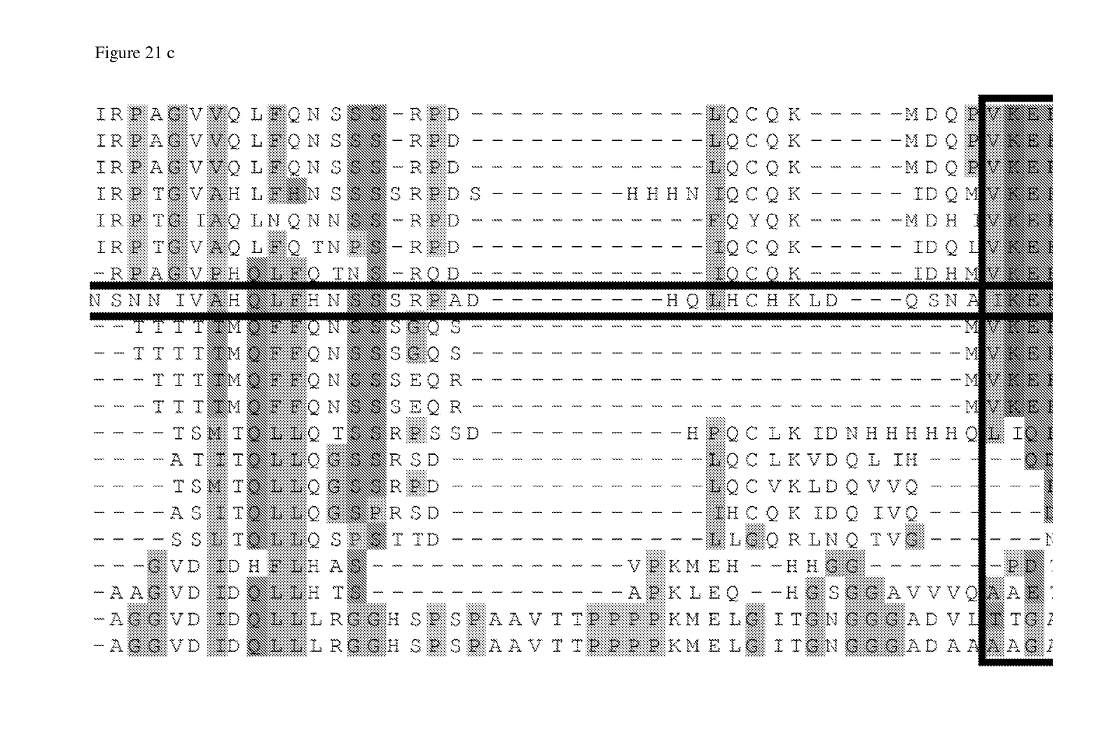

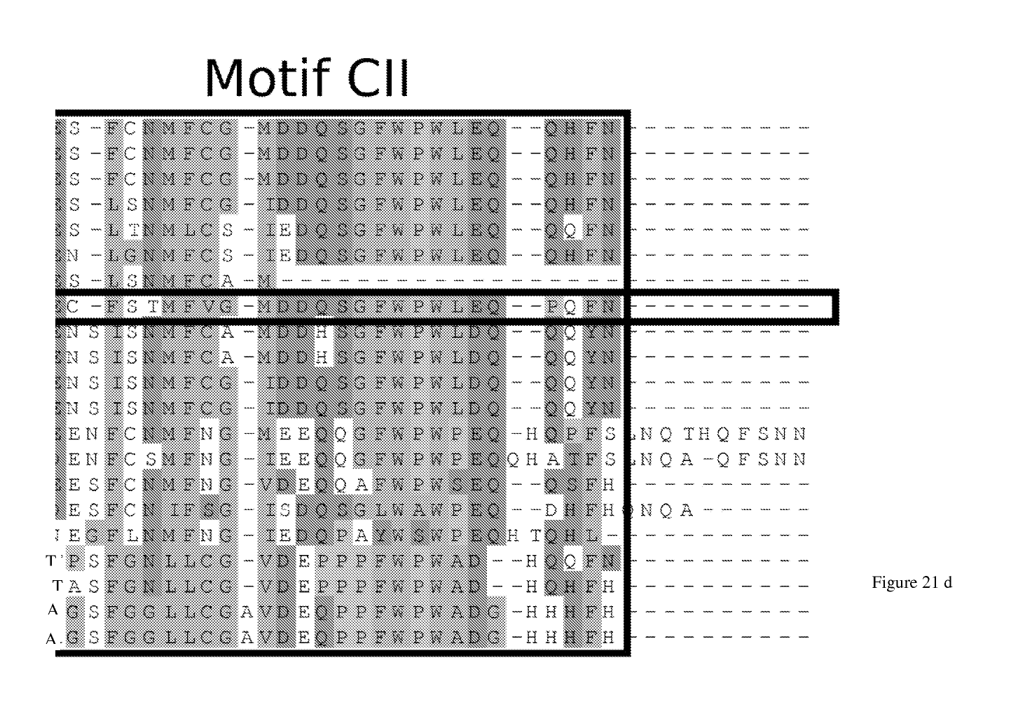

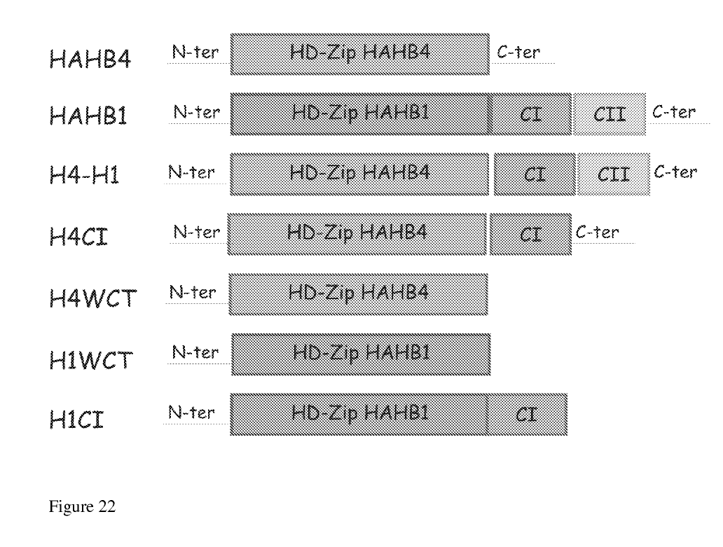

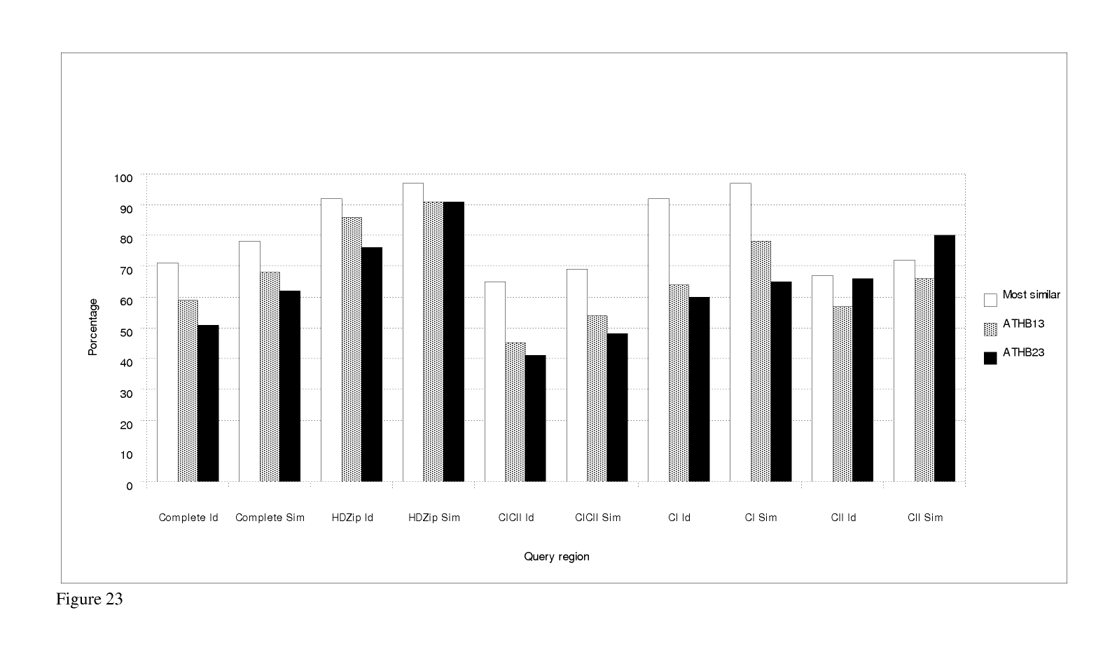

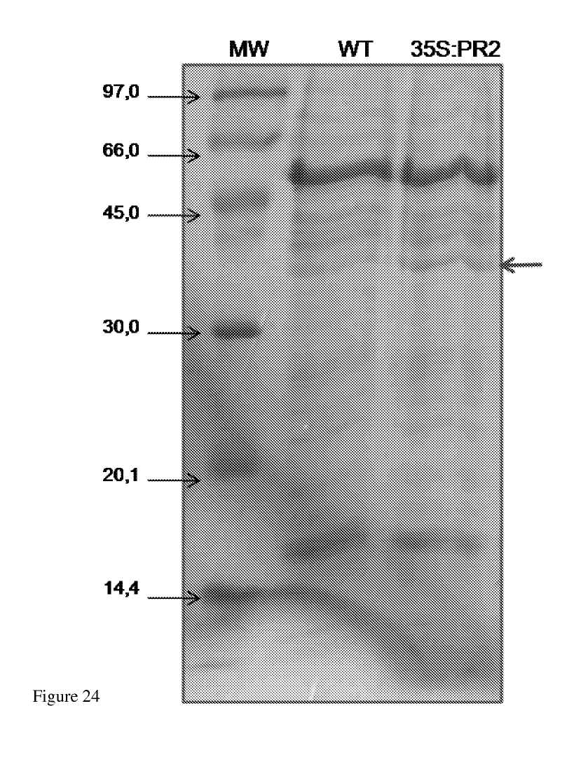

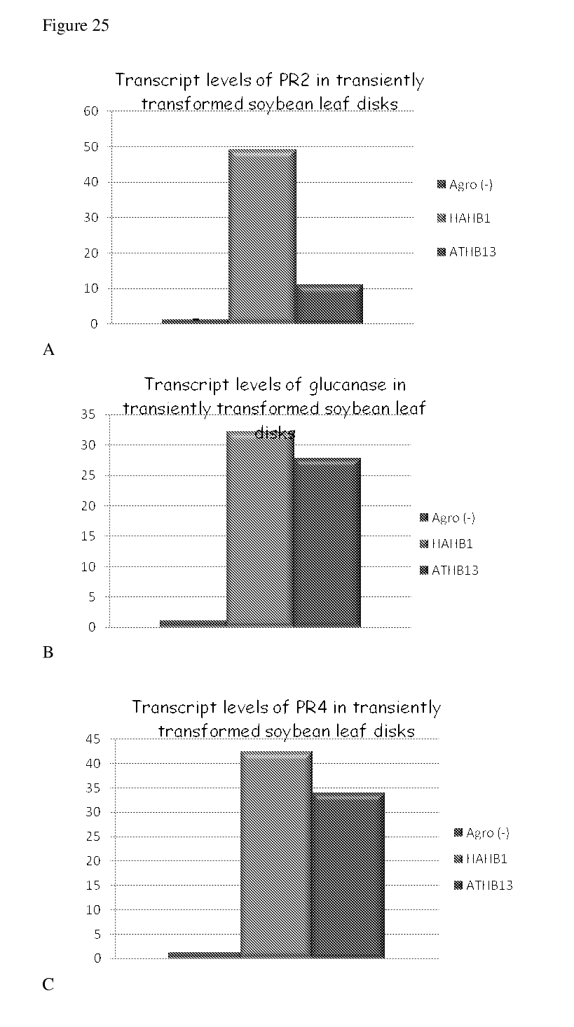

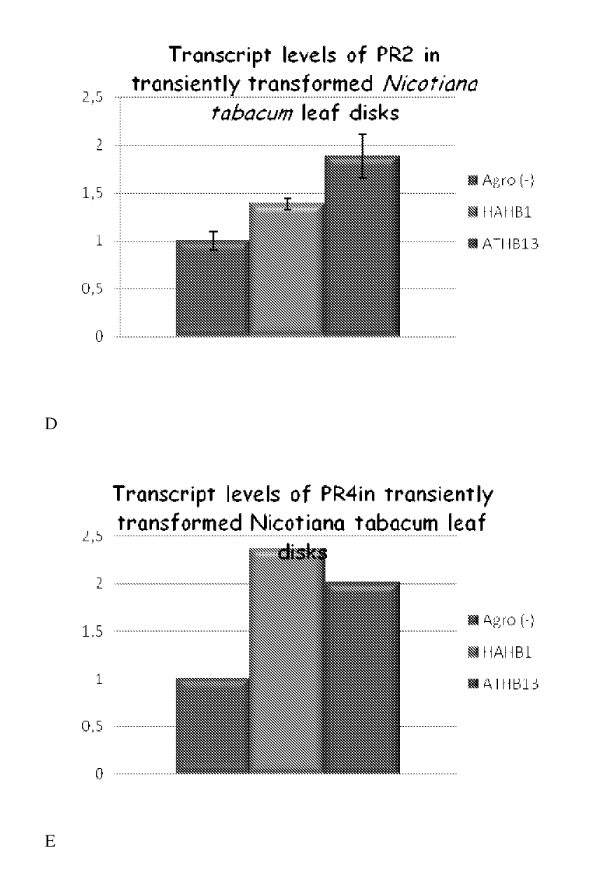

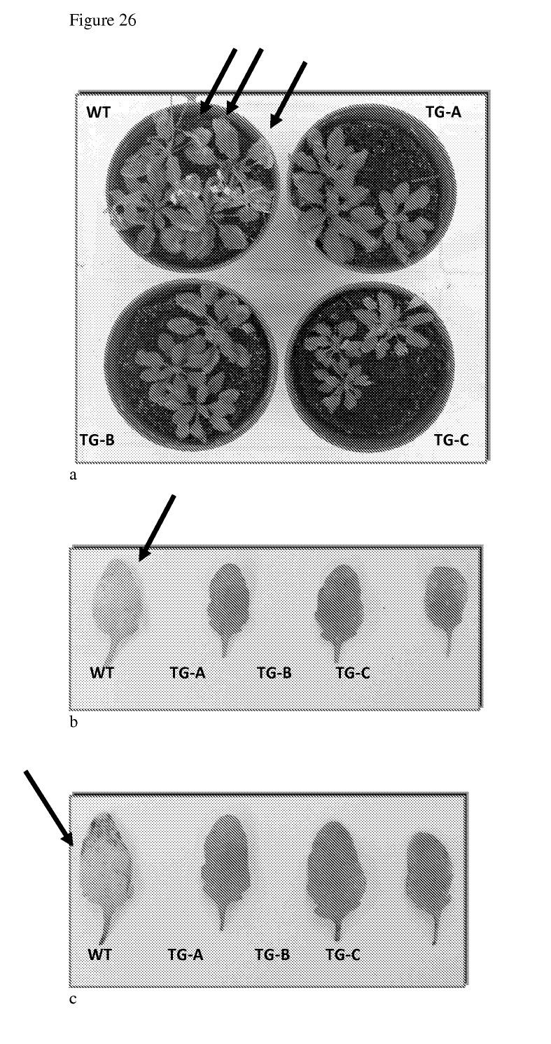

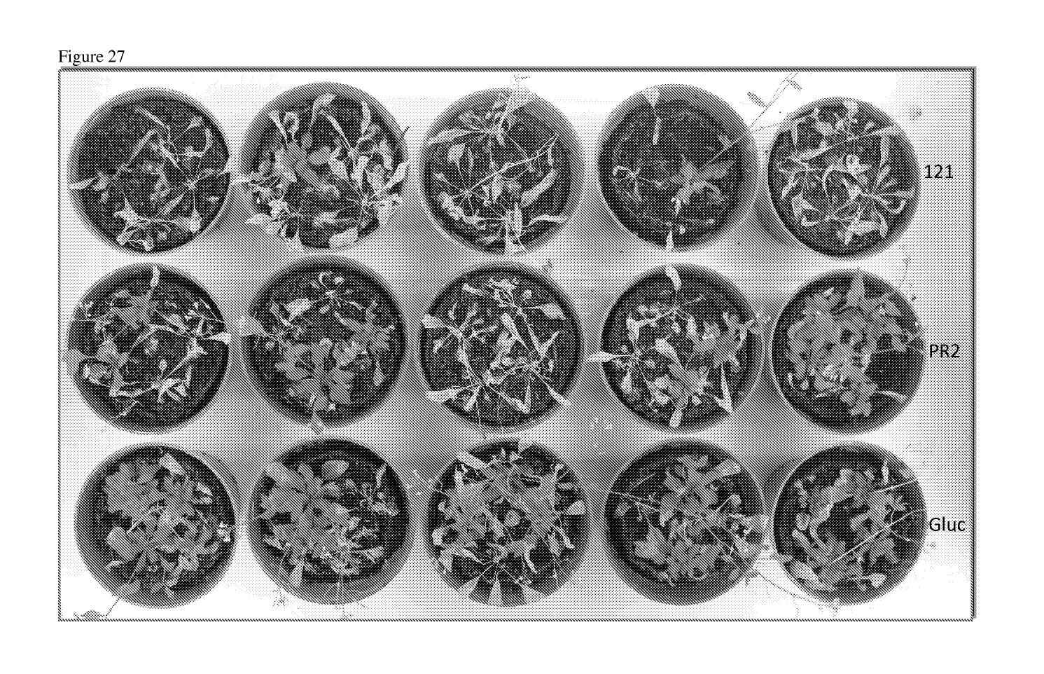

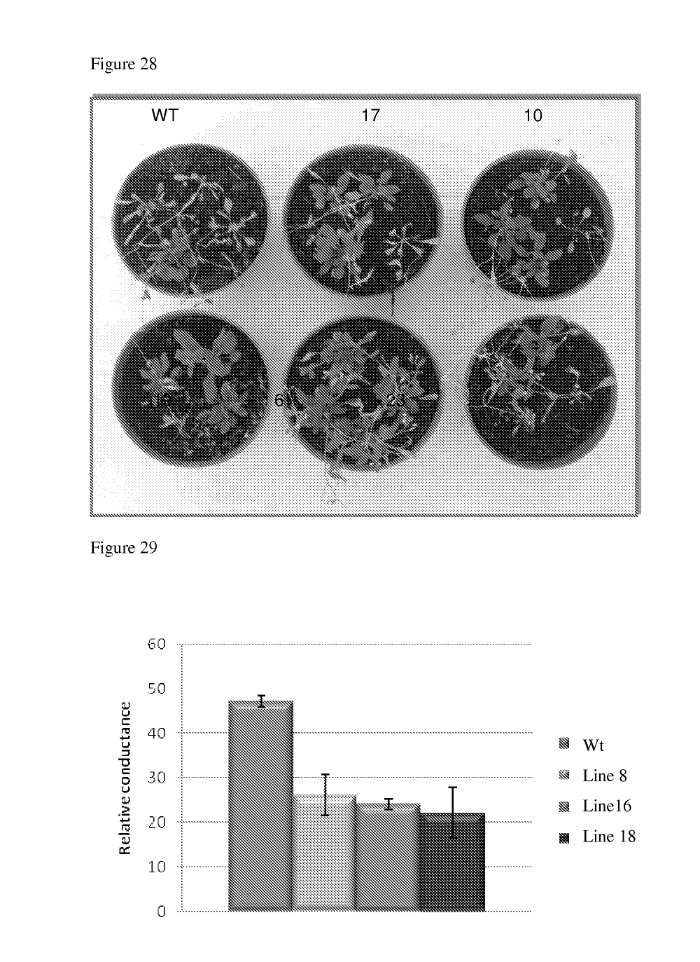

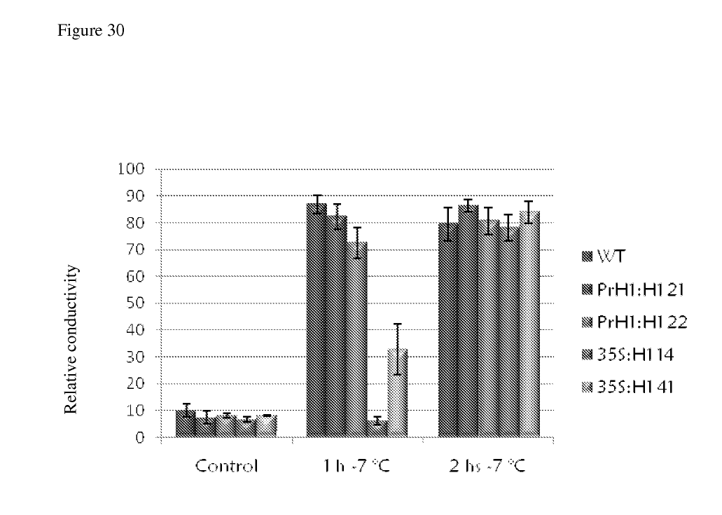

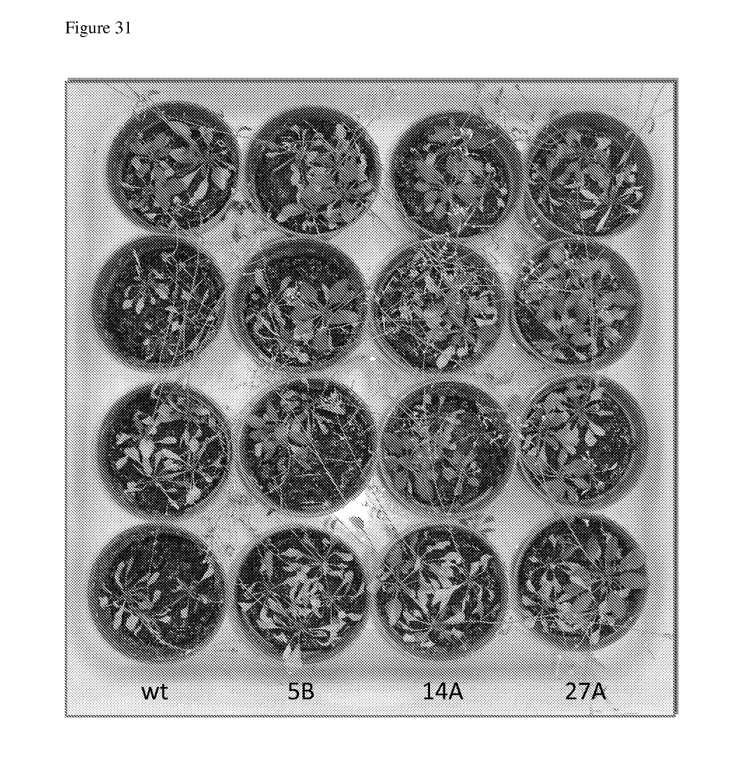

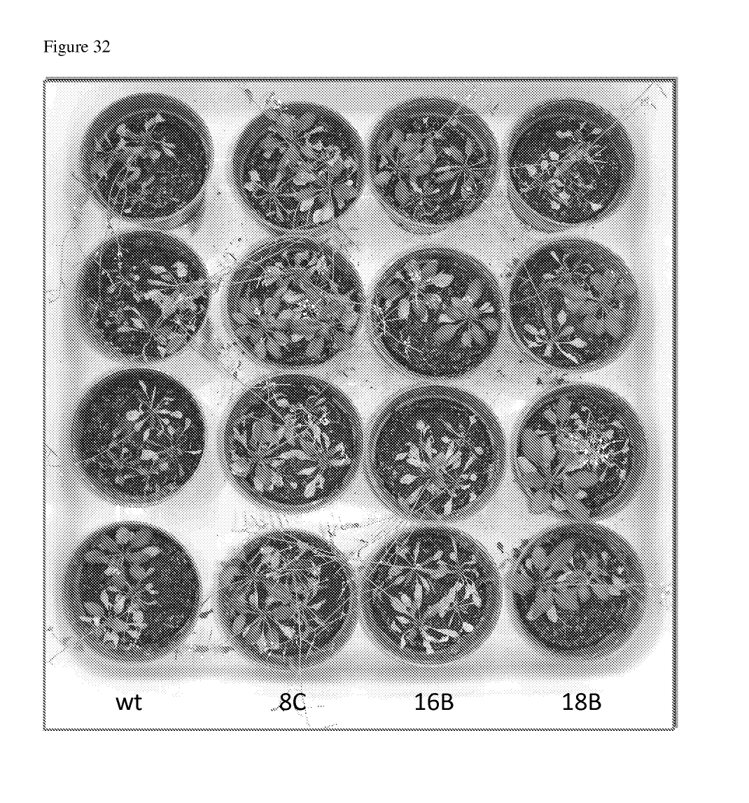

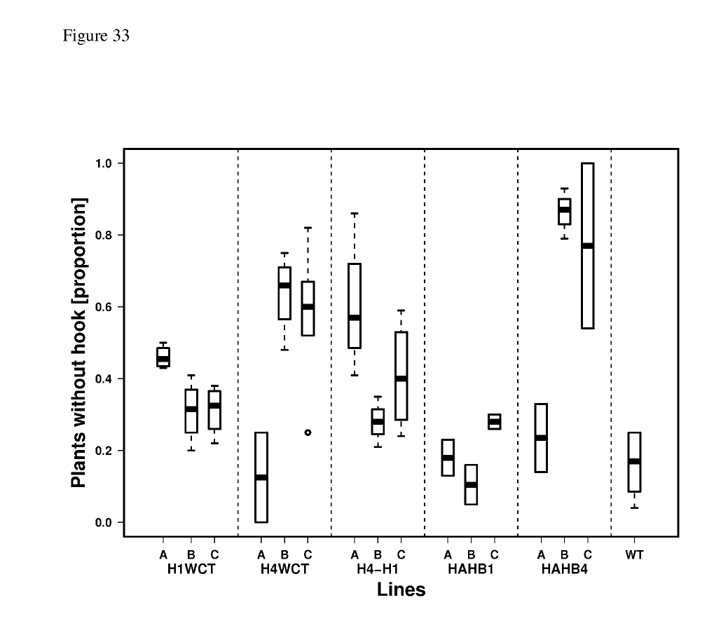

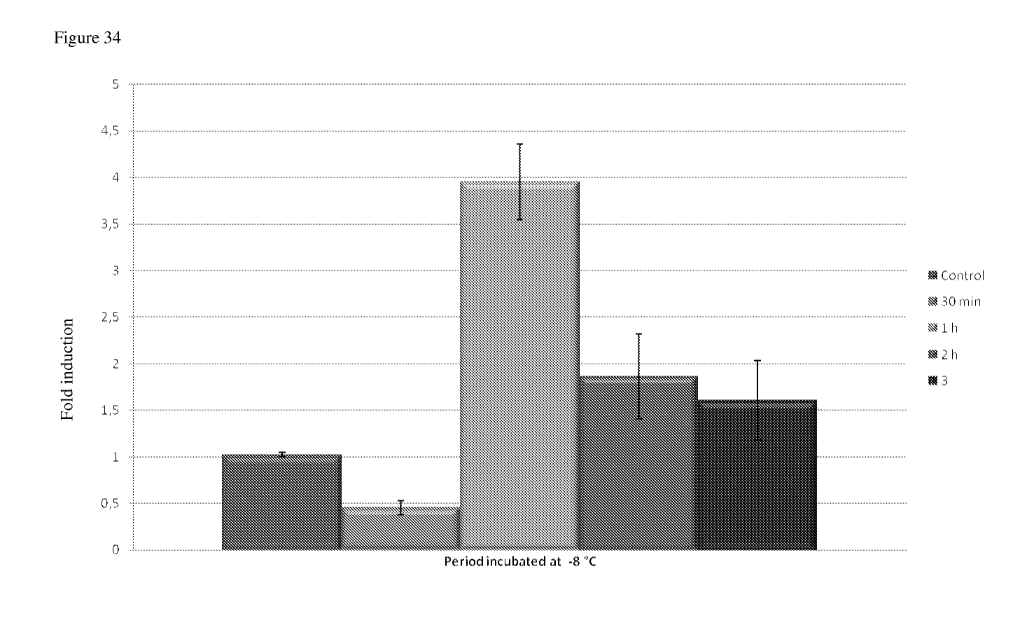

HAHB1 cDNA was isolated from a sunflower stem library (Chan & Gonzalez, 1994) but its function was not characterized. This cDNA encodes a 365 amino acid protein and seems to be a non divergent member of the HD-Zip I subfamily. In order to characterize expression of this gene, total RNAs were isolated from 7-day-old organs and these were analyzed by qRT-PCR as described in the Experimental section. FIG. 1a shows that at this stage of development the TF is mainly expressed in hypocotyls and apical meristems while the expression in cotyledons and roots is lower but detectable. This expression pattern changes in 21-day-old plants (FIG. 1b) showing primary expression in petioles and leaves and lower expression levels in roots, cotyledons, hypocotyls and stems.

### Example 2

### HAHB1 Expression is Up-Regulated by ABA, Cytokines and Abiotic Stress Effectors

We investigated the effect of some phytohormones in the expression of this gene in 7- and 21-day-old plants. The results, shown in FIGS. 2a and 2b, indicate that in seedlings BAP presents the higher effect on HAHB1 expression while ABA exhibits the major effect in leaves of more developed plants. Other phytohormones tested (IAA; ethylene (ACC), SA, JA and GA) either inhibitor, slightly induce or produce no effects at all on the expression of this TF. Sunflower seedlings (7-day-old) or developing plants (21-day-old) were subjected to different abiotic stress treatments in order to investigate the role of this gene in the response to environmental conditions.

Drought, salinity, oxidative stress caused by H₂O₂, and osmotic stress caused by sucrose all show low induction of expression of this gene in seedlings. Etiolated plants exhibit transcript levels two-fold greater than those observed in normal growth conditions (FIG. 3a). On the other hand, low temperatures (4° C.) induce the expression of HAHB1 more than five fold, indicating a significant up-regulation of this gene under this condition. A time course shows a maximal expression of HAHB1 after 7 hours of cold treatment (FIG. 3b). By contrast, in more developed plants (21-days-old), a different behaviour is exhibited. Several abiotic stress effectors such drought, NaCl high concentrations, darkness and cold temperatures induce the expression of the HAHB1 gene (FIG. 3c).

Together, the results indicate that HAHB1 is induced in mature leaves by various different abiotic stress conditions.

### Example 3

### Obtaining Arabidopsis Transgenic Plants Ectopically Expressing HAHB1

To confirm the function of HAHB1, we have used an ectopic expression approach. The coding region of HAHB1 was fused to the 35S promoter of Cauliflower mosaic virus, and the construct was used to transform Arabidopsis plants. Several homozygous lines were recovered. Five independent transgenic lines, named 35S:HAHB1-A, -B, -C, -D and -E, were selected for more detailed analysis.

FIG. 4a shows the difference in the leaf morphology between transgenic and wild type plants. Transformed plants show serrated borders and a differentiated shape in comparison with their non-transformed counterparts (FIGS. 4a and 4c). Regarding the development rate, transgenic plants present retarded stem elongation in early stages but reach a similar height at the end of the life cycle (FIG. 4b). The maximal difference in stem height between genotypes was observed when the plants pass from the vegetative to the reproductive stage. The number of rosette leaves and siliques as well as the seed productivity did not show significant differences between genotypes.

### Example 4

### Transgenic Plants Expressing HAHB1 are More Tolerant to Low Temperatures than their Wild Type Counterparts

In view of the known role of some HD-Zip proteins in abiotic stress responses, and our finding that HAHB1 is up-regulated at the transcriptional level by low temperatures, we investigated the behavior of transgenic plants bearing the construct 35S:HAHB1 when they are subjected to chilling or freezing temperatures at different developmental stages.

In order to test the behavior under freezing temperatures, the plants in vegetative stage were placed at −8° C. for 7 hours and then in a culture chamber in normal conditions to allow them to recover. FIG. 5 shows the results obtained. Between 57 and 85% (depending on the line) of transgenic plants expressing HAHB1 survived the treatment as compared with only 20% of the non-transformed plants. The severe stress affected the leaves of all of the genotypes, but the transgenic plants became healthy after a few days of recovery while the WT plants did not.

As mentioned above, low temperatures cause inhibition of photosynthesis and respiration as well as a repression in protein biosynthesis and induction of protein degradation. Carbohydrate transport is inhibited as well. All these effects share, as a common feature, the loss of membrane functionality.

In order to determine the membrane health after freezing treatment, we used the ion leakage technique (see Experimental Procedures) that allows quantification of the damage caused. Electrolyte release to the medium is an indicator of the severity of membrane damage and the conductivity in the surrounding solution provides a quantitative measurement of this damage. After centrifugation the apoplastic extracts are recovered from the bottom of the tubes unless they are contaminated with chlorophyll. In such cases (chlorophyll contamination), the extracts were discarded. Plants subjected for a range of different times to freezing temperature (−8° C.) were treated as described in the Experimental Procedures section above, and the conductivity measured. FIG. 5a shows that, after two hours of incubation at −8° C. the stability of WT plant membranes is lower than that of transgenic plants. At least in part, this explains the differences observed between genotypes in their survival under these conditions.

Regarding the composition of membrane lipids, no differences were detected in unsaturated and saturated fatty acid relationship between genotypes in plants subjected to chilling temperatures (data not shown) indicating that the observed freezing tolerance is not a consequence of a change in membrane lipids. Chilling also causes inhibition of photosynthesis. Chlorophyll concentration was measured in plants subjected to these conditions, both in vegetative and reproductive stages. FIG. 6 shows that chlorophyll content remained stable in transgenic plants in both stages while non-transformed plants progressively lost this pigment content indicating that transgenic plants better tolerate the adverse temperatures. It is important to note that in prolonged chilling conditions Arabidopsis plants almost stop their development and it becomes impossible to count survivors versus dead plants as a parameter of tolerance.

### Example 5

### Transgenic Plants Expressing HAHB1 are More Tolerant to Drought and Salt Stress than their Wild Type Counterparts

Transcript levels of HAHB1 increase when sunflower plants are watered with high salt concentrations as described above. In order to test if this gene participates in the plant response to this adverse condition, we irrigated the plants with increasing concentrations of NaCl and observed their behavior.

Membrane stability under these conditions was one of the chosen parameters in order to test plants behaviour. The obtained results are shown in FIG. 7a. The conductivity of the wild type genotype membranes notably increased during the treatment compared with that of the transformed one, indicating that membrane stability decreased accordingly. The difference between genotypes is more marked the greater the NaCl concentration.

The damage caused to plants is observable in a photograph two days after irrigating them with 400 mM NaCl (FIG. 7b).

In the same way, drought tolerance was also tested in transformed and non-transformed plants (FIG. 8). The treatment was severe, as described in the Experimental section. HAHB1 transgenic plants better tolerated the drought conditions and this tolerance correlated with the expression level of the HAHB1 transgene in the different independent lines.

### Example 6

### Isolation and Characterization of the HAHB1 Promoter Region

A 1060 bp segment corresponding to the promoter region of HAHB1 was isolated from a BACs genomic library using as probe the 5′-intron of the cDNA, previously isolated by PCR on genomic DNA with two oligonucleotides based on the coding sequence. Within this promoter region two boxes were identified as cold responsive elements by PLACE.

This segment was cloned so as to be operatively associated with the GUS reporter gene (i.e. expression of the GUS gene was under the transcriptional control of the HAHB1 promoter) and, in a separate construct, with the HAHB1 cDNA, as described in the Experimental Procedures section above. Both constructs were used to transform Arabidopsis plants and once homozygous lines from each were identified, they were analyzed as shown in FIGS. 9 and 10, which demonstrate the GUS expression pattern directed by the HAHB1 promoter. As can be seen, GUS is expressed in the vascular system of Arabidopsis seedlings and in the meristematic regions of adult plants (25-day-old and 40-day-old) in normal growth conditions.

### Example 7

### Phenotypic Characteristics of Plants Bearing the Construct promHAHB1:HAHB1

Transgenic plants bearing the construct promHAHB1:HAHB1 were produced, selected and grown in normal conditions and observed. No significant differences in shape, form or color as compared to their wild type counterparts were detectable (i.e. these transgenic plants are morphologically indistinguishable from wild type counterparts), see FIG. 11a). Regarding the growing curve, measured as the stem height during the life cycle, no significant differences were detected, indicating that under the control of its own inducible promoter, the gene does not produce a developmental delay in the early stages of plant development, as was observed with the constitutive 35S:HAHB1 construct bearing plants (FIG. 11b).

### Example 8

### Plants Transformed with the Construct promHAHB1:HAHB1 are More Tolerant to Freezing Conditions

In order to test if transgenic plants expressing HAHB1 under the control of its own promoter tolerate freezing conditions better than wild type plants, the plants were subjected after acclimation of 10 days at 4° C. to 6 hours at −8° C. and then for 2 days at 4° C. and then in normal temperature, to allow them to recover over a period of 6 days. The % of survivors of the transgenic genotype varied from 67 to 86% while the average rate of survival of the non-transformed plants was 40% in this assay. The assay was performed with 16 plants of each genotype. FIG. 12 illustrates the results achieved in this type of experiment.

### Example 9

### HAHB1 Confers Cold Tolerance to Transgenic Plants Via the Induction of Genes Encoding Antifreeze Proteins

The results presented herein above indicate that HAHB1 confers tolerance to several abiotic stress factors. To elucidate the molecular and physiological mechanisms involved in this response, since this gene encodes a transcription factor and it could therefore be regulating different transduction signal pathways, we performed a microarray analysis comparing the transcriptome of transgenic and wild type plants using RNAs extracted from both genotypes.

A simple analysis of the data obtained allows visualization of the up-regulation of several genes previously associated with abiotic stress in the HAHB1-expressing transgenic plants. Among these genes, the most relevant are those related to cell wall synthesis, to the pathogen response genes (i.e. PR encoding genes) and a β-1,3 glucanase gene. Glucanases, chitinases and thaumatin-like proteins have been described as functioning like antifreeze proteins when plants are subjected to low temperatures. Freezing temperatures lead to the formation of growing ice crystals inside and outside the cell, and the crystals are responsible for mechanical damage (inner crystals) and dehydration (outer crystals) of plants, respectively, and can result in plant death. Antifreeze proteins (AFPs) have been found and characterized in organisms of several kingdoms, including in fish, some insects, land arthropods, bacteria, fungi and plants. They have been purified from the apoplast region of plants and they function by locating themselves on the forming ice crystals. In this way, ice crystals adopt a bi-pyramidal shape that is unable to grow. A second way in which these proteins perform an antifreeze function is by preventing recrystallization (the formation of large crystals from small ones). Additionally, Tomczak et al. (2003) demonstrated that antifreeze proteins are able to directly interact with membranes, thereby inhibiting leakage. Accordingly, and without wishing to be bound by mechanistic considerations, the increased membrane stability observed in HAHB1 transgenic plants could, at least in part, be explained by the action of these proteins.

Accordingly, we isolated the apoplast proteins with the aim of analyzing the potential differences between genotypes. We did not observe very great differences between transgenic and wild type plants grown in normal conditions (FIG. 13A) or acclimated during 16 hours (FIG. 13B) or days (FIG. 12C), in SDS-PAGE but we were able to identify at least five bands secreted to the cellular apoplast in Arabidopsis not that, to the best of our knowledge, have not been previously described (FIG. 13 C). These polypeptides were sequenced and three of them match the sequences of PR2, PR5 and an unknown protein respectively. Unfortunately, the two others did not yield clear sequences and they appear to be less concentrated in transgenic plants than in wild type ones. We can speculate that these unidentified bands may be ice-nucleating proteins, the expression of which is repressed in HAHB1 transgenic plants. Surprisingly, when the plants, both transgenic and wild type, were subjected to freezing conditions at −8° C., the protein pattern observed was clearly different between genotypes. Transgenic plants bearing the 35S:HAHB1 construct exhibit a higher concentration of apoplast proteins (25 μg protein/g tissue in WT extracts versus 50 μg protein/g tissue in transgenic plants) as well as an additional band with an approximate molecular weight of 23 kDa, indicating a faster response in front of the adverse condition compared with their non transformed counterparts (FIG. 13 D). Such faster response could be, at least in part, responsible for the tolerance observed in this genotype.

Chromatographic eluates were spectrophotometrically measured at 230 and 280 nm, since it is known that antifreeze activity in winter rye apoplast can be measured in eluates at 230 nm (Griffith et al., 1992; DeVries et al., 1986). As can be appreciated from FIG. 14, the profiles corresponding to the transgenic genotypes differ from those of the wild type profiles.

According to Griffith and Yaish (2004), low temperatures and drought trigger a transduction signal pathway dependent on ethylene, resulting in the expression of proteins with antifreeze activity, while, if the induction is by exposure instead to cold or drought, is produced by SA, ABA or psycrophylic pathogens like snow mold, the induced signal transduction pathway results in the expression of proteins with antipathogen activity. Taking these reports in the literature into account, we analyzed the transcript levels of three genes encoding putative antifreeze proteins from the PR family treated or untreated with SA, ABA, drought, low temperature or ethylene in transgenic plants bearing the construct promHAHB1:HAHB1 in comparison with non transformed plants.

FIG. 15 illustrates this experiment in which the transcript levels of the genes encoding Arabidopsis PR4 (At3g04720), chitinase (PR3, At3g12500) and PR2, were quantified under various stress or non-stressed conditions. As it can be appreciated from the figure, PR4 is expressed in transgenic plants in control conditions between 4 to 15 fold more than in wild type plants. After exposure to drought, low temperatures or ACC, these levels increased in all the genotypes, while when the plants were treated with SA or ABA, the basal levels slightly decreased (FIG. 15).

PR3, also called chitinase-B, is more highly expressed in the transgenic genotypes than in controls, and it is significantly induced in the presence of SA. When ABA is applied, no changes were observed as compared to control conditions, while the other treatments (drought, ACC and 4° C.) produce a decrease in the transcript levels of this gene. On the other hand, PR2 is not induced in transgenic plants compared with wild type plants under normal conditions, but is induced in the presence of ABA or SA or in low temperatures, while it is repressed by treatments with ACC or exposure to drought. Together, these results could indicate that this gene may have a double function, both in cold tolerance and in response to pathogens.

### Example 10

### Apoplast Antifreeze Proteins Present in Transgenic Plants Expressing HAHB1 are Responsible for the Observed Freezing Tolerance

Antifreeze mechanisms are not well studied either in Arabidopsis or in sunflower. In view of the results obtained herein indicating that transcript levels of some putative antifreeze proteins increased in transgenic plants expressing HAHB1 and that the apoplast protein pattern is different in acclimated plants compared to non-acclimated plants, we investigated the antifreeze activity of these proteins in transgenic and wild type plants. A recrystallization assay with apoplast proteins was performed following the technique described in the Experimental section. As can be appreciated from FIG. 16, transgenic acclimated plant apoplast proteins inhibit the arrangement of small ice crystals into larger ones while wild type extracts did not exhibit this inhibitory effect. Among the transgenic lines, line A seems to be the most effective in achieving this function.

On the other hand, some of the genes identified in the microarray as presenting a putative antifreeze activity were chosen in order to obtain transgenic plants and subsequently evaluate their behavior under freezing conditions. Two of these selected genes, encoding PR2 and a β-1,3-glucanase, were analyzed. The genes were isolated by PCR with specific oligonucleotides using Arabidopsis genomic DNA. They were cloned into pBI 121.3 and were used to transform Arabidopsis. Transgenic plants were selected by kanamycin resistance and by the insertion of the genes after being checked by PCR. Five independent F2 lines (non homozygous) were analyzed in normal growth conditions as well as when the plants were subjected to freezing. FIGS. 17 and 18 show that neither PR2 nor glucanase expressing plants exhibit a differential phenotype in normal growth conditions, while both genes seem to confer freezing tolerance to transgenic plants. However, while glucanase expressing plants exhibit a 50% survival rate under severe freezing conditions (as compared with only 8% survival for WT plants; FIG. 17), PR2 plants show a lower extent (17% vs. 8%, FIG. 18) indicating that it is probable that both genes play a role in the freezing tolerance conferred by HAHB1 but not in the same proportion. In addition to experiments with heterozygous lines, we also carried out experiments with homozygous lines.

In order to corroborate the hypothesis stating that the induced PR like proteins are responsible for the antifreeze activity presented by HAHB1 expressing plants, we obtained transgenic plants expressing each of these genes independently. The selected three genes, encoding PR2, PR4 and a β-1,3-glucanase were isolated by PCR with specific oligonucleotides using Arabidopsis genomic DNA and cloned into pBI 121.3. The constructs were used to transform Arabidopsis and the correct insertion of the genes was confirmed by PCR. Five independent homozygous lines were analyzed in normal growth conditions as well as when the plants were subjected to freezing temperatures. We show that that neither PR2 nor GLUC nor PR4 expressing plants exhibit a differential phenotype in normal growth conditions, but all these genes seem to confer freezing tolerance to transgenic plants in freezing conditions. However, while GLUC expressing plants exhibit a 62% survival rate under severe freezing conditions (as compared with only 14% survival for WT plants), PR2 plants show a lower extent (42% vs. 14%) indicating that it is probable that both genes participate in the freezing tolerance conferred by HAHB1 but not in the same proportion (see FIG. 32). The transgenic plants also showed increased drought resistance (see FIGS. 31 to 32). Membrane stability, measured as the conductivity of the treated extracts was higher in the transgenic genotypes than in controls, thus indicating that this physiological mechanism is at least in part, responsible for the conferred tolerance.

### Example 11

### Arabidopsis and Sunflower Exhibit Conserved Mechanisms

In view of both the microarray data and the freezing tolerance conferred by HAHB1, we wondered if similar events as those described for winter rye involving AFPs occur in these plants. With the aim of answering this question we isolated apoplast proteins from acclimated and non acclimated sunflower plants and analyzed them by SDS-PAGE. FIG. 19 shows a time course of the protein pattern as a function of the acclimation days. As can be seen, under normal conditions (time 0 of the acclimation experiment) some bands appear in the sunflower apoplast. This was a surprising result since no proteins were detected under the same conditions in Arabidopsis. During the acclimation process, new protein bands appear whereas some of the original ones diminished or disappeared. We did not confirm to which protein each band corresponds, and this is not trivial in light of the fact that the sunflower genome sequence is not yet available. However, the experiment indicates that a mechanism of induction of AFPs and repression of ice-nucleating proteins is occurring.

On the other hand, using known ESTs and gene sequences, we have been able to identify some sequences that could be homologous to those identified in the Arabidopsis microarray induced by HAHB1. According to this identification we designed specific oligonucleotides in order to quantify these genes in transiently transformed sunflower leaves (see Experimental section). Four genes encoding one putative chitinase (Accession N^{o} TC18434), two putative transcription factors homologous to ZAT10 and SAG21 from Arabidopsis (Accession N^{o} of the sunflower genes TC16546 and TC19654 respectively) and one DREB-like (Accession N^{o} TC23839) transcription factor were analyzed. All of these genes exhibited higher transcript levels in sunflower discs overexpressing HAHB1 than in controls. Since the Arabidopsis homologous genes were also induced in transgenic plants, this result indicates that there is a conserved mechanism of action in both species controlled by the HD-Zip transcription factor, HAHB1.

### Example 12

### Functions of the Protein Domains of Members of Subfamily HD-Zip I

We analysed the structure of the C and N-terminus of HD-Zip I family members using standard techniques and algorithms and found consensus motifs.

For example, we analyzed the sequence logo of N-terminus to find consensus sequences in the HD-Zip and LZ domains. The ids of the Arabidopsis thaliana sequences used for the alignment were: AT5G53980.1, AT3G01470.1, AT4G40060.1, AT2G22430.1, AT5G65310.1, AT1G69780.1, AT1G26960.1, AT5G15150.1, AT3G01220.1, AT4G36740.1, AT2G18550.1, AT5G66700.1, AT5G03790.1, AT3G61890.1, AT2G46680.1. Consensus sequences were thus identified.

We also analyzed the Sequence of C-terminus to find consensus sequences using standard techniques and bioinformatics. The ids of the proteins used to produce the sequence logo are: BAA05625.1, BAA05623.1, XP_{—}002276889.1, CAN62385.1, CA048425.1, EEF42166.1, XP_{—}002311597.1, XP_{—}002315797.1, AAT40488.1, AAT40518.2, AF011556_{—}1, ABL63116.1, AAD14502.1 (all previous by gene id), HAHB-1 (from sunflower) and ATHB13, ATHB23 (from Arabidopsis thaliana).

The alignment of the C-terminus from these 15 proteins (most homologous to HAHB1 C-terminus) belonging to different species plus HAHB1 was used to obtain 2 consensus sequences for the 2 best conserved regions, one at the beginning (N end), and the other located at the C end. In the whole carboxy terminus region, there are two conserved domains, one located 5′ and the other one in the 3′ of the region. The amino acids between these two motifs are not conserved.

We also constructed chimeric proteins fusing the HD-Zip domain of one protein with the carboxy-terminal domain of another. The constructs we produced are shown schematically in FIG. 22. With these constructs, we transformed Arabidopsis plants, obtained homozygous lines and analyzed the phenotypes, especially regarding characteristics conferred by wt proteins and HAHB1, as compared, for example, to the known characteristics and effects of HAHB4 (see Dezar et al, 2005a and b, Manavella et al 2006, Manavella et al 2008a, b and c. Cabello et al 2007; and WO2004/099365).

The plants transformed with the construct H4CI (comprising the HD-Zip of HAHB4 plus the CI of HAHB1) exhibit the leaf and inflorescence morphological phenotype conferred by HAHB4 (compact), while the plants transformed with H4CICII (comprising the HD-Zip of HAHB4 plus the whole carboxy terminus of HAHB1) exhibit the leaf morphological phenotype conferred by HAHB1 (serrated leaves and equal rosette leaves number).

Regarding ethylene sensitivity in etiolated seedlings, H1WCT (HAHB1 without its whole carboxy terminus) and H₁C1 (HAHB1 without the CII) plants behaved like HAHB4 plants (they did not present the triple response, Manavella et al., 2006). Ethylene sensitivity assay in adult plants showed that H4CII (comprising the HD-Zip of HAHB4 plus the CII of HAHB1) and HAHB4 plants behaved similarly (low senescence induction) while HAHB1, WT and H4-H1 (H4CICII) quickly entered in the senescence stage (see FIG. 33).

HAHB1 transformants, like Arabidopsis ATHB13 transformants, show rounded cotyledons when grown in 4% sucrose. In this kind of assay H4CI, HAHB4 and WT did not change their morphology while H4-H1 (H4CICII) and HAHB1 exhibited the described phenotype.

H4-H1 (H4CICII) transformed plants exhibit the same morphology as do HAHB1 plants. Both HAHB4 and HAHB1 appear more tolerant to drought than WT. HAHB4 plants are more tolerant than HAHB1 transgenics.

In chilling assays, the H4CI genotype seems to behave like the HAHB1 genotype (tolerant) using HAHB4 plants (non-tolerant) as internal controls.

These results confirm that the carboxy-terminus is responsible for the different functions exerted by HAHB1 as compared with those induced by HAHB4, which belongs to the same subfamily.

### Example 14

### Overexpression of ATHB13 in Arabidopsis

The cDNA was amplified using RNA isolated from 21-day-old plants by RT-PCR. PCR was carried out with two specifically designed oligonucleotides and cloned in the pBI 121.3 vector previously restricted with BamH1 and XbaI. Once a positive clone was identified, Agrobacteria cells were transformed with the construct and these Agrobacteria used to transform plants by the floral dip procedure. Transgenic plants were selected by kanamicyn resistance and then the insertion of the gene checked with two oligonucleotides, one matching the 35S (to differentiate the insertion from the endogenous gene) and one matching the cDNA. Once homozygous lines were identified, the transgenic plants were subjected to the same treatments as the HAHB1 plants with the appropriate controls.

### Example 15

### Sunflower, Soybean and Tobacco Leaf Discs Transformed with 35S:HAHB1 Over-Express Genes Putatively Related to the Cold Response

In view of both the microarray data and the freezing tolerance conferred by HAHB1, we wondered if similar events as those described for winter rye involving AFPs occur in other plant species. With the aim of answering this question, using known ESTs and genes sequences, we have been able to identify some sequences in sunflower, tobacco and soybean, homologous to those identified in the Arabidopsis microarray as induced by HAHB1. According to this identification we designed specific oligonucleotides in order to quantify these genes in transiently transformed sunflower, tobacco and soybean leaves both with 35S:HAHB1 and 35S:ATHB13. Transient transformation of sunflower, soybean and tobacco leaf disks was carried out as described for sunflower tissue (Manavella and Chan, 2009). For each construct, six disks originated from different plants were analyzed and the experiment repeated at least twice. As a control of the infiltration test, GUS reporter gene expression in these experiments was measured by histochemical assays.

In sunflower, four genes encoding one putative chitinase (Accession N^{o} TC18434), two putative transcription factors homologous to the Arabidopsis ZAT10 and SAG21 (Accession N^{o} of the sunflower genes TC16546 and TC19654 respectively) and one DREB-like (Accession N^{o} TC23839) transcription factor were analyzed.

The expression of three genes homologous to Arabidopsis glucanase, PR2 and PR4 was quantified in soybean transiently transformed leaf discs. All of them showed a significant increase in their levels when the transformation was performed with 35S:HAHB1 or 35:ATHB13; however with the second construct PR2 levels increased to a lesser extent. The experiment performed in tobacco leaf disks showed similar results but the inductions in the transformed disks were lower.

Since the Arabidopsis homologous genes were also induced in transgenic plants, this result indicates that there is a conserved mechanism of action in these species controlled by HD-Zip transcription factors, HAHB1 and its homologues.

The antifreeze mechanism mediated by HAHB1/ATHB13 seems to be conserved between other plant species like tobacco and soybean. The transient transformation of leaves from these plants induced the expression of HAHB1-target homologues, indicating that a homologue gene in these species is exerting the same type of regulation, probably conferring a similar tolerant phenotype.

### Example 16

### Expression of HAHB1 in Arabidopsis Confers Increased Tolerance to Pseudomonas Infection

Infection with Pseudomonas

An isolated colony of Pseudomonas syringae spp. was inoculated in LB medium supplemented with ryfampicine and cultured overnight at 28° C. The culture (2 ml) was centrifuged for 5 min at 4500 rpm and the cellular pellet washed three times with sterile water and finally suspended ( 1/50) in 10 mM MgCl₂, 15 μl/l Triton X-100. The diluted bacterial suspension was sprayed on 4-week-old Arabidopsis plants from different genotypes as indicated in the Figure legend. The tray was covered with nylon and the plants were photographed 2 days after the infection.

Staining with Evans blue was carried out essentially as described by Kato et al., 2007. Excised leaves were vacuum infiltrated with 0.1% Evans blue twice for 5 min. After that the leaves were washed three times with distilled water applying vacuum (10 min each time) until they were fully decolorized and then visualized on a microscope and photographed.

### REFERENCES

Arce A. L., Cabello J. V., Chan R. L. 2008. Patents on Plant Transcription Factors. Recent Patents on Biotechnology, 2: 209-217
Ariel F. D., Manavella P. A., Dezar C. A., Chan R. L. 2007. The true story of the HD-Zip family. Trends Plant Sci. 12, 419-426.
Cabello J. V., Dezar C. A., Manavella P. A., Chan R. L. 2007.
The intron of the Arabidopsis thaliana COX5c gene is able to improve the drought tolerance conferred by the sunflower Hahb-4 transcription factor. Planta 226, 1143-1154.
Carabelli M., Sessa G., Baima S., Morelli G., Ruberti I. 1993. The Arabidopsis Athb-2 and -4 genes are strongly induced by far-red-rich light. Plant J. 4: 469-479.
Chan R. L., Gago G. M., Palena C. M., Gonzalez D. H. 1998. Homeoboxes in plant development. Biochim. Biophys. Acta 1442: 1-19.
Chan R. L., Gonzalez D. H. 1994. A cDNA encoding an HD-Zip protein from sunflower. Plant Physiol. 106: 1687-1688.
Chinnusamy V., Zhu J., Zhu J. K. 2007. Cold stress regulation of gene expression in plants. Trends Plant Sci. 12: 444-451.
Clough S. J., Bent A. F. 1998. Floral dip: a simplified method for Agrobacterium-mediated transformation of Arabidopsis thaliana. Plant J. 16, 735-743.
DeVries A. L. 1986. Antifreeze Glycopeptides and Peptides: Interactions with Ice and Water, Methods In Enzymology, vol. 127, 293-303
Dezar C. A., Gago G. M., González D. H., Chan R. L. 2005. Hahb-4, a sunflower homeobox-leucine zipper gene, confers drought tolerance to Arabidopsis thaliana plants. Transgenic Res., 14, 429-440.
Dezar C. A., Fedrigo G. V., Chan R. L. 2005. The promoter of the sunflower HD-Zip protein gene Hahb4 directs tissue-specific expression and is inducible by water stress, high salt concentrations and ABA. Plant Sci. 169: 447-459.
Gago G. M., Almoguera C., Jordano J., González D. H., Chan R. L. 2002. Hahb-4, a homeobox-leucine zipper gene potentially involved in ABA-dependent responses to water stress in sunflower. Plant Cell Environ. 25: 633-640.
Gonzalez D. H., Valle E. M., Gago G. M., Chan R. L. 1997. Interaction between proteins containing homeodomains associated to leucine zippers from sunflower. Biochim. Biophys. Acta 1351: 137-149.
Griffith M., Yaish M. W. F. 2004. Antifreeze proteins in overwintering plants: a tale of two activities Trends Plant Sci. 9: 399-405.
Griffith M., Ala P., Yang D. S., Hon W. C., Moffatt B. A. 1992. Antifreeze Protein Produced Endogenously in Winter Rye Leaves. Plant Physiol. 100: 593-596.
Griffith, 2005. Antifreeze proteins modify the freezing process in planta. Plant Physiol. 138:330-340
Guy C. L., Huber J. L. A., Huber S. C. 1992. Sucrose phosphate synthase and sucrose accumulation at low temperature. Plant Physiol. 100: 502-508
Henriksson E., Olsson A. S., Johannesson H., Johansson H., Hanson J., Engstrom P., Soderman E. 2005. Homeodomain leucine zipper class I genes in Arabidopsis. Expression patterns and phylogenetic relationships. Plant Physiol. 139: 509-518.
Lee Y. H., Chun J. Y. 1998. A new homeodomain-leucine zipper gene from Arabidopsis thaliana induced by water stress and abscisic acid treatment. Plant Mol. Biol. 37: 377-384.
Levitt J. 1980. Responses of plants to environmental stresses. Academic Press. Vol I and II.
Manavella P. A., Arce A. L., Dezar C. A., Bitton F., Renou J. P., Crespi M., Chan R. L. 2006. Cross-talk between ethylene and drought signaling pathways is mediated by the sunflower Hahb-4 transcription factor. Plant J. 48: 125-137.
Manavella P. A., Dezar C. A., Bonaventure G., Baldwin I. T., Chan R. L. 2008 HAHB4, a sunflower HD-Zip protein, integrates signals from the jasmonic acid and ethylene pathways during wounding and biotic stress responses, Plant J. 56:376-388.
Mauch F., Staehelin L. A. 1989. Functional implications of the subcellular localization of ethylene-induced chitinase and [beta]-1,3-glucanase in bean leaves. Plant Cell. 1: 447-457.
Nanjo T., Kobayashi M., Yoshiba Y., Kakubari Y., Yamaguchi-Shinozaki K., Shinozaki K. 1999. Antisense suppression of proline degradation improves tolerance to freezing and salinity in Arabidopsis thaliana. FEBS Lett. 461:205-10.
Palena C. M., Gonzalez D. H., Chan R. L. 1999. A monomer-dimer equilibrium modulates the interaction of the sunflower homeodomain leucine-zipper protein Hahb-4 with DNA. Biochem. J. 341: 81-87.
Schena M., Davis R. W. 1992. HD-Zip protein members of Arabidopsis homeodomain protein superfamily. Proc. Natl. Acad. Sci. USA 89: 3894-3898.
Schena M., Davis R. W. 1994. Structure of homeobox-leucine zipper genes suggests a model for the evolution of gene families. Proc. Natl. Acad. Sci. USA 91: 8393-8397.
Schena M., Lloyd A. M., Davis R. W. 1993. The HAT4 gene of Arabidopsis encodes a developmental regulator. Genes Dev. 7: 367-379.
Schneiter A. A., Miller J. F. 1981. Description of sunflower growth stages. Crop Science 21: 901-903.
Sessa G., Ruberti I., Morelli G. 1997. DNA-binding specificity of the homeodomain-leucine zipper domain. J. Mol. Biol. 5:303-309.
Sessa G., Carbelli M., Ruberti, I. 1994. Identification of distinct families of HD-Zip proteins in Arabidopsis thaliana. In: Molecular-Genetic Analysis of Plant Development and Metabolism. G. Coruzzi and P. Puigdomenech (Eds.). Springer Verlag, Berlin.
Sessa G., Morelli G., Ruberti I. 1993. The Athb-1 and -2 HD-Zip domains homodimerize forming complexes of different DNA binding specificities. EMBO J. 12: 3507-3517.
Söderman E., Hjellstrom M., Fahleson J., Engstrom P. 1999. The HD-Zip gene ATHB6 in Arabidopsis is expressed in developing leaves, roots and carpels and up-regulated by water deficit conditions. Plant Mol. Biol. 40: 1073-1083.
Söderman E., Hjellström M., Fahleson J., Engström P. 1999. The HD-Zip gene ATHB6 in Arabidopsis is expressed in developing leaves, roots and carpels and up-regulated by water deficit conditions. Plant Mol. Biol. 40: 1073-1083.
Söderman E., Mattsson J., Engström P. 1996. The Arabidopsis homeobox gene ATHB-7 is induced by water deficit and by abscisic acid. Plant J. 10: 375-381.
Söderman E., Mattsson J., Svenson M., Borkird C., Engström P. 1994. Expression patterns of novel genes encoding homeodomain leucine-zipper proteins in Arabidopsis thaliana. Plant Mol. Biol. 26: 145-154.
Sukumaran N. P., Weiser C. J. 1972. Freezing Injury in Potato Leaves, Plant Physiol. 50: 564-567
Thomashow M. E. 1999. Plant cold acclimation: freezing tolerance genes and regulatory mechanisms. Annu. Rev. Plant Biol., 50: 571-599.
Tomczak M. M., Marshall C. B., Gilbert J. A., Davies P. L. 2003. A facile method for determining ice recrystallization inhibition by antifreeze proteins. Biochem Biophys Res Commun. 311: 1041-1046.
Waterhouse A. M., Procter J. B., Martin D. M. A Clamp M., Barton G. J. 2009. Jalview Version 2—a multiple sequence alignment editor and analysis workbench. Bioinformatics. 25:1189-1191
Whatley F. R., Tagawa K., Amon D. I. 1963. Separation of the Light and Dark Reactions in Electron Transfer during Photosynthesis. Proceedings of the National Academy of Sciences USA 49: 266-270.

### Sequence Listing Information

SEQUENCE LISTING
HAHB1 promoter - SEQ ID. 1
gtcgagctcgtctcgtaaaatgttcgagtcagctccaattaaatcatgtcggcttgtta
tattttttttaatttatttttgatattttttacatatatttataacataaaaaacaaaa
taaaaataaaaattacacatatatctatatgtattatttttctaaaattttaaatagcg
aaagacatattaaaagtattatatgtataattttgtttagcttcccatatttttatatg
ttattaattaattaaacttaaaatgttaacactttaacacctcttacatactttttagt
tcaatacatttaaaattaaaattaatctatatgcaataaaataattcaagcaggcttgc
aagctcacgagtcgagccatgcctggctcgagctcgactcatttacaaatcgagccacc
aagccgactcgtttataaccgagttttttttagccgagtttttttcaagcgaacttcaa
acaagtcacgcgcttttaattaacacattctagtctaaagaagataattgaaagagaaa
gtagatataagtaaaaggagtagccaaagatataaatttagggtctaaacaacctaata
ttgtttaattttttttaaataaactagtttttttttaccgattatctgtgttatatgtc
ttagtttgacatgataagttatcataattacttgtagtatttttatatcagaaatatac
gttggagaattaaattttatcctgatcgtcaattgacaagaacaaaaatcaacatctca
tggttttttactaatttatatgattaaagatatatggttgtaagaaaaagaacaatgta
catcaaatggtgaaatttgaatatttgatagtaacgtaatccattgtgtatttcttatt
attttatcattttcccaaggtgtgtcatatatagtgtctccattctttctatagcacaa
tatccttcacctccctctctctctctctctctctaaaaatgatgatgagacgacaaaga
tcgaattc
HAHB1 mRNA - SEQ. ID. 2
uugaaauucugagaaaagccacauaaucaaagcuaaagaggugguuuaaacagcugAUG
ACUUGCACUGGAAUGGCUUUCUUCUCCUCCAAUUUCAUGUUACAAUCCUCCCAAGAAGA
UGACCAUCAUGCCCCUACAUCUCUCUCUCCAAUCCUCCCACCUUGCAGUACCACCACUC
AAGAUUUCAGUGGUGCUGCUUUCUUGGGAAAAAGAUCUAUGUCUUCUUACUCAGGUUUG
AACAACAACAACAUGGAUGGAUGUGAUCAAGAAGGGAACAUGAAUGGAGAAGAUGAGUU
AUCAGAUGAUGGAUCACAGCUUCUUGCAGGAGAGAAAAAGAGGAGAUUAAACAUGGAAC
AAGUGAAGACACUUGAGAGAAACUUUGAGUUAGGAAAUAAGCUUGAACCUGAGAGGAAA
AUGCAACUUGCAAGAGCACUUGGACUACAACCAAGACAGAUUGCUAUAUGGUUUCAAAA
CAGAAGAGCUAGAUGGAAAACUAAACAGUUGGAAAAAGACUAUGAUGCCCUCAAGAGAC
AGUUUGAAGCUGUUAAAGCUGAGAAUGAUUCACUCCAAUCUCAAAAUCAUAAACUUCAU
GCUGAGAUAAUGGCACUAAAAAAUAGGGAGCCAGCAGAACUAAUCAACCUCAACAUAAA
AGAAACAGAAGGAUCUUGCAGCAACCGAAGCGAAAACAGCUCUGAAAUCAAACUAGACA
UCUCAAGAACACCGGCUACCGAUAGCCCUUUAUCAUCACACCAUCAACACCAACACCAG
CCAAUACCUAAUCUUUUUCCAUCGUCGAAUAUCGAUAGGCCUAAUUCGAAUAACAUUGU
GGCGCAUCAACUUUUCCACAAUUCGUCAUCAAGGCCGGCAGAUCAUCAACUUCAUUGCC
ACAAACUCGAUCAAUCGAAUGCCAUUAAAGAAGAAUGUUUUAGCACAAUGUUUGUUGGU
AUGGAUGAUCAAUCAGGGUUUUGGCCAUGGUUGGAACAACCACAAUUCAAUUGAuggaa
ucaagaagcaaaaaagcaaaagaaaacgguacccgauucgccuucuuggcuuugguuug
auuauauuaaagauggagaucaucaaucuguuuguucucuaagcuuuaaauucuuguuu
uuugguacuuaaauuaauagaguaaaaauuagaagaaaaaacguauuauuauuuuaaau
ucaagauuaguguuu
Coding sequence: in upper case
5′ and 3′ untranslated regions: in lower case
Flanking nucleotides from first and second introns: in bold
and underlined
Intron 1 - SEQ ID. 3
aattaactcaccttaactaagttacttatgacaacatctctctcatagatcttgatgca
gcttgcattcatgagttgtgatgtacaactcattcatgcattagggtttcagttttttc
aaagttttttttttattttttcttctgtttcaagatcatgatgatgagttgtgctgaac
acttgaacagctcattgatgcattagggtttgttttagtttcaagttctttcttttctt
tcattttcatgcactaaatccatatgggcttgaagaaagtttgaatctttatatgttag
ttgatgatcttgatgcaggt
Intron 2 - SEQ ID. 4
gtaatattagtttgattgtttattgcatctatcaatcattagattctactctttacttg
atcacacagaaagtaactaaaccttttttcctaatgataacaatatttgttttgcaaat
ctaatggcaatcaaataaaagtttctggtaagcagccatgatctatttatttttcacta
tttgagtaagtttaaaagttgcatttatcctcactaattatatacaacactaaaataat
cattaaactgactgttataattactttccgtaaacggtatgccaaaacttaaaatgatt
aacaattttataagaatggaaagtaaaatcattacactatttcccatattagtcatgac
caaagtttgtttctttctgaagggcaaaagggtcaatatgcttatatgcagcatgggca
aaagaagtagagtgtatatcaaaattcatatctttattttcttttcaaagtttaggtaa
caaaaagaagaaattataaacgagtttgttacaattccacaagtacatgaagaaacaaa
atttgttagtatttttattttccatgtttttagtaacttccatatcaatttagcactag
aagataactttttttaggactcggtaaaccatacaagtagggtcatactttatcgttta
tccattaatgtatatccataaattcactgattatgcggtatttcctttgttacactgtc
ttgaacaagtattagtacatgtagtttcttaaagattgtttaatcaaccaaaaagattg
aaactttgcag
HAHB1 protein - SEQ ID. 5
MTCTGMAFFSSNFMLQSSQEDDHHAPTSLSPILPPCSTTTQDFSGAAFLGKRSMSSYSG
LNNNNMDGCDQEGNMNGEDELSDDGSQLLAGEKKRRLNMEQVKTLERNFELGNKLEPER
KMQLARALGLQPRQIAIWFQNRRARWKTKQLEKDYDALKRQFEAVKAENDSLQSQNHKL
HAEIMALKNREPAELINLNIKETEGSCSNRSENSSEIKLDISRTPATDSPLSSHHQHQH
QPIPNLFPSSNIDRPNSNNIVAHQLFHNSSSRPADHQLHCHKLDQSNAIKEECFSTMFV
GMDDQSGFWPWLEQPQFN
HAHB1 cDNA- SEQ ID. 6
ttgaaattctgagaaaagccacataatcaaagctaaagaggtggtttaaacagctgATG
ACTTGCACTGGAATGGCTTTCTTCTCCTCCAATTTCATGTTACAATCCTCCCAAGAAGA
TGACCATCATGCCCCTACATCTCTCTCTCCAATCCTCCCACCTTGCAGTACCACCACTC
AAGATTTCAGTGGTGCTGCTTTCTTGGGAAAAAGATCTATGTCTTCTTACTCAGGTTTG
AACAACAACAACATGGATGGATGTGATCAAGAAGGGAACATGAATGGAGAAGATGAGTT
ATCAGATGATGGATCACAGCTTCTTGCAGGAGAGAAAAAGAGGAGATTAAACATGGAAC
AAGTGAAGACACTTGAGAGAAACTTTGAGTTAGGAAATAAGCTTGAACCTGAGAGGAAA
ATGCAACTTGCAAGAGCACTTGGACTACAACCAAGACAGATTGCTATATGGTTTCAAAA
CAGAAGAGCTAGATGGAAAACTAAACAGTTGGAAAAAGACTATGATGCCCTCAAGAGAC
AGTTTGAAGCTGTTAAAGCTGAGAATGATTCACTCCAATCTCAAAATCATAAACTTCAT
GCTGAGATAATGGCACTAAAAAATAGGGAGCCAGCAGAACTAATCAACCTCAACATAAA
AGAAACAGAAGGATCTTGCAGCAACCGAAGCGAAAACAGCTCTGAAATCAAACTAGACA
TCTCAAGAACACCGGCTACCGATAGCCCTTTATCATCACACCATCAACACCAACACCAG
CCAATACCTAATCTTTTTCCATCGTCGAATATCGATAGGCCTAATTCGAATAACATTGT
GGCGCATCAACTTTTCCACAATTCGTCATCAAGGCCGGCAGATCATCAACTTCATTGCC
ACAAACTCGATCAATCGAATGCCATTAAAGAAGAATGTTTTAGCACAATGTTTGTTGGT
ATGGATGATCAATCAGGGTTTTGGCCATGGTTGGAACAACCACAATTCAATTGAtggaa
tcaagaagcaaaaaagcaaaagaaaacggtacccgattcgccttcttggctttggtttg
attatattaaagatggagatcatcaatctgtttgttctctaagctttaaattcttgttt
tttggtacttaaattaatagagtaaaaattagaagaaaaaacgtattattattttaaat
tcaagattagtgttt
Coding sequence: in upper case
5′ and 3′ untranslated regions: in lower case
Flanking nucleotides from first and second introns: in bold
and underlined
HAHB1 gene sequence - SEQ ID. 7
gtcgagctcgtctcgtaaaatgttcgagtcagctccaattaaatcatgtcggcttgtta
tattttttttaatttatttttgatattttttacatatatttataacataaaaaacaaaa
taaaaataaaaattacacatatatctatatgtattatttttctaaaattttaaatagcg
aaagacatattaaaagtattatatgtataattttgtttagcttcccatatttttatatg
ttattaattaattaaacttaaaatgttaacactttaacacctcttacatactttttagt
tcaatacatttaaaattaaaattaatctatatgcaataaaataattcaagcaggcttgc
aagctcacgagtcgagccatgcctggctcgagctcgactcatttacaaatcgagccacc
aagccgactcgtttataaccgagttttttttagccgagtttttttcaagcgaacttcaa
acaagtcacgcgcttttaattaacacattctagtctaaagaagataattgaaagagaaa
gtagatataagtaaaaggagtagccaaagatataaatttagggtctaaacaacctaata
ttgtttaattttttttaaataaactagtttttttttaccgattatctgtgttatatgtc
ttagtttgacatgataagttatcataattacttgtagtatttttatatcagaaatatac
gttggagaattaaattttatcctgatcgtcaattgacaagaacaaaaatcaacatctca
tggttttttactaatttatatgattaaagatatatggttgtaagaaaaagaacaatgta
catcaaatggtgaaatttgaatatttgatagtaacgtaatccattgtgtatttcttatt
attttatcattttcccaaggtgtgtcatatatagtgtctccattctttctatagcacaa
tatccttcacctccctctctctctctctctctctaaaaatgatgatgagacgacaaaga
tcgaattcttgaaattctgagaaaagccacataatcaaagctaaagaggtggtttaaac
agctgATGACTTGCACTGGAATGGCTTTCTTCTCCTCCAATTTCATGTTACAATCCTCC
CAAGAAGATGACCATCATGCCCCTACATCTCTCTCTCCAATCCTCCCACCTTGCAGTAC
CACCACTCAAGATTTCAGTGGTaattaactcaccttaactaagttacttatgacaacat
ctctctcatagatcttgatgcagcttgcattcatgagttgtgatgtacaactcattcat
gcattagggtttcagttttttcaaagttttttttttattttttcttctgtttcaagatc
atgatgatgagttgtgctgaacacttgaacagctcattgatgcattagggtttgtttta
gtttcaagttctttcttttctttcattttcatgcactaaatccatatgggcttgaagaa
agtttgaatctttatatgttagttgatgatcttgatgcaggtGCTGCTTTCTTGGGAAA
AAGATCTATGTCTTCTTACTCAGGTTTGAACAACAACAACATGGATGGATGTGATCAAG
AAGGGAACATGAATGGAGAAGATGAGTTATCAGATGATGGATCACAGCTTCTTGCAGGA
GAGAAAAAGAGGAGATTAAACATGGAACAAGTGAAGACACTTGAGAGAAACTTTGAGTT
AGGAAATAAGCTTGAACCTGAGAGGAAAATGCAACTTGCAAGAGCACTTGGACTACAAC
CAAGACAGATTGCTATATGGTTTCAAAACAGAAGAGCTAGATGGAAAACTAAACAGTTG
GAAAAAGACTATGATGCCCTCAAGAGACAGTTTGAAGCTGTTAAAGCTGAGAATGATTC
ACTCCAATCTCAAAATCATAAACTTCATGCTGgtaatattagtttgattgtttattgca
tctatcaatcattagattctactctttacttgatcacacagaaagtaactaaacctttt
ttcctaatgataacaatatttgttttgcaaatctaatggcaatcaaataaaagtttctg
gtaagcagccatgatctatttatttttcactatttgagtaagtttaaaagttgcattta
tcctcactaattatatacaacactaaaataatcattaaactgactgttataattacttt
ccgtaaacggtatgccaaaacttaaaatgattaacaattttataagaatggaaagtaaa
atcattacactatttcccatattagtcatgaccaaagtttgtttctttctgaagggcaa
aagggtcaatatgcttatatgcagcatgggcaaaagaagtagagtgtatatcaaaattc
atatctttattttcttttcaaagtttaggtaacaaaaagaagaaattataaacgagttt
gttacaattccacaagtacatgaagaaacaaaatttgttagtatttttattttccatgt
ttttagtaacttccatatcaatttagcactagaagataactttttttaggactcggtaa
accatacaagtagggtcatactttatcgtttatccattaatgtatatccataaattcac
tgattatgcggtatttcctttgttacactgtcttgaacaagtattagtacatgtagttt
cttaaagattgtttaatcaaccaaaaagattgaaactttgcagAGATAATGGCACTAAA
AAATAGGGAGCCAGCAGAACTAATCAACCTCAACATAAAAGAAACAGAAGGATCTTGCA
GCAACCGAAGCGAAAACAGCTCTGAAATCAAACTAGACATCTCAAGAACACCGGCTACC
GATAGCCCTTTATCATCACACCATCAACACCAACACCAGCCAATACCTAATCTTTTTCC
ATCGTCGAATATCGATAGGCCTAATTCGAATAACATTGTGGCGCATCAACTTTTCCACA
ATTCGTCATCAAGGCCGGCAGATCATCAACTTCATTGCCACAAACTCGATCAATCGAAT
GCCATTAAAGAAGAATGTTTTAGCACAATGTTTGTTGGTATGGATGATCAATCAGGGTT
TTGGCCATGGTTGGAACAACCACAATTCAATTGAtggaatcaagaagcaaaaaagcaaa
agaaaacggtacccgattcgccttcttggctttggtttgattatattaaagatggagat
catcaatctgtttgttctctaagctttaaattcttgttttttggtacttaaattaatag
agtaaaaattagaagaaaaaacgtattattattttaaattcaagattagtgttt
Coding sequence: in upper case
Promoter region: lower case
5′ and 3′ untranslated regions: in underlined lower case
First and second introns: in bold lower case
C-terminus of HAHB1 SEQ ID NO. 8
LINLNIKETEGSCSNRSENSSEIKLDISRTPATDSPLSSHHQHQHQPIPNLFPSSNIDR
PNSNNIVAHQLFHNSSSRPADHQLHCHKLDQSNAIKEECFSTMFVGMDDQSGFWPWLEQ
PQFN
HAHB1 C-terminus motif I (CI) SEQ ID NO. 9
LINLNIKETEGSCSNRSENSSEIKLDISRTPATDS
HAHB1 C-terminus motif II (CII) SEQ ID NO. 10
IKEECFSTMFVGMDDQSGFWPWLEQPQFN
Consensus sequence for the conserved region adjacent to the
leucine zipper (CI, 34 aminoacids) SEQ ID NO. 11
SINLNKETEGSCSNRSENSSDIKLDISRTPAIDS
Consensus sequence for the second conserved region located
at the C-terminal end (CII, 29 aminoacids) SEQ ID NO. 12
VKEESLSNMFCGIDDQSGFWPWLEQQHFN
N-terminus of HAHB1 SEQ ID NO. 13
MTCTGMAFFSSNFMLQSSQEDDHHAPTSLSPILPPCSTTTQDFSGAAFLGKRSMSSYSG
LNNNNMDGCDQEGNMNGEDELSDDGSQLLAGE
N-terminal homeodomain consensus sequence SEQ ID NO. 14
KKRRLTDEQVKALEKSFELENKLEPERKVQLARELGLQPRQVAVWFQNRRARWKTKQ

| Primer | | | Sequence | |
|---|---|---|---|---|
| name | Primer sequence | SEQ ID No. | information | Used for |
| PrH1R | 5′ CGGGGATCCCCTCTTTAGCTT | 15 | 2 | Cloning |
| | TGATTATGTGGC 3′ | | | HAHB1 |
| | | | | promoter |
| PR-TOPO | 5′ AACAGCTATGACCATG 3′ | 16 | | Cloning |
| | | | | HAHB1 |
| | | | | promoter |
| H1qF | 5′ GGCCGGCAGATCATCAACTTC 3′ | 17 | 2 | Real Time |
| | | | | PCR |
| H1qR | 5′ CCAACCATGGCCAAAACCCT | 18 | 2 | Real Time |
| | G 3′ | | | PCR |
| PR4 CDS- | 5′ GGCGGATCCCCACCAAGAAA | 19 | | Cloning |
| F | ACAAAGACTTAT 3′ | | | At3g04720 |
| PR4 CDS- | 5′ GGGGAGCTCCCGATCGATATT | 20 | | Cloning |
| R | GACCTC 3′ | | | At3g04720 |
| PR2 CDS- | 5′ GGCGGATCCAAGAAAATGTC | 21 | | Cloning |
| F | TGAATCAAGG 3′ | | | At3g57260 |
| PR2 CDS- | 5′ GGGGAGCTCGCCCACAAGTC | 22 | | Cloning |
| R | TCTAAGG 3′ | | | At3g57260 |
| Glucanase | 5′ CGCGGATCCCTAAGGAGCTA | 23 | | Cloning |
| CDS-F | AGAACAAACCC 3′ | | | At4g16260 |
| Glucanase | 5′ CCCGAGCTCATCACTCAACCG | 24 | | Cloning |
| CDS-R | CCGTACCG 3′ | | | At4g16260 |
| tc23839-F | 5′AAAAGTGGTTTATTTGGAT | 25 | Sunflower | Real Time |
| | GAGGA 3′ | | gene | PCR |
| tc23839-R | 5′GACACGTCAGAACAAAATT | 26 | similar to | Real Time |
| | CCA 3′ | | DREB 1A | PCR |
| | | | and 1B | |
| | | | from | |
| | | | Arabidopsis | |
| tc19654-F | 5′GATCTTCGTGACCTGCTTCT | 27 | Sunflower | Real Time |
| | AAAAC 3′ | | gene | PCR |
| tc19654-R | 5′CAAACCACTTCTAAATCATC | 28 | similar to | Real Time |
| | CCATAG 3′ | | SAG21 | PCR |
| | | | from | |
| | | | Arabidopsis | |
| tc16546-F | 5′CAAAACAACTTCTTCCACC | 29 | Sunflower | Real Time |
| | AATAGTC 3′ | | gene | PCR |
| tc16546-R | 5′AATAAACCGTTGACTTTTCT | 30 | similar to | Real Time |
| | TCACC 3′ | | ZAT10 | PCR |
| | | | from | |
| | | | Arabidopsis | |
| tc18434-F | 5′ACATCATCAACGGTGGTTT | 31 | Sunflower | Real Time |
| | AGAAT 3′ | | gene | PCR |
| tc18434-R | 5′ACATGGTGCAATACCTTCTG | 32 | similar to | Real Time |
| | TAAAA 3′ | | Quitinase | PCR |
| | | | from | |
| | | | Arabidopsis | |
| At3g12500- | 5′GGGTTATGGAGTGATTACG | 33 | PR3 from | Real Time |
| F | AACAT 3′ | | Arabidopsis | PCR |
| At3g12500- | 5′ TACCACCAGGATTAACACC | 34 | | Real Time |
| R | AAATA 3′ | | | PCR |
| At3g57260- | 5′TAAGAAGGAACCAACGTAT | 35 | β-1,3 | Real Time |
| F | GAGAA 3′ | | Glucanase | PCR |
| At3g57260- | 5′CATAAAAAGCCCACAAGTC | 36 | from | Real Time |
| R | TCTAA 3′ | | Arabidopsis | PCR |
| At3g04720- | 5′ATTGAACATTGCTACATCCA | 37 | PR4 from | Real Time |
| F | AATC 3′ | | Arabidopsis | PCR |
| At3g04720- | 5′ATTGAACATTGCTACATCCA | 38 | | Real Time |
| R | AATC 3′ | | | PCR |

Used for micro-array validation

| | | | | |
|---|---|---|---|---|
| At1g62440- | 5′ ACCAACACCACCTTCTCTGC 3′ | 39 | LRX1 | Real |
| F | | | | Time |
| | | | | PCR |
| At1g62440- | 5′ TTGATGGTGGAGGAGGAGAC 3′ | 40 | LRX1 | Real |
| R | | | | Time |
| | | | | PCR |
| At2g43050- | 5′ GGTTAAATGGAGTGGGTGTCAT 3′ | 41 | Cell wall | Real |
| F | | | modificator | Time |
| | | | | PCR |
| At2g43050- | 5′ CAAGTCCTGGGTCGAAACTAAC 3′ | 42 | | Real |
| R | | | | Time |
| | | | | PCR |
| At4g 16260- | 5′ GAGACCTGGAAGAGGAGTGGAAAC 3′ | 43 | Glucanase | Real |
| F | | | | Time |
| | | | | PCR |
| At4g 16260- | 5′ AATGTGATCGGAAATTTTGGTTGT 3′ | 44 | Glucanase | Real |
| R | | | | Time |
| | | | | PCR |
| At4g25480- | 5′ GACGTTGGTGGAGGCTATTTACAC 3′ | 45 | DREB 1b | Real |
| F | | | | Time |
| | | | | PCR |
| At4g25480- | 5′ TATTAGCCAACAAACTCGGCATCT 3′ | 46 | DREB 1b | Real |
| R | | | | Time |
| | | | | PCR |
| At4g02380- | 5′ ATGCTATCTTCCGACGTGGTTATG 3′ | 47 | SAG21 | Real |
| F | | | | Time |
| | | | | PCR |
| At4g02380- | 5′ CTTCCACTCCCTTCTTCTTCATCA 3′ | 48 | SAG21 | Real |
| R | | | | Time |
| | | | | PCR |
| At4g25490- | 5′ CGTTGGCTTTTCAAGATGAGAC 3′ | 49 | DREB1a | Real |
| F | | | | Time |
| | | | | PCR |
| At4g25490- | 5′ CGCTCTGTTCCGGTGTATAAATAG 3′ | 50 | DREB1a | Real |
| R | | | | Time |
| | | | | PCR |
| At1g27730- | 5′ GTCCACTAGCCACGTTAGCAGTA 3′ | 51 | ZAT10 | Real |
| F | | | | Time |
| | | | | PCR |
| At1g27730- | 5′ AGTTGAAGTTTGACCGGAAAGTC 3′ | 52 | ZAT10 | Real |
| R | | | | Time |
| | | | | PCR |
| At3g04720- | 5′ ATTGAACATTGCTACATCCAAATC 3′ | 53 | PR4 | Real |
| F | | | | Time |
| | | | | PCR |
| At3g12500- | 5′ GGGTTATGGAGTGATTACGAACAT 3′ | 54 | PR3 | Real |
| F | | | | Time |
| | | | | PCR |
| At3g12500- | 5′ TACCACCAGGATTAACACCAAATA 3′ | 55 | PR3 | Real |
| R | | | | Time |
| | | | | PCR |
| At3g52130- | 5′ TTTCTCTTTAATAACCTTGCTGCTT 3′ | 56 | LBP | Real |
| F | | | | Time |
| | | | | PCR |
| At3g52130- | 5′ GCTAATGACTGAGATTTTGATTCG 3′ | 57 | LBP | Real |
| R | | | | Time |
| | | | | PCR |
| At3g07450- | 5′ GACACTTGGTCAACCTTGTTTATG 3′ | 58 | LTP | Real |
| F | | | | Time |
| | | | | PCR |
| At3g07450- | 5′ TACATGGAAGAAAATTGGCAGAAC 3′ | 59 | LTP | Real |
| R | | | | Time |
| | | | | PCR |
| At5g44420- | 5′ CTTGTTCTCTTTGCTGCTTTCGACG 3′ | 60 | PDF1.2 | Real |
| F | | | | Time |
| | | | | PCR |
| At5g44420- | 5′ CTTCAAGGTTAATGCACTGATTCT 3′ | 61 | PDF1.2 | Real |
| R | | | | Time |
| | | | | PCR |
| At3g57260- | 5′ TAAGAAGGAACCAACGTATGAGAA 3′ | 62 | PR2 | Real |
| F | | | | Time |
| | | | | PCR |
| At3g57260- | 5′CATAAAAAGCCCACAAGTCTCTAA 3′ | 63 | PR2 | Real |
| R | | | | Time |
| | | | | PCR |

SEQ ID NO. 64: ATHB13
ACCAGAAGTGGTATAGTCTAGGCCGATACATTTCACTATCTCTCTCTCTTTTGTTTTTC
CTCTTCTTCTTTTTTTCCATTTGATTTCAAACTCTCACACAAAGAGCTTCAGATTTATA
AGACCATGATAATGGCTTTAAGACAAAGATTGGCAAGAAGAAAAAACTAAAGAGAAACG
ACCAAAATCTCAAGCAAACAGTACTAACTTCTGTTGCAAAACAGAAGAAGATGTCTTGT
AATAATGGAATGTCTTTTTTCCCTTCAAATTTCATGATCCAAACCTCTTACGAAGATGA
TCATCCTCATCAATCTCCATCTCTTGCTCCTCTTCTTCCTTCTTGCTCTCTACCTCAAG
ATCTCCATGGTATATATACATAAACTTCCACACACATCTCCTCTGTTTTCTCTCTATCT
CTTTCTAATGCTCTGTTCTGTTCTGTTTCAGGATTTGCTTCGTTTCTAGGTAAGAGATC
TCCAATGGAAGGGTGTTGTGATTTAGAAACAGGGAACAATATGAATGGAGAAGAGGATT
ATTCAGATGATGGGTCACAAATGGGAGAGAAGAAGAGGAGATTGAACATGGAACAAGTG
AAGACACTAGAGAAGAACTTTGAGCTTGGAAACAAACTTGAACCAGAGAGGAAAATGCA
GCTAGCTCGTGCCTTAGGTTTGCAACCAAGACAGATCGCGATTTGGTTTCAAAATCGAA
GAGCTCGTTGGAAAACAAAGCAGCTAGAGAAAGATTATGATACTCTTAAACGACAGTTT
GATACACTTAAAGCTGAAAATGATCTTCTTCAAACTCATAATCAGAAACTCCAAGCTGA
GGTAATTAATCTCATAAATTAACAAAAAAAATCAATATGTGTTATTTTTTTTTGGGTTA
ATGATCAATAATTACAGTTATTTTCCATCTAAAGGATGATTTTTTTCTTTTTAAAAAAG
GTTAAAAATTATATTTCTGGTTTATAATTATTTGGATCAGGAGTTGCTTTCAGGTAGGG
TTAAAAAACTGGACATGATTCATGACTTTTCAGACATCATTATCTCTTTTTTTCTTCAC
TCTTGTCTGGAAAGAGATCTGAAAACAATAGTTTCTTTATGCTTATCACATTGTACAGT
AACTCTGTTTATGTTTAAAATTTTGTCTTTAATTACGCAGATAATGGGATTAAAAAACA
GAGAACAAACAGAATCAATAAATCTAAACAAAGAAACTGAAGGATCTTGCAGTAACAGA
AGTGATAACAGTTCAGATAATCTCAGACTAGATATCTCAACTGCGCCGCCATCAAACGA
CAGTACATTAACCGGTGGCCACCCACCGCCACCACAGACAGTTGGTCGACACTTCTTCC
CACCGTCGCCAGCCACCGCAACGACAACTACTACAACAATGCAGTTCTTTCAAAACTCA
TCTTCAGGACAGAGTATGGTTAAAGAAGAGAATAGTATCAGTAACATGTTCTGTGCAAT
GGATGACCATTCTGGTTTTTGGCCATGGCTTGATCAGCAACAGTACAATTGAAATTGGT
CTACCTGTTTTTTTTGTTTTTGTTTTTAAAAAAATTTATATTTTTTTTTTTTGTATTTG
GAATTTTGATCAGAAGAACCCATGCATGTTTTCAAAAACTGGAATCTATATCATTAGCT
CACTTTGAAATCTGCAACCAAACACCACTGAGGTTTTTTGTTTACTTTTTGAGTAAATG
AGATGTAAAAAAATGGGTAATATCCATTATATTATATAAAAAATAATATCATTATGGCC
CAACATTTTTCTGTATGGAGAAAAATAAAATAAATTGTATATT
This sequence corresponds to the immature mRNA (including
introns and untranslated 3′ and 5′) as defined in the TAIR
database (www.Arabidopsis.org).
SEQ ID NO. 65 glucanase
ctactaaaaaaattgtaagtacatacatatcaatgttaatttgtatataaggagctaag
aacaaacccaattaggcaactagcaattgctaaaacacgtaagatctcaaatATGacca
cgttattcctccttattgctctattcatcacaaccatcctcaacccaacaagtctctct
ctctctctctttaatctcacatgatttattctcattttcaatttttatagatatataaa
cttaatcataattaaccttaattatgttatatgataaactaggtggagaatcagtaggt
gtatgctatggaatgatggggaacaaccttccttctcaatcagacacaatcgctctctt
tagacaaaacaacatccgacgtgttagactctacgatccaaaccaagccgctttaaacg
ctcttagaaacacgggtatcgaagtcatcatcggcgttccaaacaccgatcttcgttca
ctcactaacccttcttccgctagatcatggctccaaaacaacgtcctcaactattaccc
cgccgttagcttcaagtacatcgccgtaggtaacgaagtatctccgtcgaacggcggtg
atgttgtgctccctgccatgcgtaacgtttacgatgctctaagaggtgcaaatcttcaa
gatcgtattaaagtttctaccgccattgatatgactttgattggaaactctttccctcc
ttcctccggagagtttcgtggtgacgttagatggtatatcgatcccgtcatcgggtaat
tttccaaccaaaccaaataaccaaaattcattagatttagttatttcccaatatttttc
atttctggttacttgtggaatgattatttaatttcttcctatgtggctaattaggtttc
ttacgagtacgaactcagcgttactagccaacatctatccttacttcagctacgttgac
aatccacgtgacatatctctctcttacgctctcttcacttctccttccgtcgtcgtatg
ggacggctctcgtggctaccaaaacctctttgacgctttacttgacgttgtttactctg
ccgttgaacgctcaggcggtggatctctcccagtggttgtttccgagagcggatggcct
tctaacggtggaaacgccgcgagtttcgataacgcgcgaagctttttacacgaatcttg
cgtcgcgtgtgagagagaacagaggaacaccgaagagacctggaagaggagtggaaacg
tatttgttcgctatgtttgatgagaatcaaaagagtcctgagatcgagaagaattttgg
tttgttttttcctaataaacaaccaaaatttccgatcacattctctgccgcgagagacg
gtacggcggttgagtgatgattttatatgctgagatttatgtgaataattgggagatta
tcccataaaaggttccaaataaagacaaatttcaaataaaacctgttagtccaagttaa
attaaatactcggctttgttttggtccacgttagacttggtaaagtcatgcaatatttt
tatttgatat
ATG signaled in bold and capital letters
Introns in bold
Oligonucleotides used for cloning, underlined
SEQ ID NO. 66 PR2
atatcatttttcacagaatcatagaaaaatcaagaaaATGtctgaatcaaggagcttag
cctcaccaccaatgttgatgattcttctcagccttgtaatagcttccttcttcaaccac
acaggttcagtcatcttttaagctattgtaacatctattaatcatctccatcttcacaa
atttattcaatttaatgattcttattttggaaaatgaagctggacaaatcggagtatgc
tacgggatgctaggcgataccttgccaagtccatcggacgttgtggctctttacaaaca
acaaaacatccagcgaatgcggctctacggccctgacccaggcgctcttgccgctctcc
gtggctctgacatcgagctcatcctcgacgttcccagttcagatcttgaacgtctcgcc
tccagtcaaacggaggccgacaagtgggttcaagaaaacgttcagagctacagagatgg
tgtcagattccggtacatcaacgttggaaatgaggtgaaaccctcagttggggggtttc
tcttacaagcaatgcagaacatcgagaacgcggtttctggagcagggcttgaagtcaag
gtctcaacagctatagccactgacaccaccactgatacgtctcctccgtctcaaggaag
gttcagggatgagtataagagctttctcgaaccagtgataggtttcttggcaagcaagc
aatctcccttgctcgtgaatctctacccttacttcagctacatgggagacacggccaac
atccatctagactacgctctgttcaccgcccagtccactgttgataacgatccagggta
ctcataccaaaacctattcgacgcaaatctcgactcggtttatgcagcattggagaaat
cagggggcggatcgttggaaatcgtggtgtcggagaccggttggcccacagagggagca
gtcgggacgagtgtggaaaacgcaaagacttatgttaacaatttgatacaacatgtgaa
gaatggatcaccgagaaggccagggaaagctatagagacttatatattcgctatgttcg
atgagaataagaaggaaccaacgtatgagaagttttggggactgtttcatccagatcga
cagtctaagtatgaagttaatttcaactaatccttagagacttgtgggctttttatgta
agcgtatttaaaaattgggaacttgttgtagtaataaggaataattaatgcgctttcag
cgtgtagtatgttgttatttttaaggttataaatgagctgcaagcataaataaggaaaa
aaaatagcatgggcctataggcccaataataaaacaagcttgctt
ATG signaled in bold and capital letters
Introns in bold
Oligonucleotides used for cloning, underlined
SEQ ID No. 67
Genomic sequence of PR4 (At3g04720) encoding gene.
Underlined are the sequences of the oligonucleotides used
to amplified this gene from genomic DNA
agaccaccaagaaaacaaagacttatcgatcatgaagatcagacttagcataaccatca
tacttttatcatacacagtggctacggtggccggacaacaatgcggtcgtcaaggcggt
ggtcgaacttgtcccggtaacatctgctgcagtcagtacggttactgtggtaccaccgc
ggactactgttctccgaccaacaactgtcagagcaattgttggggaagtgggcctagcg
gaccaggggagagcgcgtcgaacgtacgcgccacctaccatttctataatccggcgcag
aataattgggatttgagagccgtgagtgcttattgctccacgtgggatgctgataagcc
gtacgcatggcggagcaagtatggctggaccgccttctgcgggccggcaggacctcgtg
gtcaagcttcttgcggcaagtgtttaagggtaagttaattaattatctttttctcaaat
ctttatataagtatgtttgtgcaaaaggagatcatatagaaagtgttggaattaagacg
aatacaagataaaatttgttaccatttaccaacgtcaacgtgttagtgaaatatttcaa
aagatgtatagccggtaaaaattgtgattaaccggtgggtataaatggattcaggtgaa
gaacacaagaacaaatgctgcagtaactgtgagaatagtggaccaatgcagcaacggag
gcttggatttggatgtagcaatgttcaatcaaatagacaccgatggttttggctatcaa
caaggccatctcattgttgactaccaatttgtcgactgtggcaatgagctcattgggca
gcctgattccagaaacatgcttgtttcggccattgatcgcgtttgatattatgtaatga
ttttgaggtcaatatcgatcggtctacataaaaataataaagaccgctatatatgtatt
gtcgagggatatatgtttcgtatcaataaggaaattttaaatattattatcatt
Oligonucleotides used to quantify putative targets in
Nicotiana and soybean
GMPR2 gi|210143170|dbj|AK285952.1| Glycine max cDNA, clone:
GMFLO1-19-B17
GmPR2qF
5′ CCTTCTTCTGGTGGAACTGC 3′
SEQ ID No. 68
GmPR2qR
5′ ATAGGAGAAAAGAGCCCCCA 3′
SEQ ID No. 69
NTPR2 gi|194719370|gb|EU867448.1| Nicotiana tabacum basic
beta-1,3-glucanase gene, complete cds
NtPR2qF
5′ CTGGTTTGGGAAACAACATCAA 3′
SEQ ID No. 70
NtPR2qR
5′ AATCTGGCCTGGATTACCAGAA 3′
SEQ ID No. 71
GMPR4 gi|210142121|dbj|AK246040.1| Glycine max cDNA, clone:
GMFL01-49-117
GmPR4qF
5′ ACAGGAACAGGAGCAAACACAA 3′
SEQ ID No. 72
GmPR4qR
5′ CATTCCCACAATCCACAAACTG 3′
SEQ ID No. 73
NTPR4 gi|632733|gb|S72452.1| Nicotiana tabacum pathogen-
and wound-inducible antifungal protein CBP20 (CBP20) mRNA,
complete cds
NtPR4qF
5′ TTTGGCATGGAGGAGGAAGTAT 3′
SEQ ID No. 74
NtPR4qR
5′ TCCACGATTCTCACTGTGGTCT 3′
SEQ ID No. 75
GM-glucanase gi|38640794|gb|AY461847.1| Glycine max endo-
1,3-beta-glucanase mRNA, complete cds
GmGlucqF
5′ GAGAAAGTAGGGGCACCAAATG 3′
SEQ ID No. 76
GmGlucqR
5′ TTCTGGTTTCCATCAAACATGG 3′
SEQ ID No. 77
GM elongation factor (used as internal control)
GmEF1aqF
5′ TGAAACAGATGATTTGCTGCTGTA 3′
SEQ ID No. 78
GmEF1aqR
5′ CAATCATGTTGTCTCCCTCAAAAC 3′
SEQ ID No. 79
Nt ACTIN (used as internal control)
NtActqF
5′ CTGATGGACAGGTTATCACCATTG 3′
SEQ ID No. 80
NtActqR
5′ TAATGCGGTAATTTCCTTGCTCAT 3′
SEQ ID No. 81

1. A transgenic plant expressing a transgene encoding for a protein of SEQ ID NO. 5 or a functional part of said protein.

2. A transgenic plant as defined in claim 1 wherein said plant has been transformed with a vector comprising a nucleic acid sequence of SEQ ID No. 2, SEQ ID No. 6, SEQ ID No. 7 or a nucleic acid encoding for SEQ ID No. 8.

3. A transgenic plant as defined in claim 1 wherein said plant shows enhanced stress tolerance compared to a wild type plant.

4. A transgenic plant as defined in claim 3 wherein said plant shows enhanced freezing tolerance, enhanced low-temperature tolerance, enhanced chilling tolerance, enhanced tolerance to drought, enhanced tolerance to conditions of high salinity and/or enhanced pathogen resistance.

5. A method for producing a stress tolerant plant or enhancing stress tolerance comprising transforming a plant with a nucleic acid sequence of SEQ ID No. 2, 6 or 7, a functional part or a functional variant thereof.

6. A method according to claim 5 wherein said functional part is a nucleic acid sequence encoding for SEQ ID No. 8.

7. A method according to claim 5 wherein said stress tolerance is selected from freezing tolerance, enhanced low-temperature tolerance, enhanced chilling tolerance, enhanced tolerance to drought, enhanced tolerance to conditions of high salinity and/or enhanced pathogen resistance.

8. A method as defined in claim 7 comprising transforming a plant with a nucleic acid sequence encoding for ATHB13 as defined in SEQ ID NO. 64.

9. A plant obtainable or obtained by a method as defined in claim 5.

10. An isolated nucleic acid sequence consisting of SEQ ID No. 8, 9, 10 or 13.

11. An isolated chimeric nucleic acid construct comprising a nucleic acid sequence encoding for the N-terminal sequence of an HD Zip protein of subfamily I or a sequence comprising a consensus motif or part thereof operatively associated with a nucleic acid sequence encoding for a sequence comprising the C terminus of HAHB1, or a sequence comprising a consensus motif or part thereof.

12. A polypeptide encoded by a nucleic acid construct of claim 11.

13. A polypeptide of claim 12 wherein said C-terminal sequence comprises SEQ ID NO. 8, 9, 10, 11 or 12.

14. A polypeptide of claim 12 wherein said N-terminal sequence comprises a sequence with homology to the consensus sequence of SEQ ID NO. 14.

15. A polypeptide of claim 14 wherein said homology is at least 80%, preferably at least 90%, more preferably at least 95%.

16. A polypeptide of claim 15 wherein the N-terminal sequence of an HD Zip protein of subfamily I is operatively associated with a sequence comprising a sequence with homology to the C-terminal consensus motif as defined in SEQ ID NO. 11 and/or the C-terminal consensus motif as defined in SEQ ID NO. 12.

17. A polypeptide of claim 16 wherein said homology is at least 80%, preferably at least 90%, more preferably at least 95%.

18. A polypeptide of claim 17 wherein the N-terminus is the N-terminus of HAHB4.

19. A polypeptide of claim 18 wherein said polypeptide is capable of conferring stress tolerance in a plant.

20. A method for conferring stress tolerance in a plant which comprises introducing and expressing in a plant a nucleic acid construct as defined in claim 11.

21. A method for identifying a nucleic acid sequence which confers stress tolerance when introduced into a plant, which comprises using a nucleic acid sequence comprising a sequence of SEQ. ID. NO: 1, 2, 6, 7, or a part thereof or a sequence encoding for a sequence having homology to the sequence of SEQ. ID. NO. 11, 12 or 14, or a nucleic acid encoding all or a selected part of SEQ. ID. NO: 8 or 13 or a nucleic acid encoding all or a selected part of SEQ. ID. NO: 5, to probe a plant genome or plant genomic clones in a library.

22. An isolated nucleic acid sequence obtained or obtainable by the method of claim 21.

23. A nucleic acid sequence homologuous to the HAHB1 sequence as defined in SEQ ID NO. 6 or 7 wherein said gene sequence is capable of conferring stress tolerance when introduced and expressed in a plant.

24. A nucleic acid sequence as defined in claim 23 wherein said sequence shows at least 80%, preferably at least 90%, more preferably at least 95% homology to the HAHB1 sequence comprising SEQ ID NO. 6 or 7.

25. A nucleic acid sequence as defined in claim 24 wherein said sequence encodes for a sequence comprising a sequence with homology to the N-terminal homeodomain consensus motif as defined in SEQ ID NO. 14.

26. A nucleic acid sequence as defined in claim 25 wherein said sequence encodes for a sequence comprising a sequence with homology to the C-terminal consensus motif as defined in SEQ ID NO. 11 and/or the C-terminal consensus motif as defined in SEQ ID NO. 12.

27. A nucleic acid sequence as defined in claim 26 wherein said homology is at least 80%, preferably at least 90%, more preferably at least 95%.

28. A method for producing a stress tolerant plant or enhancing stress tolerance of a plant comprising transforming a plant with a nucleic acid sequence of claim 27.

29. A method for producing a stress tolerant plant or enhancing stress tolerance of a plant comprising transforming a plant with a nucleic acid sequence encoding for PR2.

30. A method for producing a stress tolerant plant or enhancing stress tolerance of a plant comprising transforming a plant with a nucleic acid sequence encoding for gluc.

31. A method for producing a stress tolerant plant or enhancing stress tolerance of a plant comprising transforming a plant with a nucleic acid sequence encoding for PR4.

32. The method of claim 29 wherein said tolerance is freezing tolerance.

33. An isolated nucleic acid sequence comprising a nucleic acid sequence of SEQ ID. No. 1, a functional fragment or a functional variant thereof.

34. An isolated nucleic acid sequence comprising a nucleic acid sequence of SEQ ID. No. 7.

35. An isolated nucleic acid sequence comprising a nucleic acid sequence of SEQ ID. No. 2.

36. An isolated polypeptide sequence comprising a sequence of SEQ ID. No. 5.

37. A vector comprising a gene construct comprising a sequence of claim 34, a functional part or functional variant thereof.

38. A vector comprising a gene construct comprising a nucleic acid sequence of SEQ ID No. 6, a functional part or functional variant thereof.

39. A vector comprising a gene construct comprising a sequence expressing a protein of SEQ ID NO. 5, a functional part or functional variant thereof.

40. A vector according to claim 39 wherein said sequence is operably linked to a promoter sequence.

41. A vector according to claim 40 wherein said promoter regulates constitutive expression of the gene.

42. A vector according to claim 41 wherein the promoter is the 35S promoter.

43. A vector according to claim 42 wherein said promoter is the native HAHB1 promoter.

44. A vector according to claim 43 wherein said promoter comprises a nucleic acid sequence of SEQ ID. No. 1, a functional fragment or a functional variant thereof.

45. A host cell transformed with a vector as defined in claim 37.

46. A host cell expressing a protein of SEQ ID NO. 5, a functional part or functional variant thereof.

47. A host cell according to claim 46 wherein the host cell is a plant cell.

48. A transgenic plant transformed with a vector as defined in claim 37.

49. A method for conferring stress tolerance in a plant comprising introducing and expressing in a plant a nucleic acid sequence of SEQ ID No. 2, 6 or 7, a functional part, or functional variant thereof.

50. A method according to claim 49 wherein said stress tolerance is selected from freezing tolerance, enhanced low-temperature tolerance, enhanced chilling tolerance, enhanced tolerance to drought and/or enhanced tolerance to conditions of high salinity.

51. A method for inducing the production of antifreeze proteins (AFPs) in a plant comprising transforming a plant with a nucleic acid sequence of SEQ ID No. 2, 6 or 7, a functional part, or functional variant thereof.

52. A method according to claim 51 wherein the antifreeze protein is selected form PR2, PR4 or glucanase.

53. (canceled)

54. A method for conferring stress induced gene expression in a plant wherein said method comprises transforming a plant with an expression cassette comprising a nucleic acid sequence of SEQ ID No. 1, a functional fragment or a functional variant thereof, operably linked to a gene sequence for expression.


SEQUENCE LISTING (81 sequences)

SEQ ID NO: 1 - DNA, 1011 bp, Helianthus annuus
gtcgagctcgtctcgtaaaatgttcgagtcagctccaattaaatcatgtcggcttgttatattttttttaatttatttttgatattttttacatatatttataacataaaaaacaaaataaaaataaaaattacacatatatctatatgtattatttttctaaaattttaaatagcgaaagacatattaaaagtattatatgtataattttgtttagcttcccatatttttatatgttattaattaattaaacttaaaatgttaacactttaacacctcttacatactttttagttcaatacatttaaaattaaaattaatctatatgcaataaaataattcaagcaggcttgcaagctcacgagtcgagccatgcctggctcgagctcgactcatttacaaatcgagccaccaagccgactcgtttataaccgagttttttttagccgagtttttttcaagcgaacttcaaacaagtcacgcgcttttaattaacacattctagtctaaagaagataattgaaagagaaagtagatataagtaaaaggagtagccaaagatataaatttagggtctaaacaacctaatattgtttaattttttttaaataaactagtttttttttaccgattatctgtgttatatgtcttagtttgacatgataagttatcataattacttgtagtatttttatatcagaaatatacgttggagaattaaattttatcctgatcgtcaattgacaagaacaaaaatcaacatctcatggttttttactaatttatatgattaaagatatatggttgtaagaaaaagaacaatgtacatcaaatggtgaaatttgaatatttgatagtaacgtaatccattgtgtatttcttattattttatcattttcccaaggtgtgtcatatatagtgtctccattctttctatagcacaatatccttcacctccctctctctctctctctctctaaaaatgatgatgagacgacaaagatcgaattc

SEQ ID NO: 2 - RNA, 1195 nt, Helianthus annuus
uugaaauucugagaaaagccacauaaucaaagcuaaagaggugguuuaaacagcugaugacuugcacuggaauggcuuucuucuccuccaauuucauguuacaauccucccaagaagaugaccaucaugccccuacaucucucucuccaauccucccaccuugcaguaccaccacucaagauuucaguggugcugcuuucuugggaaaaagaucuaugucuucuuacucagguuugaacaacaacaacauggauggaugugaucaagaagggaacaugaauggagaagaugaguuaucagaugauggaucacagcuucuugcaggagagaaaaagaggagauuaaacauggaacaagugaagacacuugagagaaacuuugaguuaggaaauaagcuugaaccugagaggaaaaugcaacuugcaagagcacuuggacuacaaccaagacagauugcuauaugguuucaaaacagaagagcuagauggaaaacuaaacaguuggaaaaagacuaugaugcccucaagagacaguuugaagcuguuaaagcugagaaugauucacuccaaucucaaaaucauaaacuucaugcugagauaauggcacuaaaaaauagggagccagcagaacuaaucaaccucaacauaaaagaaacagaaggaucuugcagcaaccgaagcgaaaacagcucugaaaucaaacuagacaucucaagaacaccggcuaccgauagcccuuuaucaucacaccaucaacaccaacaccagccaauaccuaaucuuuuuccaucgucgaauaucgauaggccuaauucgaauaacauuguggcgcaucaacuuuuccacaauucgucaucaaggccggcagaucaucaacuucauugccacaaacucgaucaaucgaaugccauuaaagaagaauguuuuagcacaauguuuguugguauggaugaucaaucaggguuuuggccaugguuggaacaaccacaauucaauugauggaaucaagaagcaaaaaagcaaaagaaaacgguacccgauucgccuucuuggcuuugguuugauuauauuaaagauggagaucaucaaucuguuuguucucuaagcuuuaaauucuuguuuuuugguacuuaaauuaauagaguaaaaauuagaagaaaaaacguauuauuauuuuaaauucaagauuaguguuu

SEQ ID NO: 3 - DNA, 315 bp, Helianthus annuus
aattaactcaccttaactaagttacttatgacaacatctctctcatagatcttgatgcagcttgcattcatgagttgtgatgtacaactcattcatgcattagggtttcagttttttcaaagttttttttttattttttcttctgtttcaagatcatgatgatgagttgtgctgaacacttgaacagctcattgatgcattagggtttgttttagtttcaagttctttcttttctttcattttcatgcactaaatccatatgggcttgaagaaagtttgaatctttatatgttagttgatgatcttgatgcaggt

SEQ ID NO: 4 - DNA, 778 bp, Helianthus annuus
gtaatattagtttgattgtttattgcatctatcaatcattagattctactctttacttgatcacacagaaagtaactaaaccttttttcctaatgataacaatatttgttttgcaaatctaatggcaatcaaataaaagtttctggtaagcagccatgatctatttatttttcactatttgagtaagtttaaaagttgcatttatcctcactaattatatacaacactaaaataatcattaaactgactgttataattactttccgtaaacggtatgccaaaacttaaaatgattaacaattttataagaatggaaagtaaaatcattacactatttcccatattagtcatgaccaaagtttgtttctttctgaagggcaaaagggtcaatatgcttatatgcagcatgggcaaaagaagtagagtgtatatcaaaattcatatctttattttcttttcaaagtttaggtaacaaaaagaagaaattataaacgagtttgttacaattccacaagtacatgaagaaacaaaatttgttagtatttttattttccatgtttttagtaacttccatatcaatttagcactagaagataactttttttaggactcggtaaaccatacaagtagggtcatactttatcgtttatccattaatgtatatccataaattcactgattatgcggtatttcctttgttacactgtcttgaacaagtattagtacatgtagtttcttaaagattgtttaatcaaccaaaaagattgaaactttgcag

SEQ ID NO: 5 - protein, 313 aa, Helianthus annuus
MTCTGMAFFSSNFMLQSSQEDDHHAPTSLSPILPPCSTTTQDFSGAAFLGKRSMSSYSGLNNNNMDGCDQEGNMNGEDELSDDGSQLLAGEKKRRLNMEQVKTLERNFELGNKLEPERKMQLARALGLQPRQIAIWFQNRRARWKTKQLEKDYDALKRQFEAVKAENDSLQSQNHKLHAEIMALKNREPAELINLNIKETEGSCSNRSENSSEIKLDISRTPATDSPLSSHHQHQHQPIPNLFPSSNIDRPNSNNIVAHQLFHNSSSRPADHQLHCHKLDQSNAIKEECFSTMFVGMDDQSGFWPWLEQPQFN

SEQ ID NO: 6 - DNA, 1195 bp, Helianthus annuus
ttgaaattctgagaaaagccacataatcaaagctaaagaggtggtttaaacagctgatgacttgcactggaatggctttcttctcctccaatttcatgttacaatcctcccaagaagatgaccatcatgcccctacatctctctctccaatcctcccaccttgcagtaccaccactcaagatttcagtggtgctgctttcttgggaaaaagatctatgtcttcttactcaggtttgaacaacaacaacatggatggatgtgatcaagaagggaacatgaatggagaagatgagttatcagatgatggatcacagcttcttgcaggagagaaaaagaggagattaaacatggaacaagtgaagacacttgagagaaactttgagttaggaaataagcttgaacctgagaggaaaatgcaacttgcaagagcacttggactacaaccaagacagattgctatatggtttcaaaacagaagagctagatggaaaactaaacagttggaaaaagactatgatgccctcaagagacagtttgaagctgttaaagctgagaatgattcactccaatctcaaaatcataaacttcatgctgagataatggcactaaaaaatagggagccagcagaactaatcaacctcaacataaaagaaacagaaggatcttgcagcaaccgaagcgaaaacagctctgaaatcaaactagacatctcaagaacaccggctaccgatagccctttatcatcacaccatcaacaccaacaccagccaatacctaatctttttccatcgtcgaatatcgataggcctaattcgaataacattgtggcgcatcaacttttccacaattcgtcatcaaggccggcagatcatcaacttcattgccacaaactcgatcaatcgaatgccattaaagaagaatgttttagcacaatgtttgttggtatggatgatcaatcagggttttggccatggttggaacaaccacaattcaattgatggaatcaagaagcaaaaaagcaaaagaaaacggtacccgattcgccttcttggctttggtttgattatattaaagatggagatcatcaatctgtttgttctctaagctttaaattcttgttttttggtacttaaattaatagagtaaaaattagaagaaaaaacgtattattattttaaattcaagattagtgttt

SEQ ID NO: 7 - DNA, 3299 bp, Helianthus annuus
gtcgagctcgtctcgtaaaatgttcgagtcagctccaattaaatcatgtcggcttgttatattttttttaatttatttttgatattttttacatatatttataacataaaaaacaaaataaaaataaaaattacacatatatctatatgtattatttttctaaaattttaaatagcgaaagacatattaaaagtattatatgtataattttgtttagcttcccatatttttatatgttattaattaattaaacttaaaatgttaacactttaacacctcttacatactttttagttcaatacatttaaaattaaaattaatctatatgcaataaaataattcaagcaggcttgcaagctcacgagtcgagccatgcctggctcgagctcgactcatttacaaatcgagccaccaagccgactcgtttataaccgagttttttttagccgagtttttttcaagcgaacttcaaacaagtcacgcgcttttaattaacacattctagtctaaagaagataattgaaagagaaagtagatataagtaaaaggagtagccaaagatataaatttagggtctaaacaacctaatattgtttaattttttttaaataaactagtttttttttaccgattatctgtgttatatgtcttagtttgacatgataagttatcataattacttgtagtatttttatatcagaaatatacgttggagaattaaattttatcctgatcgtcaattgacaagaacaaaaatcaacatctcatggttttttactaatttatatgattaaagatatatggttgtaagaaaaagaacaatgtacatcaaatggtgaaatttgaatatttgatagtaacgtaatccattgtgtatttcttattattttatcattttcccaaggtgtgtcatatatagtgtctccattctttctatagcacaatatccttcacctccctctctctctctctctctctaaaaatgatgatgagacgacaaagatcgaattcttgaaattctgagaaaagccacataatcaaagctaaagaggtggtttaaacagctgatgacttgcactggaatggctttcttctcctccaatttcatgttacaatcctcccaagaagatgaccatcatgcccctacatctctctctccaatcctcccaccttgcagtaccaccactcaagatttcagtggtaattaactcaccttaactaagttacttatgacaacatctctctcatagatcttgatgcagcttgcattcatgagttgtgatgtacaactcattcatgcattagggtttcagttttttcaaagttttttttttattttttcttctgtttcaagatcatgatgatgagttgtgctgaacacttgaacagctcattgatgcattagggtttgttttagtttcaagttctttcttttctttcattttcatgcactaaatccatatgggcttgaagaaagtttgaatctttatatgttagttgatgatcttgatgcaggtgctgctttcttgggaaaaagatctatgtcttcttactcaggtttgaacaacaacaacatggatggatgtgatcaagaagggaacatgaatggagaagatgagttatcagatgatggatcacagcttcttgcaggagagaaaaagaggagattaaacatggaacaagtgaagacacttgagagaaactttgagttaggaaataagcttgaacctgagaggaaaatgcaacttgcaagagcacttggactacaaccaagacagattgctatatggtttcaaaacagaagagctagatggaaaactaaacagttggaaaaagactatgatgccctcaagagacagtttgaagctgttaaagctgagaatgattcactccaatctcaaaatcataaacttcatgctggtaatattagtttgattgtttattgcatctatcaatcattagattctactctttacttgatcacacagaaagtaactaaaccttttttcctaatgataacaatatttgttttgcaaatctaatggcaatcaaataaaagtttctggtaagcagccatgatctatttatttttcactatttgagtaagtttaaaagttgcatttatcctcactaattatatacaacactaaaataatcattaaactgactgttataattactttccgtaaacggtatgccaaaacttaaaatgattaacaattttataagaatggaaagtaaaatcattacactatttcccatattagtcatgaccaaagtttgtttctttctgaagggcaaaagggtcaatatgcttatatgcagcatgggcaaaagaagtagagtgtatatcaaaattcatatctttattttcttttcaaagtttaggtaacaaaaagaagaaattataaacgagtttgttacaattccacaagtacatgaagaaacaaaatttgttagtatttttattttccatgtttttagtaacttccatatcaatttagcactagaagataactttttttaggactcggtaaaccatacaagtagggtcatactttatcgtttatccattaatgtatatccataaattcactgattatgcggtatttcctttgttacactgtcttgaacaagtattagtacatgtagtttcttaaagattgtttaatcaaccaaaaagattgaaactttgcagagataatggcactaaaaaatagggagccagcagaactaatcaacctcaacataaaagaaacagaaggatcttgcagcaaccgaagcgaaaacagctctgaaatcaaactagacatctcaagaacaccggctaccgatagccctttatcatcacaccatcaacaccaacaccagccaatacctaatctttttccatcgtcgaatatcgataggcctaattcgaataacattgtggcgcatcaacttttccacaattcgtcatcaaggccggcagatcatcaacttcattgccacaaactcgatcaatcgaatgccattaaagaagaatgttttagcacaatgtttgttggtatggatgatcaatcagggttttggccatggttggaacaaccacaattcaattgatggaatcaagaagcaaaaaagcaaaagaaaacggtacccgattcgccttcttggctttggtttgattatattaaagatggagatcatcaatctgtttgttctctaagctttaaattcttgttttttggtacttaaattaatagagtaaaaattagaagaaaaaacgtattattattttaaattcaagattagtgttt

SEQ ID NO: 8 - protein, 122 aa, Helianthus annuus
LINLNIKETEGSCSNRSENSSEIKLDISRTPATDSPLSSHHQHQHQPIPNLFPSSNIDRPNSNNIVAHQLFHNSSSRPADHQLHCHKLDQSNAIKEECFSTMFVGMDDQSGFWPWLEQPQFN

SEQ ID NO: 9 - protein, 35 aa, Helianthus annuus
LINLNIKETEGSCSNRSENSSEIKLDISRTPATDS

SEQ ID NO: 10 - protein, 29 aa, Helianthus annuus
IKEECFSTMFVGMDDQSGFWPWLEQPQFN

SEQ ID NO: 11 - protein, 34 aa, Helianthus annuus
SINLNKETEGSCSNRSENSSDIKLDISRTPAIDS

SEQ ID NO: 12 - protein, 29 aa, Helianthus annuus
VKEESLSNMFCGIDDQSGFWPWLEQQHFN

SEQ ID NO: 13 - protein, 91 aa, Helianthus annuus
MTCTGMAFFSSNFMLQSSQEDDHHAPTSLSPILPPCSTTTQDFSGAAFLGKRSMSSYSGLNNNNMDGCDQEGNMNGEDELSDDGSQLLAGE

SEQ ID NO: 14 - protein, 57 aa, Helianthus annuus
KKRRLTDEQVKALEKSFELENKLEPERKVQLARELGLQPRQVAVWFQNRRARWKTKQ

SEQ ID NO: 15 - DNA, 33 bp, Artificial
  Primer PrH1R
cggggatcccctctttagctttgattatgtggc

SEQ ID NO: 16 - DNA, 16 bp, Artificial
  Primer PR-TOPO
aacagctatgaccatg

SEQ ID NO: 17 - DNA, 21 bp, Artificial
  Primer H1qF
ggccggcagatcatcaacttc

SEQ ID NO: 18 - DNA, 21 bp, Artificial
  Primer H1qR
ccaaccatggccaaaaccctg

SEQ ID NO: 19 - DNA, 32 bp, Artificial
  Primer PR4 CDS-F
ggcggatccccaccaagaaaacaaagacttat

SEQ ID NO: 20 - DNA, 27 bp, Artificial
  Primer PR4 CDS-R
ggggagctcccgatcgatattgacctc

SEQ ID NO: 21 - DNA, 30 bp, Artificial
  Primer PR2 CDS-F
ggcggatccaagaaaatgtctgaatcaagg

SEQ ID NO: 22 - DNA, 27 bp, Artificial
  Primer PR2 CDS-R
ggggagctcgcccacaagtctctaagg

SEQ ID NO: 23 - DNA, 31 bp, Artificial
  Primer Glucanase CDS-F
cgcggatccctaaggagctaagaacaaaccc

SEQ ID NO: 24 - DNA, 29 bp, Artificial
  Primer Glucanase CDS-R
cccgagctcatcactcaaccgccgtaccg

SEQ ID NO: 25 - DNA, 24 bp, Artificial
  Primer tc23839-F
aaaagtggtttatttggatgagga

SEQ ID NO: 26 - DNA, 22 bp, Artificial
  Primer tc23839-R
gacacgtcagaacaaaattcca

SEQ ID NO: 27 - DNA, 25 bp, Artificial
  Primer tc19654-F
gatcttcgtgacctgcttctaaaac

SEQ ID NO: 28 - DNA, 26 bp, Artificial
  Primer tc19654-R
caaaccacttctaaatcatcccatag

SEQ ID NO: 29 - DNA, 26 bp, Artificial
  Primer tc16546-F
caaaacaacttcttccaccaatagtc

SEQ ID NO: 30 - DNA, 25 bp, Artificial
  Primer tc16546-R
aataaaccgttgacttttcttcacc

SEQ ID NO: 31 - DNA, 24 bp, Artificial
  Primer tc18434-F
acatcatcaacggtggtttagaat

SEQ ID NO: 32 - DNA, 25 bp, Artificial
  Primer 18434-R
acatggtgcaataccttctgtaaaa

SEQ ID NO: 33 - DNA, 24 bp, Artificial
  Primer At3g12500-F
gggttatggagtgattacgaacat

SEQ ID NO: 34 - DNA, 24 bp, Artificial
  Primer At3g12500-R
taccaccaggattaacaccaaata

SEQ ID NO: 35 - DNA, 24 bp, Artificial
  Primer At3g57260-R
taagaaggaaccaacgtatgagaa

SEQ ID NO: 36 - DNA, 24 bp, Artificial
  Primer At3g57260-R
cataaaaagcccacaagtctctaa

SEQ ID NO: 37 - DNA, 24 bp, Artificial
  Primer At3g04720-F
attgaacattgctacatccaaatc

SEQ ID NO: 38 - DNA, 24 bp, Artificial
  Primer At3g04720-R
attgaacattgctacatccaaatc

SEQ ID NO: 39 - DNA, 20 bp, Artificial
  Primer At1g62440-F
accaacaccaccttctctgc

SEQ ID NO: 40 - DNA, 20 bp, Artificial
  Primer At1g62440-R
ttgatggtggaggaggagac

SEQ ID NO: 41 - DNA, 22 bp, Artificial
  Primer At2g43050-F
ggttaaatggagtgggtgtcat

SEQ ID NO: 42 - DNA, 22 bp, Artificial
  Primer At2g43050-R
caagtcctgggtcgaaactaac

SEQ ID NO: 43 - DNA, 24 bp, Artificial
  Primer At4g16260-R
gagacctggaagaggagtggaaac

SEQ ID NO: 44 - DNA, 24 bp, Artificial
  Primer At4g16260-R
aatgtgatcggaaattttggttgt

SEQ ID NO: 45 - DNA, 24 bp, Artificial
  Primer At4g25480-F
gacgttggtggaggctatttacac

SEQ ID NO: 46 - DNA, 24 bp, Artificial
  Primer At4g25480-R
tattagccaacaaactcggcatct

SEQ ID NO: 47 - DNA, 24 bp, Artificial
  Primer At4g02380-F
atgctatcttccgacgtggttatg

SEQ ID NO: 48 - DNA, 24 bp, Artificial
  Primer At4g02380-R
cttccactcccttcttcttcatca

SEQ ID NO: 49 - DNA, 22 bp, Artificial
  Primer At4g25490-R
cgttggcttttcaagatgagac

SEQ ID NO: 50 - DNA, 24 bp, Artificial
  Primer At4g25490-R
cgctctgttccggtgtataaatag

SEQ ID NO: 51 - DNA, 23 bp, Artificial
  Primer At1g27730-F
gtccactagccacgttagcagta

SEQ ID NO: 52 - DNA, 23 bp, Artificial
  Primer At1g27730-R
agttgaagtttgaccggaaagtc

SEQ ID NO: 53 - DNA, 24 bp, Artificial
  Primer At3g04720-F
attgaacattgctacatccaaatc

SEQ ID NO: 54 - DNA, 24 bp, Artificial
  Primer At3g12500-F
gggttatggagtgattacgaacat

SEQ ID NO: 55 - DNA, 24 bp, Artificial
  Primer At3g12500-R
taccaccaggattaacaccaaata

SEQ ID NO: 56 - DNA, 25 bp, Artificial
  Primer At3g52130-F
tttctctttaataaccttgctgctt

SEQ ID NO: 57 - DNA, 24 bp, Artificial
  Primer At3g52130-R
gctaatgactgagattttgattcg

SEQ ID NO: 58 - DNA, 24 bp, Artificial
  Primer At3g07450-F
gacacttggtcaaccttgtttatg

SEQ ID NO: 59 - DNA, 24 bp, Artificial
  Primer At3g07450-R
tacatggaagaaaattggcagaac

SEQ ID NO: 60 - DNA, 25 bp, Artificial
  Primer At5g44420-F
cttgttctctttgctgctttcgacg

SEQ ID NO: 61 - DNA, 24 bp, Artificial
  Primer At5g44420-R
cttcaaggttaatgcactgattct

SEQ ID NO: 62 - DNA, 24 bp, Artificial
  Primer At3g57260-F
taagaaggaaccaacgtatgagaa

SEQ ID NO: 63 - DNA, 24 bp, Artificial
  Primer At3g57260-R
cataaaaagcccacaagtctctaa

SEQ ID NO: 64 - DNA, 1813 bp, Arabidopsis thaliana
accagaagtggtatagtctaggccgatacatttcactatctctctctcttttgtttttcctcttcttctttttttccatttgatttcaaactctcacacaaagagcttcagatttataagaccatgataatggctttaagacaaagattggcaagaagaaaaaactaaagagaaacgaccaaaatctcaagcaaacagtactaacttctgttgcaaaacagaagaagatgtcttgtaataatggaatgtcttttttcccttcaaatttcatgatccaaacctcttacgaagatgatcatcctcatcaatctccatctcttgctcctcttcttccttcttgctctctacctcaagatctccatggtatatatacataaacttccacacacatctcctctgttttctctctatctctttctaatgctctgttctgttctgtttcaggatttgcttcgtttctaggtaagagatctccaatggaagggtgttgtgatttagaaacagggaacaatatgaatggagaagaggattattcagatgatgggtcacaaatgggagagaagaagaggagattgaacatggaacaagtgaagacactagagaagaactttgagcttggaaacaaacttgaaccagagaggaaaatgcagctagctcgtgccttaggtttgcaaccaagacagatcgcgatttggtttcaaaatcgaagagctcgttggaaaacaaagcagctagagaaagattatgatactcttaaacgacagtttgatacacttaaagctgaaaatgatcttcttcaaactcataatcagaaactccaagctgaggtaattaatctcataaattaacaaaaaaaatcaatatgtgttatttttttttgggttaatgatcaataattacagttattttccatctaaaggatgatttttttctttttaaaaaaggttaaaaattatatttctggtttataattatttggatcaggagttgctttcaggtagggttaaaaaactggacatgattcatgacttttcagacatcattatctctttttttcttcactcttgtctggaaagagatctgaaaacaatagtttctttatgcttatcacattgtacagtaactctgtttatgtttaaaattttgtctttaattacgcagataatgggattaaaaaacagagaacaaacagaatcaataaatctaaacaaagaaactgaaggatcttgcagtaacagaagtgataacagttcagataatctcagactagatatctcaactgcgccgccatcaaacgacagtacattaaccggtggccacccaccgccaccacagacagttggtcgacacttcttcccaccgtcgccagccaccgcaacgacaactactacaacaatgcagttctttcaaaactcatcttcaggacagagtatggttaaagaagagaatagtatcagtaacatgttctgtgcaatggatgaccattctggtttttggccatggcttgatcagcaacagtacaattgaaattggtctacctgttttttttgtttttgtttttaaaaaaatttatattttttttttttgtatttggaattttgatcagaagaacccatgcatgttttcaaaaactggaatctatatcattagctcactttgaaatctgcaaccaaacaccactgaggttttttgtttactttttgagtaaatgagatgtaaaaaaatgggtaatatccattatattatataaaaaataatatcattatggcccaacatttttctgtatggagaaaaataaaataaattgtatatt

SEQ ID NO: 65 - DNA, 1544 bp, Arabidopsis thaliana
ctactaaaaaaattgtaagtacatacatatcaatgttaatttgtatataaggagctaagaacaaacccaattaggcaactagcaattgctaaaacacgtaagatctcaaatatgaccacgttattcctccttattgctctattcatcacaaccatcctcaacccaacaagtctctctctctctctctttaatctcacatgatttattctcattttcaatttttatagatatataaacttaatcataattaaccttaattatgttatatgataaactaggtggagaatcagtaggtgtatgctatggaatgatggggaacaaccttccttctcaatcagacacaatcgctctctttagacaaaacaacatccgacgtgttagactctacgatccaaaccaagccgctttaaacgctcttagaaacacgggtatcgaagtcatcatcggcgttccaaacaccgatcttcgttcactcactaacccttcttccgctagatcatggctccaaaacaacgtcctcaactattaccccgccgttagcttcaagtacatcgccgtaggtaacgaagtatctccgtcgaacggcggtgatgttgtgctccctgccatgcgtaacgtttacgatgctctaagaggtgcaaatcttcaagatcgtattaaagtttctaccgccattgatatgactttgattggaaactctttccctccttcctccggagagtttcgtggtgacgttagatggtatatcgatcccgtcatcgggtaattttccaaccaaaccaaataaccaaaattcattagatttagttatttcccaatatttttcatttctggttacttgtggaatgattatttaatttcttcctatgtggctaattaggtttcttacgagtacgaactcagcgttactagccaacatctatccttacttcagctacgttgacaatccacgtgacatatctctctcttacgctctcttcacttctccttccgtcgtcgtatgggacggctctcgtggctaccaaaacctctttgacgctttacttgacgttgtttactctgccgttgaacgctcaggcggtggatctctcccagtggttgtttccgagagcggatggccttctaacggtggaaacgccgcgagtttcgataacgcgcgaagctttttacacgaatcttgcgtcgcgtgtgagagagaacagaggaacaccgaagagacctggaagaggagtggaaacgtatttgttcgctatgtttgatgagaatcaaaagagtcctgagatcgagaagaattttggtttgttttttcctaataaacaaccaaaatttccgatcacattctctgccgcgagagacggtacggcggttgagtgatgattttatatgctgagatttatgtgaataattgggagattatcccataaaaggttccaaataaagacaaatttcaaataaaacctgttagtccaagttaaattaaatactcggctttgttttggtccacgttagacttggtaaagtcatgcaatatttttatttgatat

SEQ ID NO: 66 - DNA, 1343 bp, Arabidopsis thaliana
atatcatttttcacagaatcatagaaaaatcaagaaaatgtctgaatcaaggagcttagcctcaccaccaatgttgatgattcttctcagccttgtaatagcttccttcttcaaccacacaggttcagtcatcttttaagctattgtaacatctattaatcatctccatcttcacaaatttattcaatttaatgattcttattttggaaaatgaagctggacaaatcggagtatgctacgggatgctaggcgataccttgccaagtccatcggacgttgtggctctttacaaacaacaaaacatccagcgaatgcggctctacggccctgacccaggcgctcttgccgctctccgtggctctgacatcgagctcatcctcgacgttcccagttcagatcttgaacgtctcgcctccagtcaaacggaggccgacaagtgggttcaagaaaacgttcagagctacagagatggtgtcagattccggtacatcaacgttggaaatgaggtgaaaccctcagttggggggtttctcttacaagcaatgcagaacatcgagaacgcggtttctggagcagggcttgaagtcaaggtctcaacagctatagccactgacaccaccactgatacgtctcctccgtctcaaggaaggttcagggatgagtataagagctttctcgaaccagtgataggtttcttggcaagcaagcaatctcccttgctcgtgaatctctacccttacttcagctacatgggagacacggccaacatccatctagactacgctctgttcaccgcccagtccactgttgataacgatccagggtactcataccaaaacctattcgacgcaaatctcgactcggtttatgcagcattggagaaatcagggggcggatcgttggaaatcgtggtgtcggagaccggttggcccacagagggagcagtcgggacgagtgtggaaaacgcaaagacttatgttaacaatttgatacaacatgtgaagaatggatcaccgagaaggccagggaaagctatagagacttatatattcgctatgttcgatgagaataagaaggaaccaacgtatgagaagttttggggactgtttcatccagatcgacagtctaagtatgaagttaatttcaactaatccttagagacttgtgggctttttatgtaagcgtatttaaaaattgggaacttgttgtagtaataaggaataattaatgcgctttcagcgtgtagtatgttgttatttttaaggttataaatgagctgcaagcataaataaggaaaaaaaatagcatgggcctataggcccaataataaaacaagcttgctt

SEQ ID NO: 67 - DNA, 998 bp, Arabidopsis thaliana
agaccaccaagaaaacaaagacttatcgatcatgaagatcagacttagcataaccatcatacttttatcatacacagtggctacggtggccggacaacaatgcggtcgtcaaggcggtggtcgaacttgtcccggtaacatctgctgcagtcagtacggttactgtggtaccaccgcggactactgttctccgaccaacaactgtcagagcaattgttggggaagtgggcctagcggaccaggggagagcgcgtcgaacgtacgcgccacctaccatttctataatccggcgcagaataattgggatttgagagccgtgagtgcttattgctccacgtgggatgctgataagccgtacgcatggcggagcaagtatggctggaccgccttctgcgggccggcaggacctcgtggtcaagcttcttgcggcaagtgtttaagggtaagttaattaattatctttttctcaaatctttatataagtatgtttgtgcaaaaggagatcatatagaaagtgttggaattaagacgaatacaagataaaatttgttaccatttaccaacgtcaacgtgttagtgaaatatttcaaaagatgtatagccggtaaaaattgtgattaaccggtgggtataaatggattcaggtgaagaacacaagaacaaatgctgcagtaactgtgagaatagtggaccaatgcagcaacggaggcttggatttggatgtagcaatgttcaatcaaatagacaccgatggttttggctatcaacaaggccatctcattgttgactaccaatttgtcgactgtggcaatgagctcattgggcagcctgattccagaaacatgcttgtttcggccattgatcgcgtttgatattatgtaatgattttgaggtcaatatcgatcggtctacataaaaataataaagaccgctatatatgtattgtcgagggatatatgtttcgtatcaataaggaaattttaaatattattatcatt

SEQ ID NO: 68 - DNA, 20 bp, Artificial
  GmPR2qF
ccttcttctggtggaactgc

SEQ ID NO: 69 - DNA, 20 bp, Artificial
  GmPR2qR
ataggagaaaagagccccca

SEQ ID NO: 70 - DNA, 22 bp, Artificial
  NtPR2qF
ctggtttgggaaacaacatcaa

SEQ ID NO: 71 - DNA, 22 bp, Artificial
  NtPR2qR
aatctggcctggattaccagaa

SEQ ID NO: 72 - DNA, 22 bp, Artificial
  GmPR4qF
acaggaacaggagcaaacacaa

SEQ ID NO: 73 - DNA, 22 bp, Artificial
  GmPR4qR
cattcccacaatccacaaactg

SEQ ID NO: 74 - DNA, 22 bp, Artificial
  NtPR4qF
tttggcatggaggaggaagtat

SEQ ID NO: 75 - DNA, 22 bp, Artificial
  NtPR4qR
tccacgattctcactgtggtct

SEQ ID NO: 76 - DNA, 22 bp, Artificial
  GmGlucqF
gagaaagtaggggcaccaaatg

SEQ ID NO: 77 - DNA, 22 bp, Artificial
  GmGlucqR
ttctggtttccatcaaacatgg

SEQ ID NO: 78 - DNA, 24 bp, Artificial
  GmEF1aqF
tgaaacagatgatttgctgctgta

SEQ ID NO: 79 - DNA, 24 bp, Artificial
  GmEF1aqR
caatcatgttgtctccctcaaaac

SEQ ID NO: 80 - DNA, 24 bp, Artificial
  NtActqF
ctgatggacaggttatcaccattg

SEQ ID NO: 81 - DNA, 24 bp, Artificial
  NtActqR
taatgcggtaatttccttgctcat